(19) United States
(12) Patent Application Publication
(10) Pub. No.: US 2016/0175306 A1
(43) Pub. Date: Jun. 23, 2016

(54) COMBINATION OF A IMIDAZOPYRIDAZINE DERIVATIVE AND A MITOTIC AGENT FOR THE TREATMENT OF CANCER

(71) Applicant: BAYER PHARMA AKTIENGESELLSCHAFT, Berlin (DE)

(72) Inventors: Antje Margret WENGNER, Berlin (DE); Gerhard SIEMEISTER, Berlin (DE)

(21) Appl. No.: 14/898,421

(22) PCT Filed: Jun. 11, 2014
(86) PCT No.: PCT/EP2014/062133

§ 371 Date: Dec. 14, 2015

(30) Foreign Application Priority Data
Jun. 13, 2013 (EP) 13171818.1

(51) Int. Cl. A61K 31/5025; A61K 31/475; A61K 31/337

(52) U.S. Cl. CPC A61K 31/5025; A61K 31/337; A61K 31/475

(57) ABSTRACT

The present invention relates to a combination comprising an Mps-1 kinase inhibitor and a mitotic inhibitor. The present invention also relates to the use of said combination for the treatment of cancer, in particular of pancreatic cancer, glioblastoma, ovarian cancer, non-small cell lung carcinoma, breast cancer and/or gastric cancer.

The present invention relates to a combination comprising an Mps-1 kinase inhibitor and a mitotic inhibitor. The present invention also relates to the use of said combination for the treatment of cancer, in particular of pancreatic cancer, glioblastoma, ovarian cancer, non-small cell lung carcinoma, breast cancer and/or gastric cancer.

### BACKGROUND OF THE INVENTION

Mps-1 (Monopolar Spindle 1) kinase (also known as Tyrosine Threonine Kinase, TTK) is a dual specificity Ser/Thr kinase which plays a key role in the activation of the mitotic checkpoint (also known as spindle checkpoint, spindle assembly checkpoint) thereby ensuring proper chromosome segregation during mitosis [Abrieu A et al., Cell, 2001, 106, 83-93]. Every dividing cell has to ensure equal separation of the replicated chromosomes into the two daughter cells. Upon entry into mitosis, chromosomes are attached at their kinetochores to the microtubules of the spindle apparatus. The mitotic checkpoint is a surveillance mechanism that is active as long as unattached kinetochores are present and prevents mitotic cells from entering anaphase and thereby completing cell division with unattached chromosomes [Suijkerbuijk S J and Kops G J, Biochemica et Biophysica Acta, 2008, 1786, 24-31; Musacchio A and Salmon E D, Nat Rev Mol Cell Biol., 2007, 8, 379-93]. Once all kinetochores are attached in a correct amphitelic, i.e. bipolar, fashion with the mitotic spindle, the checkpoint is satisfied and the cell enters anaphase and proceeds through mitosis. The mitotic checkpoint consists of a complex network of a number of essential proteins, including members of the MAD (mitotic arrest deficient, MAD 1-3) and Bub (Budding uninhibited by benzimidazole, Bub 1-3) families, the motor protein CENP-E, Mps-1 kinase as well as other components, many of these being over-expressed in proliferating cells (e.g. cancer cells) and tissues [Yuan B et al., Clinical Cancer Research, 2006, 12, 405-10]. The essential role of Mps-1 kinase activity in mitotic checkpoint signalling has been shown by shRNA-silencing, chemical genetics as well as chemical inhibitors of Mps-1 kinase [Jelluma N et al., PLos ONE, 2008, 3, e2415; Jones M H et al., Current Biology, 2005, 15, 160-65; Dorer R K et al., Current Biology, 2005, 15, 1070-76; Schmidt M et al., EMBO Reports, 2005, 6, 866-72].

There is ample evidence linking reduced but incomplete mitotic checkpoint function with aneuploidy and tumorigenesis [Weaver B A and Cleveland D W, Cancer Research, 2007, 67, 10103-5; King R W, Biochimica et Biophysica Acta, 2008, 1786, 4-14]. In contrast, complete inhibition of the mitotic checkpoint has been recognised to result in severe chromosome missegregation and induction of apoptosis in tumour cells [Kops G J et al., Nature Reviews Cancer, 2005, 5, 773-85; Schmidt M and Medema R H, Cell Cycle, 2006, 5, 159-63; Schmidt M and Bastians H, Drug Resistance Updates, 2007, 10, 162-81].

Based on these findings, Mps-1 has been considered as one among the most promising drug targets for cancer therapy.

Different compounds have been disclosed in prior art which show an inhibitory effect on Mps-1 kinase. WO2010/124826A1 discloses substituted imidazoquinoxaline compounds as inhibitors of Mps-1 kinase. WO2011/026579A1 discloses substituted aminoquinoxalines as Mps-1 kinase inhibitors. WO2011/063908A1, WO02011/064328A1 as well as WO2011/063907 A1 disclose triazolopyridine derivates as inhibitors of Mps-1 kinase.

WO 2011/013729A1 discloses fused imidazole derivatives as Mps-1 kinase inhibitors. Among the disclosed fused imidazole derivates there are also imidazo[1,2-b]pyridazines.

WO 2012/032031A1 inter alia is related to imidazo[1,2-b]pyridazines as Mps-1 kinase inhibitors.

However, the state of the art described above does not specifically describe the imidazopyridazine compounds as described and defined herein, and as hereinafter referred to as “compounds of the present invention”, or their pharmacological activity and stability.

It has been found, that said compounds of the present invention have surprising and advantageous properties. The compounds of the present invention surprisingly exhibit a superior overall profile with respect to Mps-1-related activity in a functional assay (Spindle Assembly Checkpoint Assay), antiproliferative activity (Proliferation Assay with HeLa cells), metabolic stability (in vitro metabolic stability in rat hepatocytes) and drug-drug interaction potential (inhibition of liver enzyme CYP3A4), as will be shown hereinafter.

Established anti-mitotic drugs such as vinca alkaloids, taxanes or epothilones activate the SAC either by stabilising or destabilising microtubule dynamics resulting in a mitotic arrest. This arrest prevents separation of sister chromatids to form the two daughter cells. Prolonged arrest in mitosis forces a cell either into mitotic exit without cytokinesis or into mitotic catastrophe leading to cell death. In contrast, inhibitors of Mps-1 induce a SAC inactivation that accelerates progression of cells through mitosis resulting in severe chromosomal missegregation and finally in cell death. Silencing of Mps-1 leads to failure of cells to arrest in mitosis in response to anti-mitotic drugs. Remarkably, combination of microtubule interfering agents and Mps-1 inhibition even increases chromosomal segregation errors and cell death (Abrieu A, Magnaghi-Jaulin L, Kahana J A, Peter M, Castro A, Vigneron S, Lorca T, Cleveland D W, Labbé J C. Mps1 is a kinetochore-associated kinase essential for the vertebrate mitotic checkpoint. Cell 2001; 106: 83-93, Stucke V M, Silljé H H, Arnaud L, Nigg E A. et at. Human Mps1 kinase is required for the spindle assembly checkpoint but not for centrosome duplication. EMBO J 2002; 21:1723-1732).

Therefore, the combined increase of chromosomal segregation errors induced by combination of anti-mitotics with SAC inhibition constitutes an efficient strategy for selectively eliminating tumor cells.

### SUMMARY OF THE INVENTION

The present invention covers a combination comprising: a compound A which is selected from:
N-cyclopropyl-4-{6-[1-(3-fluoro-4-methoxyphenyl)ethenyl]-8-[(3,3,3-trifluoropropyl)amino]imidazo[1,2-b]pyridazin-3-yl}-2-methylbenzamide,
N-cyclopropyl-4-{6-[difluoro(3-fluoro-4-methoxyphenyl)methyl]-8-[(3,3,3-trifluoropropyl)amino]imidazo[1,2-b]pyridazin-3-yl}-2-methylbenzamide,
(RS)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(R)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(S)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(RS)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(R)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(S)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(RS)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
(R)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
(S)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
N-cyclopropyl-4-{6-[difluoro(3-fluorophenyl)methyl]-8-[(3,3,3-trifluoropropyl)amino]imidazo[1,2-b]pyridazin-3-yl}-2-methylbenzamide,
N-cyclopropyl-4-{6-(3-methoxybenzoyl)-8-[(3,3,3-trifluoropropyl)amino]imidazo[1,2-b]pyridazin-3-yl}-2-methylbenzamide,
N-cyclopropyl-4-{6-[1-(3-methoxyphenyl)vinyl]-8-[(3,3,3-trifluoropropyl)amino]imidazo[1,2-b]pyridazin-3-yl}-2-methylbenzamide,
N-cyclopropyl-4-{6-(4-methoxybenzoyl)-8-[(3,3,3-trifluoropropyl)amino]imidazo[1,2-b]pyridazin-3-yl}-2-methylbenzamide,
N-cyclopropyl-4-{6-[1-(4-methoxyphenyl)vinyl]-8-[(3,3,3-trifluoropropyl)amino]imidazo[1,2-b]pyridazin-3-yl}-2-methylbenzamide,
N-cyclopropyl-4-{6-[(2, 5-difluorophenyl) (hydroxy)methyl]-8-[(3,3,3-trifluoropropyl)amino]imidazo[1,2-b]pyridazin-3-yl}-2-methylbenzamide,
N-cyclopropyl-4-{6-(2, 5-difluorobenzoyl)-8-[(3,3,3-trifluoropropyl)amino]imidazo[1,2-b]pyridazin-3-yl}-2-methylbenzamide,
N-cyclopropyl-4-{6-(3-fluoro-4-methoxybenzoyl)-8-[(3,3,3-trifluoropropyl)amino]imidazo[1,2-b]pyridazin-3-yl}-2-methylbenzamide,
N-cyclopropyl-4-{6-(2,3-difluorobenzoyl)-8-[(3,3,3-trifluoropropyl)amino]imidazo[1,2-b]pyridazin-3-yl}-2-methylbenzamide,
N-cyclopropyl-4-{6-[1-(2,3-difluorophenyl)vinyl]-8-[(3,3,3-trifluoropropyl)amino]imidazo[1,2-b]pyridazin-3-yl}-2-methylbenzamide,
N-cyclopropyl-4-{6-[difluoro(4-methoxyphenyl)methyl]-8-[(3,3,3-trifluoropropyl)amino]imidazo[1,2-b]pyridazin-3-yl}-2-methylbenzamide,
N-cyclopropyl-4-{6-[1-(2,3-difluorophenyl)cyclopropyl]-8-[(3,3,3-trifluoropropyl)amino]imidazo[1,2-b]pyridazin-3-yl}-2-methylbenzamide,
N-cyclopropyl-4-{6-[(2,3-difluorophenyl)(difluoro)methyl]-8-[(3,3,3-trifluoropropyl)amino]imidazo[1,2-b]pyridazin-3-yl}-2-methylbenzamide,
N-cyclopropyl-4-{6-[1-(2, 5-difluorophenyl)vinyl]-8-[(3,3,3-trifluoropropyl)amino]imidazo[1,2-b]pyridazin-3-yl}-2-methylbenzamide,
N-cyclopropyl-4-{6-[1-(2, 5-difluorophenyl)cyclopropyl]-8-[(3,3,3-trifluoropropyl)amino]imidazo[1,2-b]pyridazin-3-yl}-2-methylbenzamide,
N-cyclopropyl-4-{6-[(2, 5-difluorophenyl)(difluoro)methyl]-8-[(3,3,3-trifluoropropyl)amino]imidazo[1,2-b]pyridazin-3-yl}-2-methylbenzamide,
N-cyclopropyl-4-{6-[1-(5-fluoro-2-hydroxyphenyl)ethenyl]-8-[(3,3,3-trifluoropropyl)amino]imidazo[1,2-b]pyridazin-3-yl}-2-methylbenzamide,
N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide,
N-cyclopropyl-4-{6-(3-fluoro-4-methoxyphenoxy)-8-[(oxetan-3-ylmethyl)amino]imidazo[1,2-b]pyridazin-3-yl}-2-methylbenzamide,
N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide, and
N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide,
   or an N-oxide, a hydrate, a solvate, or a salt thereof, or a mixture of same;
   and
   one or more mitotic inhibitors.

The present invention further relates to the combination as defined supra, for use in the treatment or prophylaxis of cancer, in particular of pancreatic cancer, glioblastoma, ovarian cancer, non-small cell lung carcinoma, breast cancer and/or gastric cancer.

The present invention further relates to the use of the combination as defined supra, for the prophylaxis or treatment of cancer, in particular of pancreatic cancer, glioblastoma, ovarian cancer, non-small cell lung carcinoma, breast cancer and/or gastric cancer.

The present invention further relates to the use of the combination as defined supra, for the preparation of a medicament for the prophylaxis or treatment of cancer, in particular of pancreatic cancer, glioblastoma, ovarian cancer, non-small cell lung carcinoma, breast cancer and/or gastric cancer.

### DETAILED DESCRIPTION OF THE INVENTION

In accordance with a first aspect, the present invention relates to a combination comprising an Mps-1 kinase inhibitor, and one or more mitotic inhibitors.

The Mps-1 kinase inhibitor is selected from the group consisting of:
N-cyclopropyl-4-{6-[1-(3-fluoro-4-methoxyphenyl)ethenyl]-8-[(3,3,3-trifluoropropyl)amino]imidazo[1,2-b]pyridazin-3-yl}-2-methylbenzamide,
N-cyclopropyl-4-{6-[difluoro(3-fluoro-4-methoxyphenyl)methyl]-8-[(3,3,3-trifluoropropyl)amino]imidazo[1,2-b]pyridazin-3-yl}-2-methylbenzamide,
(RS)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(R)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(S)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(RS)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(R)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(S)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(RS)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
(R)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
(S)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
N-cyclopropyl-4-{6-[difluoro(3-fluorophenyl)methyl]-8-[(3,3,3-trifluoropropyl)amino]imidazo[1,2-b]pyridazin-3-yl}-2-methylbenzamide,
N-cyclopropyl-4-{6-(3-methoxybenzoyl)-8-[(3,3,3-trifluoropropyl)amino]imidazo[1,2-b]pyridazin-3-yl}-2-methylbenzamide,
N-cyclopropyl-4-{6-[1-(3-methoxyphenyl)vinyl]-8-[(3,3,3-trifluoropropyl)amino]imidazo[1,2-b]pyridazin-3-yl}-2-methylbenzamide,
N-cyclopropyl-4-{6-(4-methoxybenzoyl)-8-[(3,3,3-trifluoropropyl)amino]imidazo[1,2-b]pyridazin-3-yl}-2-methylbenzamide,
N-cyclopropyl-4-{6-[1-(4-methoxyphenyl)vinyl]-8-[(3,3,3-trifluoropropyl)amino]imidazo[1,2-b]pyridazin-3-yl}-2-methylbenzamide,
N-cyclopropyl-4-{6-[(2, 5-difluorophenyl) (hydroxy)methyl]-8-[(3,3,3-trifluoropropyl)amino]imidazo[1,2-b]pyridazin-3-yl}-2-methylbenzamide,
N-cyclopropyl-4-{6-(2, 5-difluorobenzoyl)-8-[(3,3,3-trifluoropropyl)amino]imidazo[1,2-b]pyridazin-3-yl}-2-methylbenzamide,
N-cyclopropyl-4-{6-(3-fluoro-4-methoxybenzoyl)-8-[(3,3,3-trifluoropropyl)amino]imidazo[1,2-b]pyridazin-3-yl}-2-methylbenzamide,
N-cyclopropyl-4-{6-(2,3-difluorobenzoyl)-8-[(3,3,3-trifluoropropyl)amino]imidazo[1,2-b]pyridazin-3-yl}-2-methylbenzamide,
N-cyclopropyl-4-{6-[1-(2,3-difluorophenyl)vinyl]-8-[(3,3,3-trifluoropropyl)amino]imidazo[1,2-b]pyridazin-3-yl}-2-methylbenzamide,
N-cyclopropyl-4-{6-[difluoro(4-methoxyphenyl)methyl]-8-[(3,3,3-trifluoropropyl)amino]imidazo[1,2-b]pyridazin-3-yl}-2-methylbenzamide,
N-cyclopropyl-4-{6-[1-(2,3-difluorophenyl)cyclopropyl]-8-[(3,3,3-trifluoropropyl)amino]imidazo[1,2-b]pyridazin-3-yl}-2-methylbenzamide,
N-cyclopropyl-4-{6-[(2,3-difluorophenyl)(difluoro)methyl]-8-[(3,3,3-trifluoropropyl)amino]imidazo[1,2-b]pyridazin-3-yl}-2-methylbenzamide,
N-cyclopropyl-4-{6-[1-(2, 5-difluorophenyl)vinyl]-8-[(3,3,3-trifluoropropyl)amino]imidazo[1,2-b]pyridazin-3-yl}-2-methylbenzamide,
N-cyclopropyl-4-{6-[1-(2, 5-difluorophenyl)cyclopropyl]-8-[(3,3,3-trifluoropropyl)amino]imidazo[1,2-b]pyridazin-3-yl}-2-methylbenzamide,
N-cyclopropyl-4-{6-[(2, 5-difluorophenyl)(difluoro)methyl]-8-[(3,3,3-trifluoropropyl)amino]imidazo[1,2-b]pyridazin-3-yl}-2-methylbenzamide,
N-cyclopropyl-4-{6-[1-(5-fluoro-2-hydroxyphenyl)ethenyl]-8-[(3,3,3-trifluoropropyl)amino]imidazo[1,2-b]pyridazin-3-yl}-2-methylbenzamide,
N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide,
N-cyclopropyl-4-{6-(3-fluoro-4-methoxyphenoxy)-8-[(oxetan-3-ylmethyl)amino]imidazo[1,2-b]pyridazin-3-yl}-2-methylbenzamide,
N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide, and
N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide,
   or an N-oxide, a hydrate, a solvate, or a salt thereof, or a mixture of same.

In a preferred embodiment, the Mps-1 kinase inhibitor is selected from the group consisting of:
N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide,
N-cyclopropyl-4-{6-(3-fluoro-4-methoxyphenoxy)-8-[(oxetan-3-ylmethyl)amino]imidazo[1,2-b]pyridazin-3-yl}-2-methylbenzamide,
N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide, and
N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide,
   or an N-oxide, a hydrate, a solvate, or a salt thereof, or a mixture of same.

In another preferred embodiment, the Mps-1 kinase inhibitor is
N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide,
   or an N-oxide, a hydrate, a solvate, or a salt thereof.

In another preferred embodiment, the Mps-1 kinase inhibitor is N-cyclopropyl-4-{6-(3-fluoro-4-methoxyphenoxy)-8-[(oxetan-3-ylmethyl)amino]imidazo[1,2-b]pyridazin-3-yl}-2-methylbenzamide, or an N-oxide, a hydrate, a solvate, or a salt thereof.

In another preferred embodiment, the Mps-1 kinase inhibitor is N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide, or an N-oxide, a hydrate, a solvate, or a salt thereof.

In another preferred embodiment, the Mps-1 kinase inhibitor is N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide, or an N-oxide, a hydrate, a solvate, or a salt thereof.

The Mps-1 kinase inhibitor can exist as a hydrate, or as a solvate, wherein the Mps-1 kinase inhibitor contains polar solvents, in particular water, methanol or ethanol for example as structural element of the crystal lattice of the compound. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Further, the Mps-1 kinase inhibitor can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of the Mps-1 kinase inhibitor. For example, see S. M. Berge, et al. “Pharmaceutical Salts,” J. Pharm. Sci. 1977, 66, 1-19.

Further, the Mps-1 kinase inhibitor can exist as an N-oxide, which is defined in that at least one nitrogen of the compound is oxidised. The present invention includes all such possible N-oxides.

Furthermore, the present invention includes all possible crystalline forms, or polymorphs, of the Mps-1 kinase inhibitor, either as single polymorphs, or as a mixture of more than one polymorphs, in any ratio.

In summary, the present invention also relates to useful forms of an Mps-1 kinase inhibitor as disclosed herein, such as metabolites, hydrates, solvates, prodrugs, salts, in particular pharmaceutically acceptable salts, and co-precipitates.

The Mps-1 kinase inhibitor and any useful form of the Mps-1 kinase inhibitor as disclosed herein are also referred to as compound A.

The combination according to the invention further comprises one or more mitotic inhibitors.

The mitotic inhibitor hereinafter is also referred to as compound B.

In a preferred embodiment of the invention, the mitotic inhibitor is a vinca alkaloid, including vinblastine, vincristine, vindesine, vinorelbine, desoxyvincaminol, vincaminol, vinburnine, vincamajine, vineridine, and vinburnine.

In a more preferred embodiment, the mitotic inhibitor is selected from the group consisting of vinblastine, vincristine, vindesine, and vinorelbine.

In an even more preferred embodiment, the mitotic inhibitor is vinorelbine.

In another preferred embodiment of the invention, the mitotic inhibitor is a taxane, including docetaxel, paclitaxel, and their analogues.

Taxanes are known in the art and include, for example, paclitaxel, docetaxel, and the like.

### Paclitaxel:

(2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate; commercial names: Taxol, Anzatax, Paxene.

### Docetaxel:

1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}; commercial name: Taxotere.

Taxane based cancer therapy regimens are broadly used in the treatment of ovarian, breast cancer, non-small cell and small cell lung carcinoma, head and neck cancer, esophageal cancer, prostate cancer, bladder cancer and AIDS-related Kaposi's sarcoma. Taxanes, which include paclitaxel, docetaxel and their analogues, are antimicrotubule agents, inhibit microtubule structures within the cell and ultimately cause cell death. Specifically, taxanes such as paclitaxel bind and stabilize microtubules, cause cells to arrest in mitosis and result in cytostatic or cytotoxic responses (E. Chu, et al., ed Cancer Chemotherapy Drug Manual (2010) Jones and Bartlette Publishers.

Other taxanes that become approved by the U.S. Food and Drug Administration (FDA) or foreign counterparts thereof are also preferred for use in the methods and combinations of the present invention. Other taxanes that can be used in the present invention include those described, for example, in 10th NCI-EORTC Symposium on New Drugs in Cancer Therapy, Amsterdam, page 100, Nos. 382 and 383 (Jun. 16-19, 1998); and U.S. Pat. Nos. 4,814,470, 5,721,268, 5,714,513, 5,739,362, 5,728,850, 5,728,725, 5,710,287, 5,637,484, 5,629,433, 5,580,899, 5,549,830, 5,523,219, 5,281,727, 5,939,567, 5,703,117, 5,480,639, 5,250,683, 5,700,669, 5,665,576, 5,618,538, 5,279,953, 5,243,045, 5,654,447, 5,527,702, 5,415,869, 5,279,949, 5,739,016, 5,698,582, 5,478,736, 5,227,400, 5,516,676, 5,489,601, 5,908,759, 5,760,251, 5,578,739, 5,547,981, 5,547,866, 5,344,775, 5,338,872, 5,717,115, 5,620,875, 5,284,865, 5,284,864, 5,254,703, 5,202,448, 5,723,634, 5,654,448, 5,466,834, 5,430,160, 5,407,816, 5,283,253, 5,719,177, 5,670,663, 5,616,330, 5,561,055, 5,449,790, 5,405,972, 5,380,916, 5,912,263, 8,808,113, 5,703,247, 5,618,952, 5,367,086, 5,200,534, 5,763,628, 5,705,508, 5,622,986, 5,476,954, 5,475,120, 5,412,116, 5,916,783, 5,879,929, 5,861,515, 5,795,909, 5,760,252, 5,637,732, 5,614,645, 5,599,820, 5,310,672, RE 34,277, U.S. Pat. Nos. 5,877,205, 5,808,102, 5,766,635, 5,760,219, 5,750,561, 5,637,723, 5,475,011, 5,256,801, 5,900,367, 5,869,680, 5,728,687, 5,565,478, 5,411,984, 5,334,732, 5,919,815, 5,912,264, 5,773,464, 5,670,673, 5,635,531, 5,508,447, 5,919,816, 5,908,835, 5,902,822, 5,880,131, 5,861,302, 5,850,032, 5,824,701, 5,817,867, 5,811,292, 5,763,477, 5,756,776, 5,686,623, 5,646,176, 5,621,121, 5,616,739, 5,602,272, 5,587,489, 5,567,614, 5,498,738, 5,438,072, 5,403,858, 5,356,928, 5,274,137, 5,019,504, 5,917,062, 5,892,063, 5,840,930, 5,840,900, 5,821,263, 5,756,301, 5,750,738, 5,750,562, 5,726,318, 5,714,512, 5,686,298, 5,684,168, 5,681,970, 5,679,807, 5,648,505, 5,641,803, 5,606,083, 5,599,942, 5,420,337, 5,407,674, 5,399,726, 5,322,779, 4,924,011, 5,939,566, 5,939,561, 5,935,955, 5,919,455, 5,854,278, 5,854,178, 5,840,929, 5,840,748, 5,821,363, 5,817,321, 5,814,658, 5,807,888, 5,792,877, 5,780,653, 5,770,745, 5,767,282, 5,739,359, 5,726,346, 5,717,103, 5,710,099, 5,698,712, 5,683,715, 5,677,462, 5,670,653, 5,665,761, 5,654,328, 5,643,575, 5,621,001, 5,608,102, 5,606,068, 5,587,493, 5,580,998, 5,580,997, 5,576,450, 5,574,156, 5,571,917, 5,556,878, 5,550,261, 5,539,103, 5,532,388, 5,470,866, 5,453,520, 5,384,399, 5,364,947, 5,350,866, 5,336,684, 5,296,506, 5,290,957, 5,274,124, 5,264,591, 5,250,722, 5,229,526, 5,175,315, 5,136,060, 5,015,744, 4,924,012, 6,118,011, 6,114,365, 6,107,332, 6,072,060, 6,066,749, 6,066,747, 6,051,724, 6,051,600, 6,048,990, 6,040,330, 6,030,818, 6,028,205, 6,025,516, 6,025,385, 6,018,073, 6,017,935, 6,011,056, 6,005,138, 6,005,138, 6,005,120, 6,002,023, 5,998,656, 5,994,576, 5,981,564, 5,977,386, 5,977,163, 5,965,739, 5,955,489, 5,939,567, 5,939,566, 5,919,815, 5,912,264, 5,912,263, 5,908,835, and 5,902,822, the disclosures of which are incorporated by reference herein in their entirety.

Other compounds that can be used in the invention are those that act through a taxane mechanism. Compounds that act through a taxane mechanism include compounds that have the ability to exert microtubule-stabilizing effects and cytotoxic activity against rapidly proliferating cells, such as tumor cells or other hyperproliferative cellular diseases. Such compounds include, for example, epothilone compounds, such as, for example, epothilone A, B, C, D, E and F, and derivatives thereof. Other compounds that act through a taxane mechanism (e.g., epothilone compounds) that become approved by the FDA or foreign counterparts thereof are also preferred for use in the methods and combinations of the present invention. Epothilone compounds and derivatives thereof are known in the art and are described, for example, in U.S. Pat. Nos. 6,121,029, 6,117,659, 6,096,757, 6,043,372, 5,969,145, and 5,886,026; and WO 97/19086, WO 98/08849, WO 98/22461, WO 98/25929, WO 98/38192, WO 99/01124, WO 99/02514, WO 99/03848, WO 99/07692, WO 99/27890, and WO 99/28324, the disclosures of which are incorporated herein by reference in their entirety.

In a preferred embodiment, the taxane is paclitaxel. In another preferred embodiment, the taxane is docetaxel.

The combination of the present invention may comprise one or more further pharmaceutical agents. In a preferred embodiment, the combination of the present invention further comprises cisplatin.

Further, the present invention relates to:

a kit comprising:
a combination of:
   component A: one or more Mps-1 kinase inhibitors, as described supra, or a physiologically acceptable salt, solvate, or hydrate thereof;
   and
   component B: one or more mitotic inhibitors, including docetaxel, paclitaxel, vinblastine, vincristine, vindesine, and vinorelbine;
   and, optionally, one or more further pharmaceutical agents C;
   in which optionally either or both of said components A and B are in the form of a pharmaceutical formulation which is ready for use to be administered simultaneously, concurrently, separately or sequentially.

Either or both of components A and B of any of the combinations of the present invention may be in a useful form, such as pharmaceutically acceptable salts, co-precipitates, metabolites, hydrates, solvates and prodrugs of all the compounds of examples.

The components may be administered independently of one another by the oral, intravenous, topical, local installations, intraperitoneal or nasal route.

The Mps-1 kinase inhibitor is preferably administered orally. The taxane is preferably administered intravenously. The vinca alkaloid is preferably administered intravenously.

Components A and/or B usually are administered in the form of a pharmaceutical composition that is comprised of a pharmaceutically acceptable carrier and a pharmaceutically effective amount of a compound A and/or a compound B of the present invention.

Conventional procedures for preparing such compositions in appropriate dosage forms can be utilized. Ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

The combinations of the present invention can be used for the treatment or prophylaxis of cancer.

In a preferred embodiment, the combinations of the present invention are used for the treatment of pancreatic cancer.

In another preferred embodiment, the combinations of the present invention are used for the treatment of glioblastoma.

In another preferred embodiment, the combinations of the present invention are used for the treatment of non-small cell lung carcinoma.

In another preferred embodiment, the combinations of the present invention are used for the treatment of ovarian cancer.

In another preferred embodiment, the combinations of the present invention are used for the treatment of gastric cancer.

In another preferred embodiment, the combinations of the present invention are used for the treatment of breast cancer.

Combinations of the present invention might be utilized to inhibit, block, reduce, decrease, etc., cell proliferation and/or cell division, and/or produce apoptosis.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

The treatment or prohylaxis comprises: administering to a mammal in need thereof, including a human, an amount of a compound A and an amount of compound B of this invention, or a pharmaceutically acceptable salt, isomer, polymorph, metabolite, hydrate, solvate or ester thereof; etc. which is effective to treat the disorder.

Non-small-cell lung carcinoma (NSCLC) is any type of epithelial lung cancer other than small cell lung carcinoma (SCLC). As a class, NSCLCs are relatively insensitive to chemotherapy, compared to small cell carcinoma. When possible, they are primarily treated by surgical resection with curative intent, although chemotherapy is increasingly being used both pre-operatively (neoadjuvant chemotherapy) and post-operatively (adjuvant chemotherapy).

The most common types of NSCLC are squamous cell carcinoma, large cell carcinoma, and adenocarcinoma, but there are several other types that occur less frequently, and all types can occur in unusual histologic variants and as mixed cell-type combinations (“Non-small cell lung cancer treatment—National Cancer Institute”; retrieved 2008-10-19; http://www.cancer.gov/CANCERTOPICS/PDQ/TREATMENT/NON-SMALL-CELL-LUNG/PATIENT).

Lung cancer in never-smokers is almost universally NSCLC, with a sizeable majority being adenocarcinoma.

On relatively rare occasions, malignant lung tumors are found to contain components of both SCLC and NSCLC. In these cases, the tumors should be classified as combined small cell lung carcinoma (c-SCLC), and are (usually) treated like “pure” SCLC.

Breast cancer is a type of cancer originating from breast tissue, most commonly from the inner lining of milk ducts or the lobules that supply the ducts with milk. Cancers originating from ducts are known as ductal carcinomas, while those originating from lobules are known as lobular carcinomas. Breast cancer occurs in humans and other mammals. While the overwhelming majority of human cases occur in women, male breast cancer can also occur. Examples of breast cancer include, but are not limited to invasive ductal carcinoma, invasive lobular carcinoma, ductal carcinoma in situ, and lobular carcinoma in situ.

Ovarian cancer is a cancerous growth arising from the ovary. Most (more than 90%) ovarian cancers are classified as “epithelial” and are believed to arise from the surface (epithelium) of the ovary. However, some evidence suggests that the fallopian tube could also be the source of some ovarian cancers. Since the ovaries and tubes are closely related to each other, it is thought that these fallopian cancer cells can mimic ovarian cancer. Other types may arise from the egg cells (germ cell tumor) or supporting cells.

Gastric cancer, also known as stomach cancer, affects the stomach, which is found in the upper part of the abdomen and just below the ribs. The stomach is part of the body's digestive system. It produces acids and enzymes that break down food before passing it to the small intestine. The cancer can develop in any part of the stomach and spread up towards the esophagus (the tube that connects mouth to the stomach) or down into the small intestine.

Glioblastoma multiforme (GBM), WHO classification name “glioblastoma”, is the most common and most aggressive malignant primary brain tumor in humans, involving glial cells.

Pancreatic cancer is a malignant neoplasm originating from transformed cells arising in tissues forming the pancreas. The most common type of pancreatic cancer is adenocarcinoma (tumors exhibiting glandular architecture on light microscopy) arising within the exocrine component of the pancreas. A minority arise from islet cells, and are classified as neuroendocrine tumors.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredients to be administered will generally range from about 0.001 mg/kg to about 200 mg/kg body weight per day, and preferably from about 0.01 mg/kg to about 20 mg/kg body weight per day. Clinically useful dosing schedules of a compound will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

The following Table lists the abbreviations used in this paragraph, and in the Examples section. NMR peak forms are stated as they appear in the spectra, possible higher order effects have not been considered.

| Abbreviation | Meaning |
|---|---|
| EDC | 1-Ethyl-3-(3-dimethyllaminopropyl)carbodiimide |
| DCM | dichloromethane |
| DIPEA | N,N-diisopropylethylamine |
| DMF | N,N-Dimethylformamide |
| DMSO | Dimethyl sulfoxide |
| Pd(dppf)Cl₂ | Dichloro[1,1′-bis(diphenylphosphino)ferrocene]palladium(II) |
| P(oTol)₃ | tri-o-tolylphosphine |
| NMR | nuclear magnetic resonance spectroscopy |
| rt | Room temperature |
| RT | Retention time in minutes |
| MW | molecular weight |
| NMP | N-methylpyrrolidinone |
| Oxone | Potassium peroxymonosulfate |
| UPLC | ultra performance liquid chromatography |

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallisation. In some cases, impurities may be stirred out using a suitable solvent. In some cases, the compounds may be purified by chromatography, particularly flash chromatography, using for example pre-packed silica gel cartridges, e.g. from Separtis such as Isolute® Flash silica gel (silica gel chromatography) or Isolute® Flash NH2 silica gel (aminophase-silica-gel chromatography) in combination with a suitable chromatographic system such as a Flashmaster II (Separtis) or an Isolera system (Biotage) and eluents such as, for example, gradients of hexane/ethyl acetate or DCM/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluants such as, for example, gradients of water and acetonitrile which may contain additives such as trifluoroacetic acid, formic acid or aqueous ammonia.

Analytical UPLC-MS was performed as follows:

Method A: System: UPLC Acquity (Waters) with PDA Detector und Waters ZQ mass spectrometer; Column: Acquity BEH C18 1.7 μm 2.1×50 mm; Temperature: 60° C.; Solvent A: Water+0.1% formic acid; Solvent B: acetonitrile; Gradient: 99% A→1% A (1.6 min)→1% A (0.4 min); Flow: 0.8 mL/min; Injection Volume: 1.0 μl (0.1 mg-1 mg/mL sample concentration); Detection: PDA scan range 210-400 nm—Fixed and ESI (+), scan range 170-800 m/z

### General:

ALL reactions were run under an atmosphere of argon in degassed solvents unless stated otherwise.

### Comparative Example 1

### N-Cyclopropyl-4-{6-(3-fluoro-4-methoxybenzyl)-8-[(3,3,3-trifluoropropyl)amino]imidazo[1,2-b]pyridazin-3-yl}-2-methylbenzamide

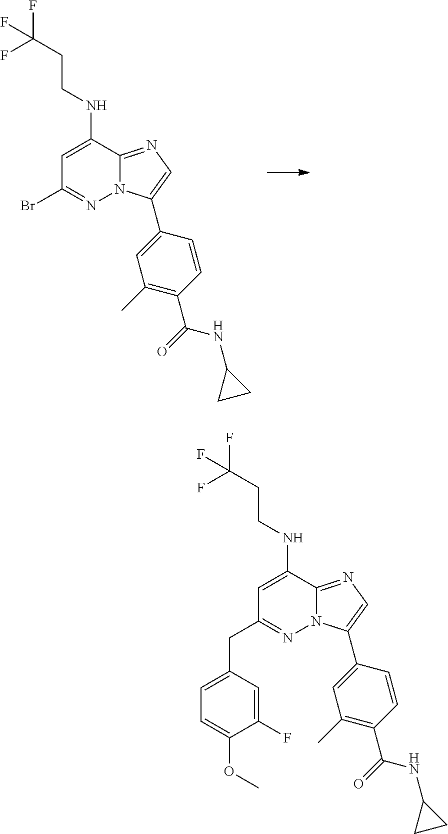

A mixture comprising 300 mg (622 μmol) 4-{6-bromo-8-[(3,3,3-trifluoropropyl)amino]imidazo[1,2-b]pyridazin-3-yl}-N-cyclopropyl-2-methylbenzamide which was prepared according to comparative example 1a, 2.0 mL tetrahydrofuran, 8.29 mL bromo(3-fluoro-4-methoxybenzyl)magnesium (0.75 M in tetrahydrofuran) was stirred at 23° C. overnight. Stirring was continued at 50° C. for 5 hours, the mixture poured into a saturated aqueous ammonium chloride solution. Water was added and the mixture extracted with ethyl acetate. The organic layer was washed with brine and dried over sodium sulfate. After filtration and removal of the solvent, the residue was purified by chromatography to give 261 mg (77%) of the title compound.

¹H-NMR (DMSO-d₆): δ=0.50 (2H), 0.65 (2H), 2.32 (3H), 2.56-2.72 (2H), 2.80 (1H), 3.53 (2H), 3.76 (3H), 3.96 (2H), 6.20 (1H), 7.04-7.12 (2H), 7.20 (1H), 7.30 (1H), 7.46 (1H), 7.92-7.98 (3H), 8.27 (1H) ppm.

### Comparative Example 1a

### 4-{6-Bromo-8-[(3,3,3-trifluoropropyl)amino]imidazo[1,2-b]pyridazin-3-yl}-N-cyclopropyl-2-methylbenzamide

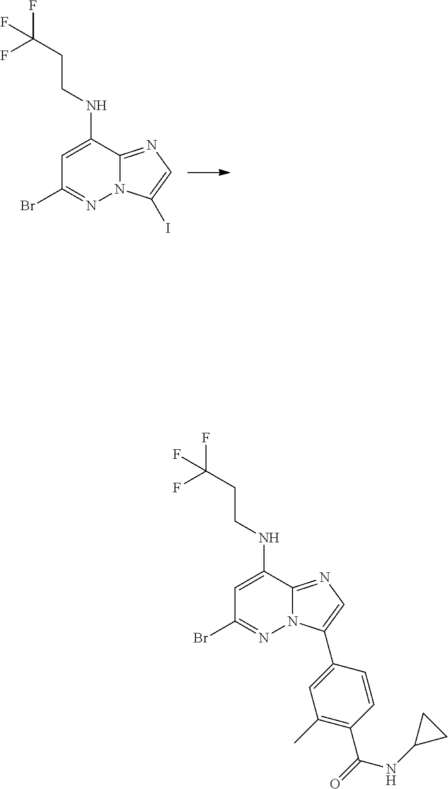 [SMILES: FC(F)(F)CCNc1cc(Br)nn2c(I)cnc12 ; Cc1cc(-c2cnc3c(NCCC(F)(F)F)cc(Br)nn23)ccc1C(=O)NC1CC1]

A mixture comprising 1.00 g (2.3 mmol) 6-bromo-3-iodo-N-(3,3,3-trifluoropropyl)imidazo[1,2-b]pyridazin-8-amine which was prepared according to comparative example 1b, 976 mg N-cyclopropyl-2-methyl-4-(4,4,5,5-tetramethyl-1,3,2-dioxaborolan-2-yl)benzamide which was prepared according to comparative example 1f, 564 mg (1,1,-bis(diphenylphosphino)ferrocene)-dichloropalladium (II), 3.45 mL aqueous 2M cesium carbonate solution and 15 mL tetrahydrofuran was stirred at 45° C. for 12 hours. Water was added and the mixture was extracted with ethyl acetate and methanol. The organic layer was washed with brine and dried over sodium sulfate. After filtration and removal of the solvent the residue was purified by chromatography to give 580 mg (52%) of the title compound.

### Comparative Example 1b

### 6-Bromo-3-iodo-N-(3,3,3-trifluoropropyl)imidazo[1,2-b]pyridazin-8-amine

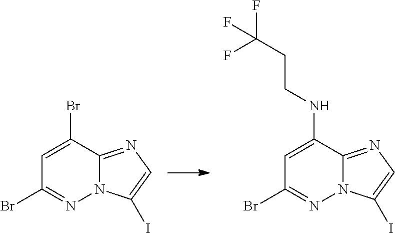 [SMILES: Brc1cc(Br)c2ncc(I)n2n1 ; FC(F)(F)CCNc1cc(Br)nn2c(I)cnc12]

To a solution of 2.30 g (5.71 mmol) 6,8-dibromo-3-iodoimidazo[1,2-b]pyridazine which was prepared according to comparative example 1c in 40 mL N,N-dimethylformamide were added 2.0 g 3,3,3-trifluoropropan-1-amine and the mixture was stirred at 40° C. overnight. Water was added and the mixture was extracted with dichloromethane and methanol. The organic phase was washed with water and dried over sodium sulfate. After filtration and removal of solvent the residue was purified by chromatography to give 2.0 g (81%) of the title compound.

### Comparative Example 1c

### 6,8-Dibromo-3-iodoimidazo[1,2-b]pyridazine

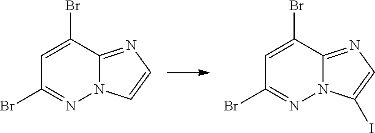 [SMILES: Brc1cc(Br)c2nccn2n1 ; Brc1cc(Br)c2ncc(I)n2n1]

A mixture comprising 3.64 g (10.5 mmol) 6,8-dibromoimidazo[1,2-b]pyridazine which was prepared according to comparative example 1d, 2.8 g N-iodosuccinimide, 72.6 mL N,N-dimethylformamide was heated at 60° C. for 3 hours. 1.4 g N-iodosuccinimide were added and heating was continued for additional 4 hours. Most of the solvent was removed, water was added and the mixture was extracted with dichloromethane. The organic phase was washed with water, sodium thiosulfate solution and dried over sodium sulfate. After filtration and removal of solvent the residue was purified by chromatography to give 3.64 g (86%) of the title compound.

### Comparative Example 1d

### 6,8-Dibromoimidazo[1,2-b]pyridazine

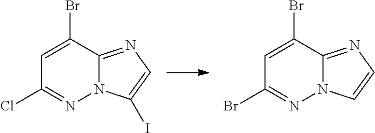 [SMILES: Clc1cc(Br)c2ncc(I)n2n1 ; Brc1cc(Br)c2nccn2n1]

A mixture of 5.0 (14.0 mmol) 8-bromo-6-chloro-3-iodoimidazo[1,2-b]pyridazine which was prepared according to comparative example 1e, 30 mL of hydrogen bromide solution (33% in acetic acid) was stirred at 120° C. for 1 hour under microwave irradiation. The mixture was poured into water and extracted with dichloromethane. The organic phase was washed with sodium thiosulfate and sodium hydrogencarbonate solution and dried over sodium sulfate. After filtration and removal of solvent the residue was purified by chromatography to give 3.0 g (78%) of the title compound.

### Comparative Example 1e

### 8-Bromo-6-chloro-3-iodoimidazo[1,2-b]pyridazine

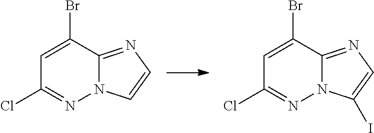 [SMILES: Clc1cc(Br)c2nccn2n1 ; Clc1cc(Br)c2ncc(I)n2n1]

A mixture comprising 100 g (430 mmol) 8-bromo-6-chloroimidazo[1,2-b]pyridazine which was prepared according to a procedure described in US2007/78136 (WO2007/38314), 145 g N-iodosuccinimide, 5 percent per weight conc. hydrochloric acid and 1 L trichloromethane was heated at reflux for 6 hours. 20 g N-iodosuccinimide were added and heating was continued for additional 3 hours. The precipitate was removed and the filtrate was washed with 1N sodium hydroxide solution, brine and dried over sodium sulfate. After filtration and removal of solvent diisopropyl ether was added and the residue was stirred at 23° C. overnight. The precipitate was filtered off and dried to give 66.6 g (43%) of the title compound.

### Comparative Example 1f

### N-Cyclopropyl-2-methyl-4-(4,4,5,5-tetramethyl-1,3,2-dioxaborolan-2-yl)benzamide

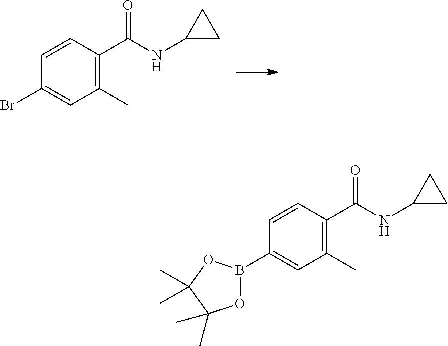 [SMILES: Cc1cc(Br)ccc1C(=O)NC1CC1 ; Cc1cc(B2OC(C)(C)C(C)(C)O2)ccc1C(=O)NC1CC1]

To a solution of 260 g (1.02 mol) 4-bromo-N-cyclopropyl-2-methylbenzamide which was prepared according to comparative example 1g in 2 L dioxane at 23° C. were added 390 g bis-(pinacolato)-diboron, 19.5 g 2-dicyclohexylphosphino-2′,4′,6′-triisopropylbiphenyl, 150 g potassium acetate and 9.37 g tris-(dibenzylidenaceton)-dipalladium(0) and the mixture was refluxed for 6 h. After cooling to 23° C., water and ethyl acetate were added and the mixture stirred for 15 min. The organic phase was washed with water, dried over sodium sulfate, filtered and evaporated. The residue was purified by chromatography to give 308 g (56%) of the title compound.

### Comparative Example 1g

### 4-Bromo-N-cyclopropyl-2-methylbenzamide

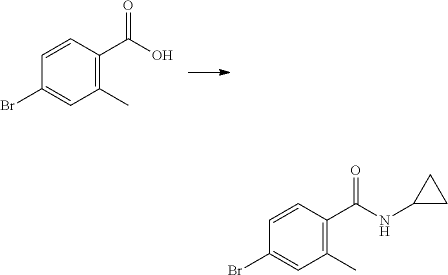

To a stirred solution of 300 g (1.4 mol) 4-bromo-2-methylbenzoic acid in 8.4 L dichloromethane at 23° C. were added 79.6 g cyclopropanamine and 320.9 g EDC. After stirring overnight, the solution was washed with water and the aqueous phase was extracted with dichloromethane. The combined organic phases were dried over sodium sulfate, filtered and evaporated. The remaining solid was triturated with diisopropyl ether, filtered, washed and dried in vacuo to yield 260 g (73%) of the title compound.

### Comparative Example 2

### N-Cyclopropyl-4-{6-(3-fluoro-2-hydroxybenzyl)-8-[(3,3,3-trifluoropropyl)amino]imidazo[1,2-b]pyridazin-3-yl}-2-methylbenzamide

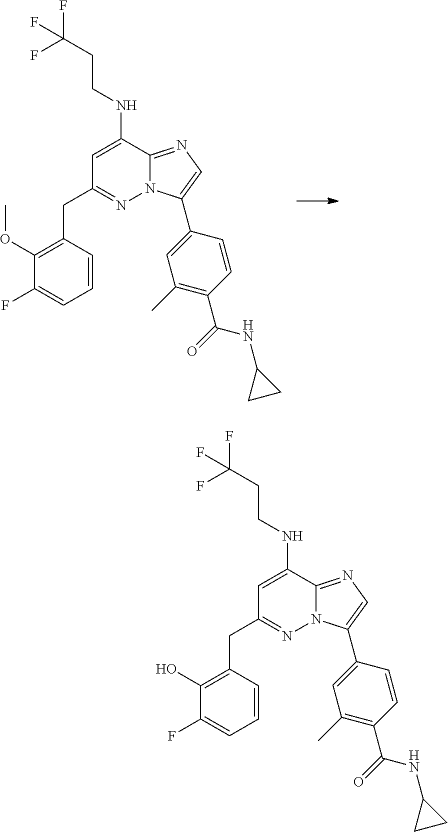

To a solution of 14.2 mg (26 μmol) N-cyclopropyl-4-{6-(3-fluoro-2-methoxybenzyl)-8-[(3,3,3-trifluoropropyl)amino]imidazo[1,2-b]pyridazin-3-yl}-2-methylbenzamide which was prepared according to comparative example 2a in 1 mL dichloromethane were added 131 μL of a 1M boron tribromide solution in dichloromethane and the mixture was stirred at 23° C. for 1 hour. Methanol was added and solvents were removed. The residue was purified by chromatography to give 4.9 mg (32%) of the title compound. UPLC-MS: RT=1.20 min; m/z (ES+) 528.5 [MH⁺]; required MW=527.5.

¹H-NMR (DMSO-d₆): δ=0.49 (2H), 0.65 (2H), 2.29 (3H), 2.56-2.70 (2H), 2.80 (1H), 3.52 (2H), 4.04 (2H), 6.15 (1H), 6.74 (1H), 6.96-7.06 (2H), 7.26 (1H), 7.41 (1H), 7.88-7.96 (3H), 8.23 (1H), 8.70 (1H) ppm.

### Comparative Example 2a

### N-Cyclopropyl-4-{6-(3-fluoro-2-methoxybenzyl)-8-[(3,3,3-trifluoropropyl)amino]imidazo[1,2-b]pyridazin-3-yl}-2-methylbenzamide

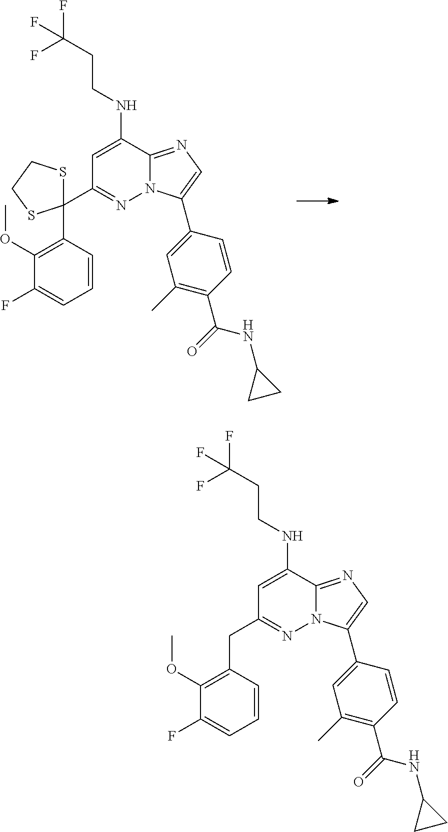

A mixture comprising 30 mg (48 μmol) N-cyclopropyl-4-{6-[2-(3-fluoro-2-methoxyphenyl)-1,3-dithiolan-2-yl]-8-[(3,3,3-trifluoropropyl)amino]imidazo[1,2-b]pyridazin-3-yl}-2-methylbenzamide which was prepared according to comparative example 2b, 800 μL methanol, 200 μL tetrahydrofuran, 18.8 mg dichloronickel hexahydrate and 15.0 mg sodium borohydride was stirred at 23° C. for 2 hours. After filtration water was added and the mixture extracted with ethyl acetate. The organic layer was washed with water and dried over sodium sulfate. After filtration and removal of the solvent, 24.2 mg (93%) of the title compound were obtained that was used without further purification. UPLC-MS: RT=1.30 min; m/z (ES+) 542.6 [MH⁺]; required MW=541.6.

### Comparative Example 2b

### N-Cyclopropyl-4-{6-[2-(3-fluoro-2-methoxyphenyl)-1,3-dithiolan-2-yl]-8-[(3,3,3-trifluoropropyl)amino]imidazo[1,2-b]pyridazin-3-yl}-2-methylbenzamide

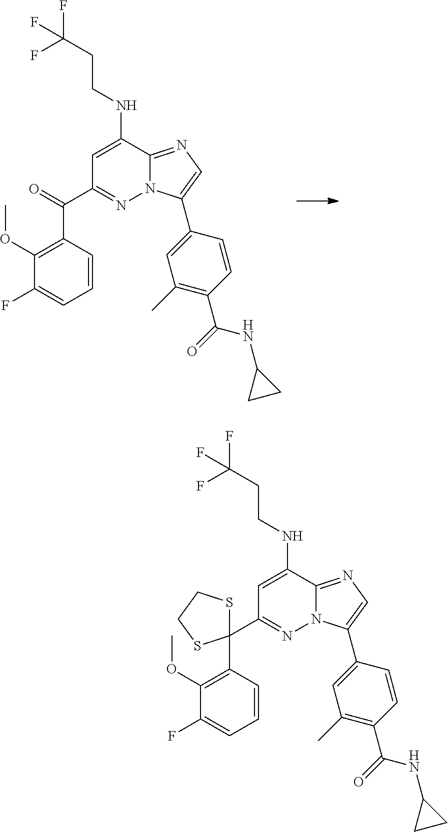

A mixture comprising 150 mg (270 μmol) N-cyclopropyl-4-{6-(3-fluoro-2-methoxybenzoyl)-8-[(3,3,3-trifluoropropyl)amino]imidazo[1,2-b]pyridazin-3-yl}-2-methylbenzamide which was prepared according to comparative example 2c, 340 μL ethane-1,2-dithiol and 37.5 μL boron trifluoride acetic acid complex was heated at 60° C. for 16 hours. Ethyl acetate was added and the mixture washed with saturated sodium hydrogen carbonate, sodium hydroxide solution (1M) and brine. The organic layer was dried over sodium sulfate. After filtration and removal of the solvent, the residue was purified by chromatography to give 63.0 mg (37%) of the title compound. UPLC-MS: RT=1.37 min; m/z (ES+) 632.7 [MH⁺]; required MW=631.7.

¹H-NMR (DMSO-d₆): δ=0.44-0.51 (2H), 0.59-0.68 (2H), 2.58-2.72 (3H), 2.77 (1H), 3.13 (2H), 3.32-3.40 (2H), 3.42 (3H), 3.50-3.69 (4H), 6.71 (1H), 7.06 (1H), 7.17 (1H), 7.23-7.33 (1H), 7.53-7.60 (2H), 7.69 (1H), 7.83 (1H), 8.00 (1H), 8.19 (1H) ppm.

### Comparative Example 2c

### N-Cyclopropyl-4-{6-(3-fluoro-2-methoxybenzoyl)-8-[(3,3,3-trifluoropropyl)amino]imidazo[1,2-b]pyridazin-3-yl}-2-methylbenzamide

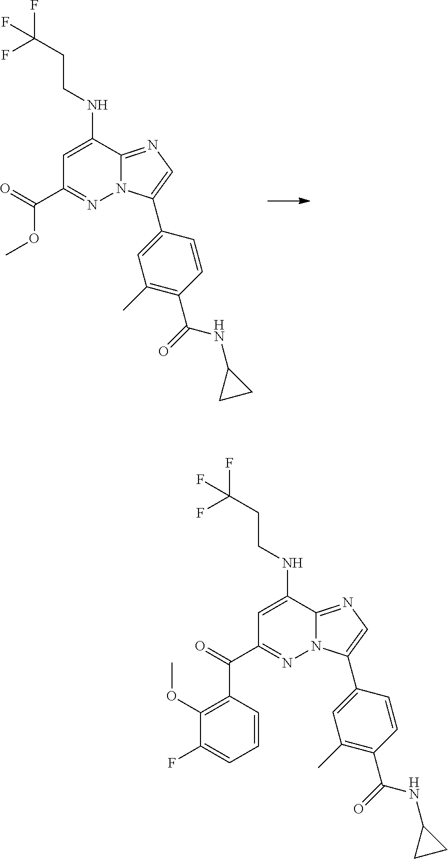 [SMILES: COC(=O)c1cc(NCCC(F)(F)F)c2ncc(-c3ccc(C(=O)NC4CC4)c(C)c3)n2n1 ; COc1c(F)cccc1C(=O)c1cc(NCCC(F)(F)F)c2ncc(-c3ccc(C(=O)NC4CC4)c(C)c3)n2n1]

A mixture comprising 460 mg (997 mmol) methyl 3-[4-(cyclopropylcarbamoyl)-3-methylphenyl]-8-[(3,3,3-trifluoropropyl)amino]imidazo[1,2-b]pyridazine-6-carboxylate which was prepared according to comparative example 2d, 10 mL tetrahydrofuran and 126 mg N-methoxymethanamine hydrochloride was cooled to −5° C. 35.9 mL bromo(3-fluoro-2-methoxyphenyl)magnesium solution in tetrahydrofuran (0.5 M) were added, the mixture stirred at 23° C. overnight and poored into cold hydrochloric acid. Ethyl acetate was added and the mixture washed with brine. The organic layer was dried over sodium sulfate. After filtration and removal of the solvent, the residue was purified by chromatography to give 306 mg (55%) of the title compound. UPLC-MS: RT=1.30 min; m/z (ES+) 556.5 [MH⁺]; required MW=555.5.

¹H-NMR (DMSO-d₆): δ=0.44-0.51 (2H), 0.59-0.70 (2H), 2.10 (3H), 2.64-2.83 (3H), 3.62 (3H), 3.72 (2H), 6.79 (1H), 7.00-7.06 (1H), 7.14 (1H), 7.26 (1H), 7.50 (1H), 7.54 (1H), 7.71 (1H), 7.82 (1H), 7.94 (1H), 8.22 (1H) ppm.

### Comparative Example 2d

### Methyl 3-[4-(cyclopropylcarbamoyl)-3-methylphenyl]-8-[(3,3,3-trifluoropropyl)amino]imidazo[1, 2-b]pyridazine-6-carboxylate

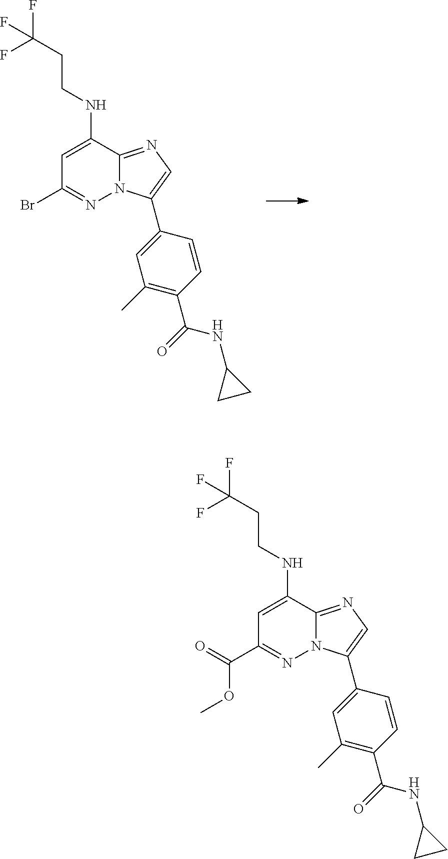 [SMILES: Cc1cc(-c2cnc3c(NCCC(F)(F)F)cc(Br)nn23)ccc1C(=O)NC1CC1 ; COC(=O)c1cc(NCCC(F)(F)F)c2ncc(-c3ccc(C(=O)NC4CC4)c(C)c3)n2n1]

A mixture comprising 5.0 g (10.37 mmol) 4-{6-bromo-8-[(3,3,3-trifluoropropyl)amino]imidazo[1,2-b]pyridazin-3-yl}-N-cyclopropyl-2-methylbenzamide which was prepared according to comparative example 1a, 100 mL methanol, 10 mL tetrahydrofuran, 1.7 g (1,1,-bis(diphenylphosphino)ferrocene)-dichloropalladium (II), 1.6 mL triethylamine was reacted under an atmosphere of carbon monoxide at 100° C., 9-12 bar for 24 hours. After removal of the solvents, the residue was purified by chromatography to give 3.32 g (63%) of the title compound. UPLC-MS: RT=1.11 min; m/z (ES+) 462.5 [MH⁺]; required MW=461.5.

### Comparative Example 3

### N-Cyclopropyl-4-{6-(3-fluorobenzyl)-8-[(3,3,3-trifluoropropyl)amino]imidazo[1, 2-b]pyridazin-3-yl}-2-methylbenzamide

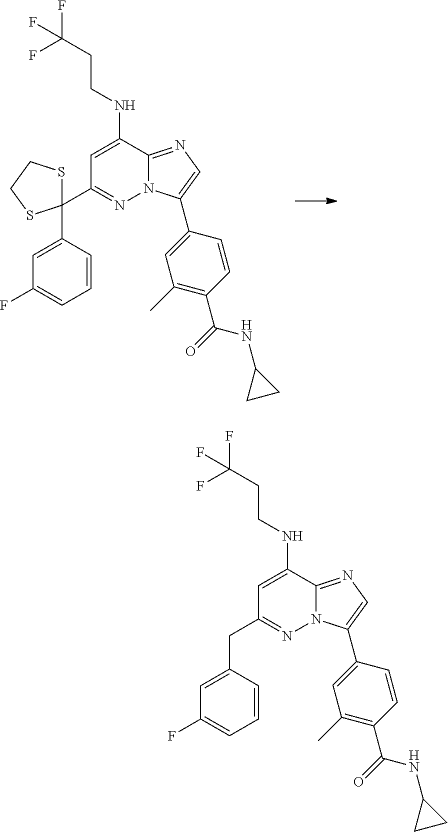

45 mg (75 μmol) N-cyclopropyl-4-{6-[2-(3-fluorophenyl)-1,3-dithiolan-2-yl]-8-[(3,3,3-trifluoropropyl)amino]imidazo[1,2-b]pyridazin-3-yl}-2-methylbenzamide which was prepared according to comparative example 3a were transformed in analogy to comparative example 2a to give after working up and purification 16.3 mg (42%) of the title compound. UPLC-MS: RT=1.30 min; m/z (ES+) 556.5 [MH⁺]; required MW=555.5.

¹H-NMR (DMSO-d₆): δ=0.50 (2H), 0.65 (2H), 2.32 (3H), 2.56-2.72 (2H), 2.80 (1H), 3.54 (2H), 4.05 (2H), 6.22 (1H), 7.03 (1H), 7.15-7.23 (2H), 7.26-7.38 (2H), 7.46 (1H), 7.91-7.99 (3H), 8.24 (1H) ppm.

### Comparative Example 3a

### N-Cyclopropyl-4-{6-[2-(3-fluorophenyl)-1, 3-dithiolan-2-yl]-8-[(3,3,3-trifluoropropyl)amino]imidazo[1,2-b]pyridazin-3-yl}-2-methylbenzamide

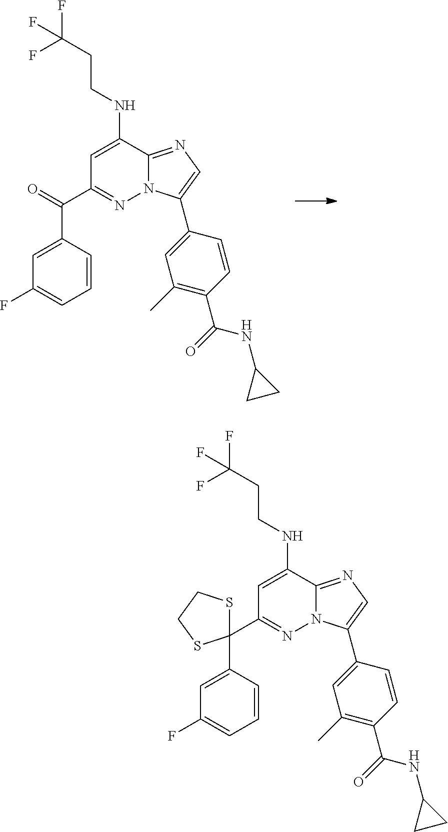

80 mg (152 μmol) N-cyclopropyl-4-{6-(3-fluorobenzoyl)-8-[(3,3,3-trifluoropropyl)amino]imidazo[1,2-b]pyridazin-3-yl}-2-methylbenzamide which was prepared according to comparative example 3b were transformed in analogy to comparative example 2b to give after working up and purification 45 mg (49%) of the title compound. UPLC-MS: RT=1.39 min; m/z (ES+) 602.7 [MH⁺]; required MW=601.7.

¹H-NMR (DMSO-d₆): δ=0.46-0.53 (2H), 0.61-0.68 (2H), 2.30 (3H), 2.52-2.65 (2H), 2.76-2.85 (1H), 3.32-3.41 (2H), 3.48-3.62 (4H), 6.26 (1H), 7.06-7.13 (1H), 7.26 (1H), 7.34 (1H), 7.39-7.43 (1H), 7.48 (1H), 7.63 (1H), 7.91 (1H), 8.05 (2H), 8.25 (1H) ppm.

### Comparative Example 3b

N-Cyclopropyl-4-{6-(3-fluorobenzoyl)-8-[(3,3,3-trifluoropropyl)amino]imidazo[1,2-b]pyridazin-3-yl}-2-methylbenzamide

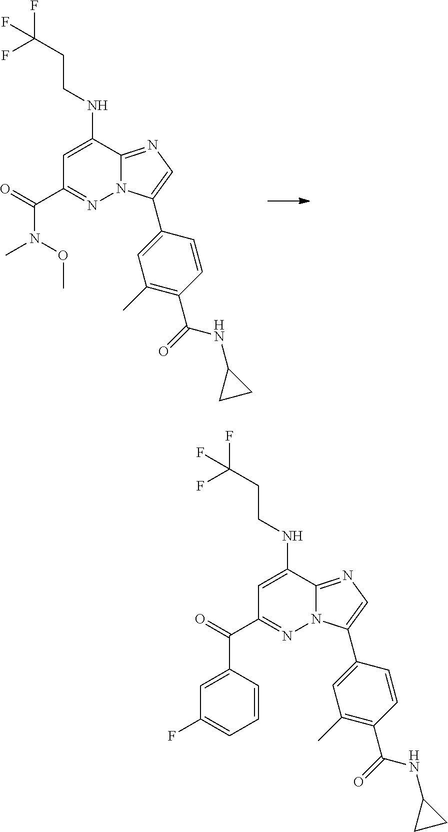

To a solution of 400 mg (0.816 mmol) 3-[4-(cyclopropylcarbamoyl)-3-methylphenyl]-N-methoxy-N-methyl-8-[(3,3,3-trifluoropropyl)amino]imidazo[1,2-b]pyridazine-6-carboxamide which was prepared according to comparative example 3c in 30 mL THF were added 12.23 mL (15 eq) bromo(3-fluorophenyl)magnesium (1M solution in THF) at −20° C. After further 30 min. stirring at this temperature, the solution is added dropwise to 50 mL ice-cold 0.5 M HCL solution to give after working up and purification 334 mg (78%) of the title compound. UPLC-MS: RT=1.32 min; m/z (ES+) 526.5 [MH+]; required MW=525.5.

¹H-NMR (DMSO-d₆): δ=0.44-0.52 (2H), 0.59-0.69 (2H), 2.20 (3H), 2.62-2.84 (3H), 3.70 (2H), 6.74 (1H), 7.10 (1H), 7.24 (1H), 7.51-7.66 (2H), 7.79-7.96 (4H), 8.15 (1H), 8.23 (1H) ppm.

### Comparative Example 3c

### 3-[4-(cyclopropylcarbamoyl)-3-methylphenyl]-N-methoxy-N-methyl-8-[(3,3,3-trifluoropropyl)amino]imidazo[1, 2-b]pyridazine-6-carboxamide

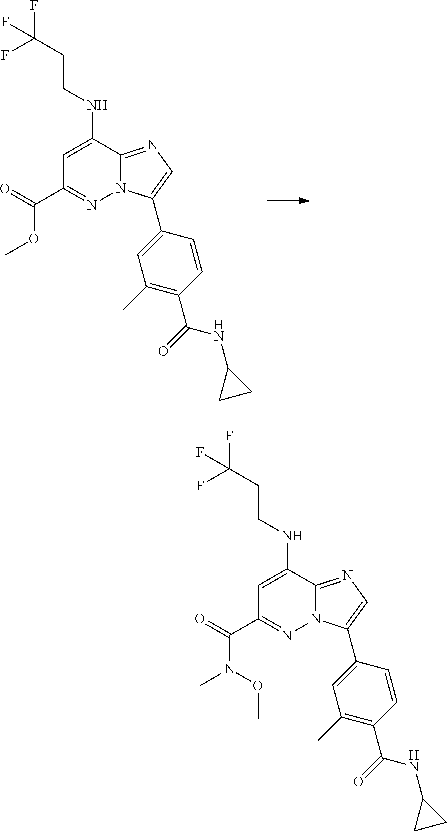

To a suspension of 6.62 g (14.34 mmol) methyl 3-[4-(cyclopropylcarbamoyl)-3-methylphenyl]-8-[(3,3,3-trifluoropropyl)amino]imidazo[1,2-b]pyridazine-6-carboxylate which was prepared according to comparative example 2d and 2.10 g (21.52 mmol) N-methoxymethanamine hydrochloride (1:1) in 30 mL THF were dropwise added 33 mL lithium chloride-chloro(propan-2-yl)magnesium (1:1) (3 eq, 1.3M solution in THF) at −20° C. After 2 h stirring at this temperature, further 55 mL (5 eq) mL lithium chloride-chloro(propan-2-yl)magnesium (1:1) solution were added. After 40 min the reaction is quenched by addition of 20% ammonia chloride solution to give after working up and purification 3.8 g (55%) of the title compound. UPLC-MS: RT=1.05 min; m/z (ES+) 491.5 [MH⁺]; required MW=490.5.

¹H-NMR (DMSO-d₆): δ=0.44-0.53 (2H), 0.60-0.69 (2H), 2.35 (3H), 2.57-2.73 (2H), 2.81 (1H), 3.54-3.69 (5H), 6.36 (1H), 7.36 (1H), 7.81 (1H), 7.90 (1H), 7.95 (1H), 8.04 (1H), 8.28 (1H) ppm. comparative

### Comparative Example 4

### N-cyclopropyl-4-{6-(3-methoxybenzyl)-8-[(3,3,3-trifluoropropyl)amino]imidazo[1,2-b]pyridazin-3-yl}-2-methylbenzamide

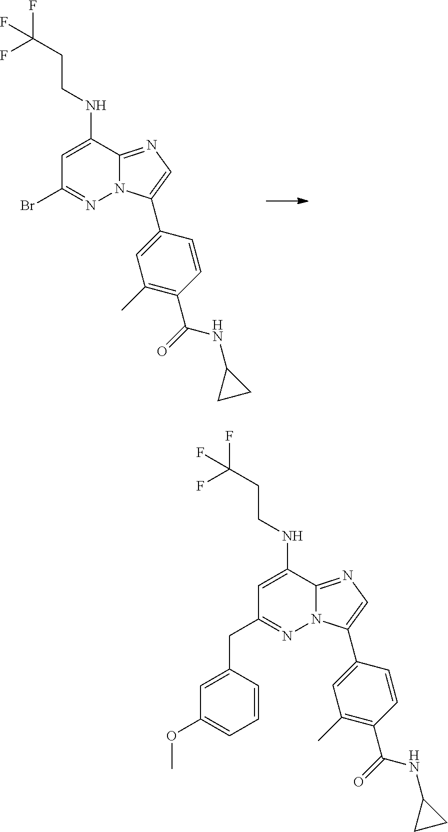

100 mg (207 μmol) 4-{6-bromo-8-[(3,3,3-trifluoropropyl)amino]imidazo[1,2-b]pyridazin-3-yl}-N-cyclopropyl-2-methylbenzamide which was prepared according to comparative example 1a were transformed in analogy to comparative example 1 using bromo(3-methoxybenzyl)magnesium to give after working up and purification 28.7 mg (25%) of the title compound.

¹H-NMR (DMSO-d₆): δ=0.50 (2H), 0.65 (2H), 2.33 (3H), 2.56-2.72 (2H), 2.80 (1H), 3.53 (2H), 3.69 (3H), 3.99 (2H), 6.28 (1H), 6.77 (1H), 6.87-6.97 (2H), 7.20 (1H), 7.32 (1H), 7.54 (1H), 7.93-8.05 (3H), 8.28 (1H) ppm.

### Comparative Example 5

### N-Cyclopropyl-4-{6-(4-methoxybenzyl)-8-[(3,3,3-trifluoropropyl)amino]imidazo[1,2-b]pyridazin-3-yl}-2-methylbenzamide

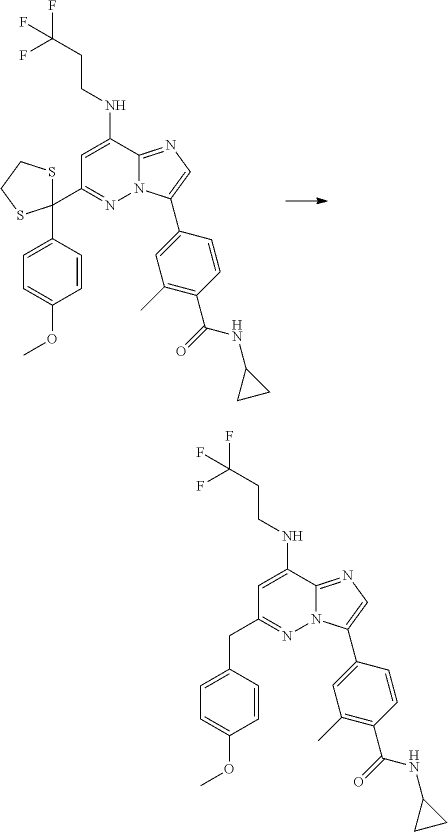

30 mg (49 μmol) N-cyclopropyl-4-{6-[2-(4-methoxyphenyl)-1,3-dithiolan-2-yl]-8-[(3,3,3-trifluoropropyl)amino]imidazo[1,2-b]pyridazin-3-yl}-2-methylbenzamide which was prepared according to comparative example 5a were transformed in analogy to comparative example 2a to give after working up and purification 7.9 mg (29%) of the title compound.

¹H-NMR (DMSO-d₆): δ=0.50 (2H), 0.65 (2H), 2.33 (3H), 2.55-2.71 (2H), 2.80 (1H), 3.52 (2H), 3.68 (3H), 3.94 (2H), 6.16 (1H), 6.85 (2H), 7.25 (2H), 7.31 (1H), 7.43 (1H), 7.91-8.01 (3H), 8.27 (1H) ppm.

### Comparative Example 5a

### N-Cyclopropyl-4-{6-[2-(4-methoxyphenyl)-1,3-dithiolan-2-yl]-8-[(3,3,3-trifluoropropyl)amino]imidazo[1,2-b]pyridazin-3-yl}-2-methylbenzamide

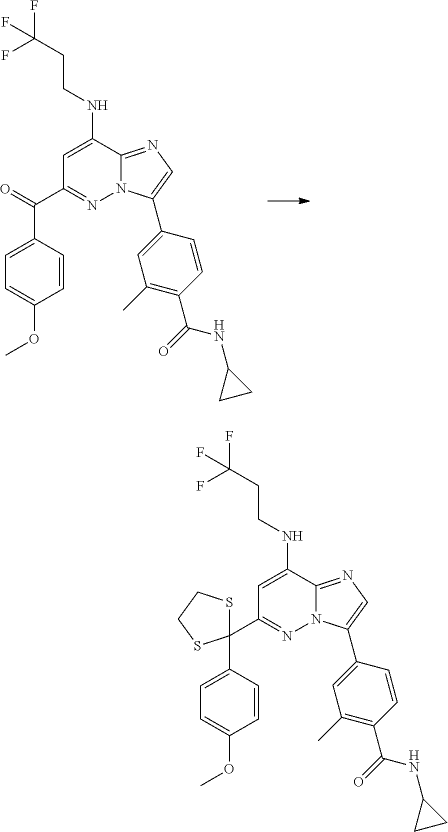

100 mg (186 μmol) N-cyclopropyl-4-{6-(4-methoxybenzoyl)-8-[(3,3,3-trifluoropropyl)amino]imidazo[1,2-b]pyridazin-3-yl}-2-methylbenzamide which was prepared according to example 9 were transformed in analogy to comparative example 2b to give after working up and purification 60.2 mg (53%) of the title compound.

### Compounds of the Present Invention

### Compound A1

### N-Cyclopropyl-4-{6-[1-(3-fluoro-4-methoxyphenyl)ethenyl]-8-[(3, 3,3-trifluoropropyl)amino]imidazo[1, 2-b]pyridazin-3-yl}-2-methylbenzamide

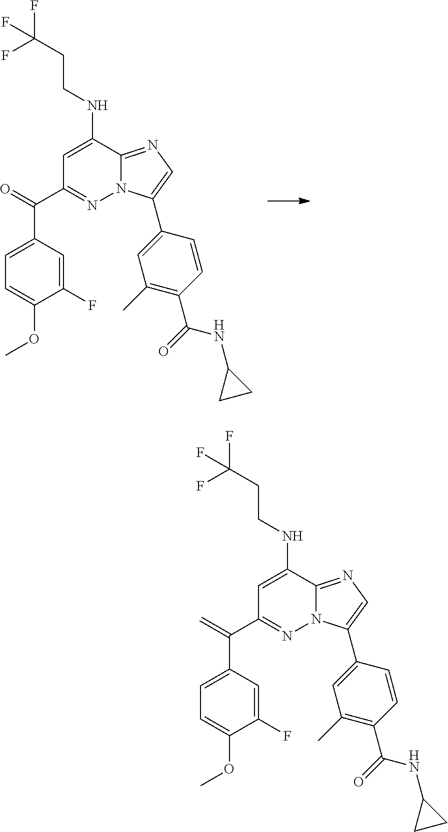

To a suspension of 386 mg methyl(triphenyl)phosphonium bromide in 6.8 mL tetrahydrofuran at −78° C. were added 421 μL n-butyllithium (2.5M in hexane). After the mixture was stirred at 0° C. for 0.5 hours a solution of 150 mg (270 μmol) N-cyclopropyl-4-{6-(3-fluoro-4-methoxybenzoyl)-8-[(3,3,3-trifluoropropyl)amino]imidazo[1,2-b]pyridazin-3-yl}-2-methylbenzamide which was prepared according to intermediate example 1a in 3.2 mL tetrahydrofuran was added and stirring was continued overnight. Water was added and the mixture was extracted with ethyl acetate. The organic layer was washed with saturated ammonium chloride solution and dried over sodium sulfate. After filtration and removal of the solvent the residue was purified by chromatography to give 127 mg (81%) of the title compound.

¹H-NMR (DMSO-d₆): δ=0.48 (2H), 0.64 (2H), 2.17 (3H), 2.61-2.73 (2H), 2.78 (1H), 3.61 (2H), 3.84 (3H), 5.73 (1H), 5.93 (1H), 6.36 (1H), 7.13-7.23 (3H), 7.30 (1H), 7.54 (1H), 7.82 (1H), 7.88 (1H), 8.01 (1H), 8.21 (1H) ppm.

### Intermediate Example 1a

### N-Cyclopropyl-4-{6-(3-fluoro-4-methoxybenzoyl)-8-[(3,3,3-trifluoropropyl)amino]imidazo[1,2-b]pyridazin-3-yl}-2-methylbenzamide

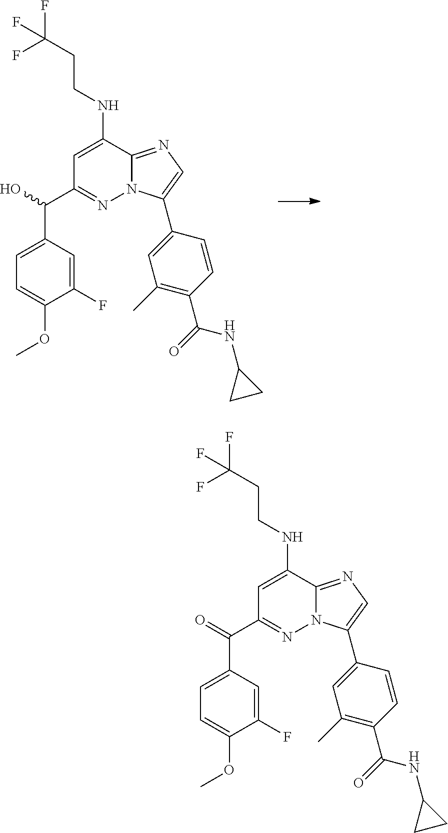

To a solution of 82 μL ethanedioyl dichloride in 2.5 mL dichloromethane were added at −78° C. 133 μL dimethyl sulfoxide followed by a solution of 262 mg (470 μmol) (RS)—N-cyclopropyl-4-{6-[(3-fluoro-4-methoxyphenyl)(hydroxy)methyl]-8-[(3,3,3-trifluoropropyl)amino]imidazo[1,2-b]pyridazin-3-yl}-2-methylbenzamide which was prepared according to intermediate example 1b in 2.5 mL dichloromethane and 0.6 mL dimethyl sulfoxide. After 1 hour, 393 μL triethylamine were added and the mixture was stirred at 23° C. for 20 minutes. Water was added and the mixture was extracted dichloromethane and methanol (9:1). The organic layer was washed with water and dried over sodium sulfate. After filtration and removal of the solvent the residue was purified by chromatography to give 210 mg (80%) of the title compound.

### Intermediate Example 1b

### (RS)—N-Cyclopropyl-4-{6-[(3-fluoro-4-methoxyphenyl)(hydroxy)methyl]-8-[(3,3,3-trifluoropropyl)amino]imidazo[1,2-b]pyridazin-3-yl}-2-methylbenzamide

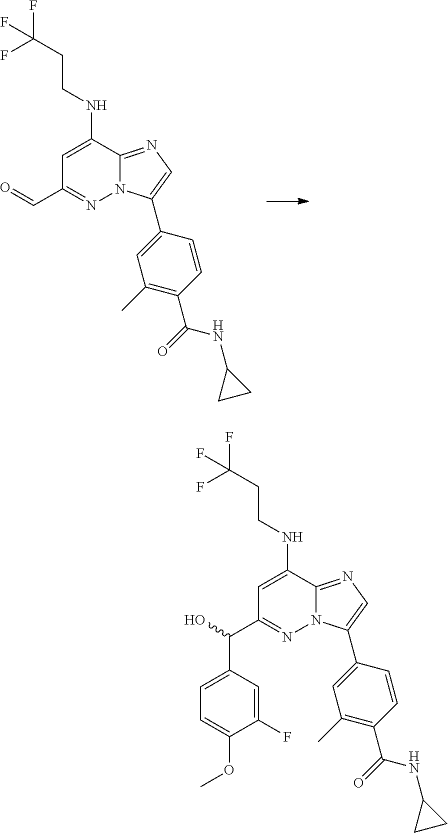

To a solution of 500 mg (1.16 mmol) N-cyclopropyl-4-{6-formyl-8-[(3,3,3-trifluoropropyl)amino]imidazo[1,2-b]pyridazin-3-yl}-2-methylbenzamide which was prepared according to intermediate example 1c in 20 mL tetrahydrofuran were added at 0° C. a solution of bromo(3-fluoro-4-methoxyphenyl)magnesium freshly prepared from 598 μL 4-bromo-2-fluoro-1-methoxybenzene, 113 mg magnesium and 5 mL tetrahydrofuran. After 1 hour the mixture was poured into a saturated aqueous ammonium chloride solution. Water was added and the mixture extracted with ethyl acetate. The organic layer was washed with brine and dried over sodium sulfate. After filtration and removal of the solvent, the residue was purified by chromatography to give 319 mg (46%) of the title compound.

### Intermediate Example 1c

### N-Cyclopropyl-4-{6-formyl-8-[(3,3,3-trifluoropropyl)amino]imidazo[1,2-b]pyridazin-3-yl}-2-methylbenzamide

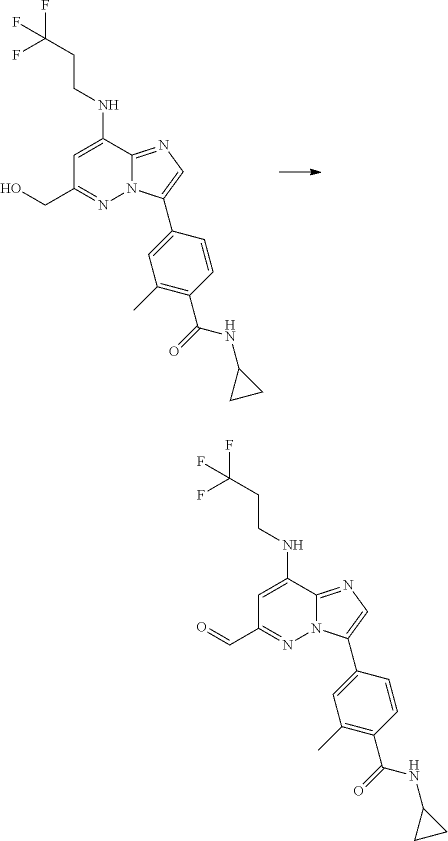

1.60 g (3.69 mmol) N-cyclopropyl-4-{6-(hydroxymethyl)-8-[(3,3,3-trifluoropropyl)amino]imidazo[1,2-b]pyridazin-3-yl}-2-methylbenzamide which was prepared according to intermediate example 1d were transformed in analogy to intermediate example 1a to give after working up and purification 1.50 g (94%) of the title compound.

### Intermediate Example 1d

### N-Cyclopropyl-4-{6-(hydroxymethyl)-8-[(3,3,3-trifluoropropyl)amino]imidazo[1,2-b]pyridazin-3-yl}-2-methylbenzamide

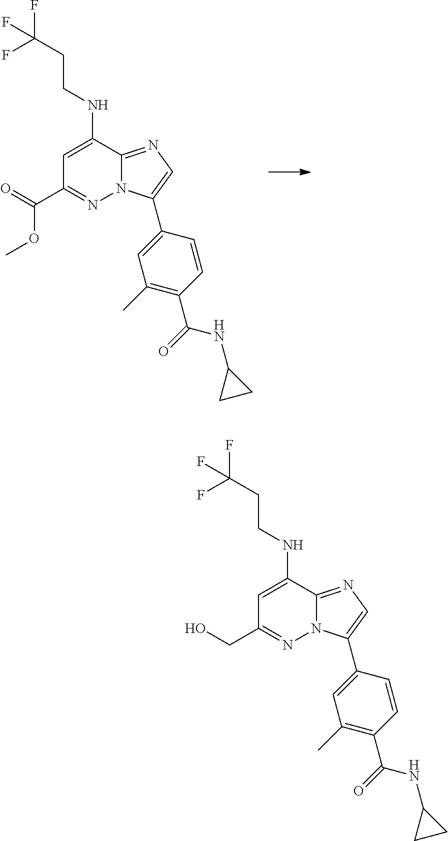

To a solution of 2.17 g (4.70 mmol) methyl 3-[4-(cyclopropylcarbamoyl)-3-methylphenyl]-8-[(3,3,3-trifluoropropyl)amino]imidazo[1,2-b]pyridazine-6-carboxylate which was prepared according to comparative example 2d in 220 mL tetrahydrofuran at 0° C. were added 23.5 mL diisobutylaluminiumhydrid solution (1M in tetrahydrofuran). After 1 hour the mixture was poured into a saturated aqueous ammonium chloride solution. Water was added and the mixture extracted with ethyl acetate and methanol (9:1). The organic layer was washed with brine and dried over sodium sulfate. After filtration and removal of the solvent, the residue was purified by chromatography to give 1.56 g (73%) of the title compound.

### Compound A2

### N-Cyclopropyl-4-{6-[difluoro(3-fluoro-4-methoxyphenyl)methyl]-8-[(3,3,3-trifluoropropyl)amino]imidazo[1,2-b]pyridazin-3-yl}-2-methylbenzamide

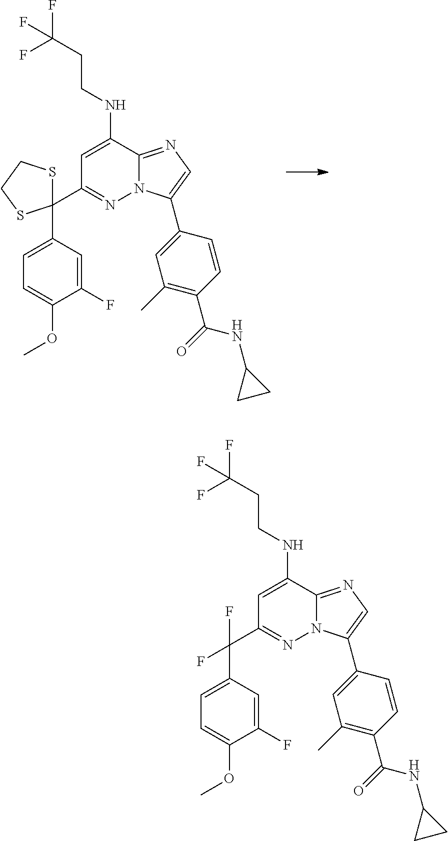

To a mixture of 32.5 mg 1-(chloromethyl)-4-fluoro-1,4-diazoniabicyclo[2.2.2]octane ditetrafluoroborate and 1.16 mL pyridine hydrofluoride at 0° C. was added a solution of 29 mg (46 μmol) N-cyclopropyl-4-{6-[2-(3-fluoro-4-methoxyphenyl)-1,3-dithiolan-2-yl]-8-[(3,3,3-trifluoropropyl)amino]imidazo[1,2-b]pyridazin-3-yl}-2-methylbenzamide which was prepared according to intermediate example 2a in 0.5 mL dichloromethane. The mixture was stirred at 23° C. overnight and poured into water. The organic layer was washed with water and brine and dried over sodium sulfate. After filtration and removal of the solvent, the residue was purified by chromatography to give 14.6 mg (52%) of the title compound.

¹H-NMR (DMSO-d₆): δ=0.49 (2H), 0.65 (2H), 2.23 (3H), 2.61-2.84 (3H), 3.68 (2H), 3.86 (3H), 6.58 (1H), 7.24 (1H), 7.31 (1H), 7.42 (1H), 7.50 (1H), 7.72-7.81 (2H), 7.97 (1H), 8.09 (1H), 8.27 (1H) ppm.

### Intermediate Example 2a

### N-Cyclopropyl-4-{6-[2-(3-fluoro-4-methoxyphenyl)-1,3-dithiolan-2-yl]-8-[(3,3,3-trifluoropropyl)amino]imidazo[1,2-b]pyridazin-3-yl}-2-methylbenzamide

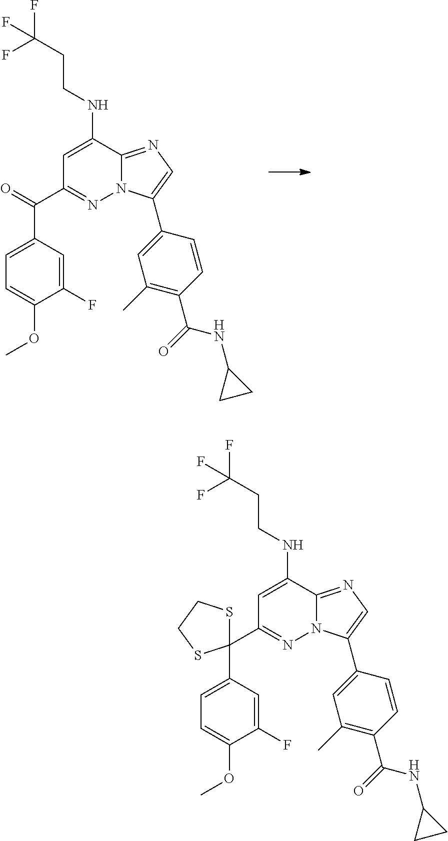

50 mg (90 μmol) N-cyclopropyl-4-{6-(3-fluoro-4-methoxybenzoyl)-8-[(3,3,3-trifluoropropyl)amino]imidazo[1,2-b]pyridazin-3-yl}-2-methylbenzamide which was prepared according to intermediate example 1a were transformed in analogy to comparative example 2b to give after working up and purification 29 mg (51%) of the title compound.

### Compound A3

### (RS)—N-Cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide

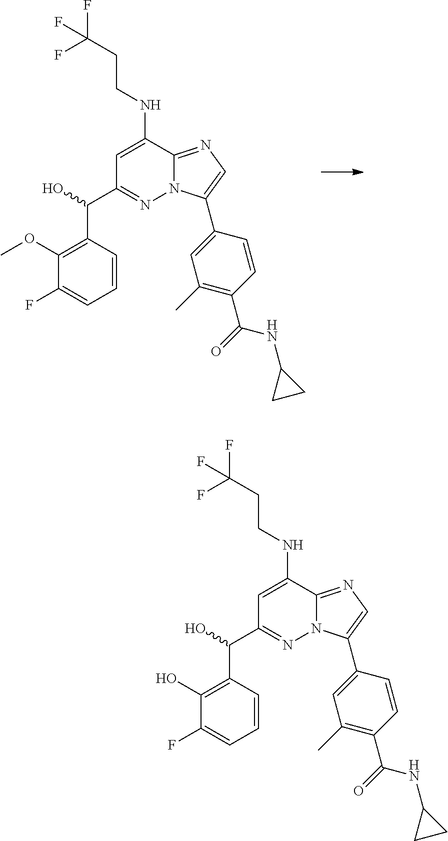 [SMILES: COc1c(F)cccc1C(O)c1cc(NCCC(F)(F)F)c2ncc(-c3ccc(C(=O)NC4CC4)c(C)c3)n2n1 ; Cc1cc(-c2cnc3c(NCCC(F)(F)F)cc(C(O)c4cccc(F)c4O)nn23)ccc1C(=O)NC1CC1]

A mixture of 25.0 mg (45 μmol) (RS)—N-cyclopropyl-4-{6-[(3-fluoro-2-methoxyphenyl)(hydroxy)methyl]-8-[(3,3,3-trifluoropropyl)amino]imidazo[1,2-b]pyridazin-3-yl}-2-methylbenzamide which was prepared according to intermediate example 3a, 25.1 mg sodium methanethiolate and 900 μL dimethyl sulfoxide was heated under microwave irradiation for 5 minutes at 130° C. Hydrochloric acid was added and the solvent removed. The residue was purified by chromatography to give 8.2 mg (32%) of the title compound.

¹H-NMR (DMSO-d₆): δ=0.50 (2H), 0.65 (2H), 2.29 (3H), 2.56-2.72 (2H), 2.79 (1H), 3.57 (2H), 6.00 (1H), 6.33 (1H), 6.33 (1H), 6.79 (1H), 7.03 (1H), 7.24 (1H), 7.27 (1H), 7.44 (1H), 7.86-7.92 (2H), 8.96 (1H), 8.24 (1H) ppm.

### Intermediate Example 3a

### (RS)—N-Cyclopropyl-4-{6-[(3-fluoro-2-methoxyphenyl)(hydroxy)methyl]-8-[(3,3,3-trifluoropropyl)amino]imidazo[1,2-b]pyridazin-3-yl}-2-methylbenzamide

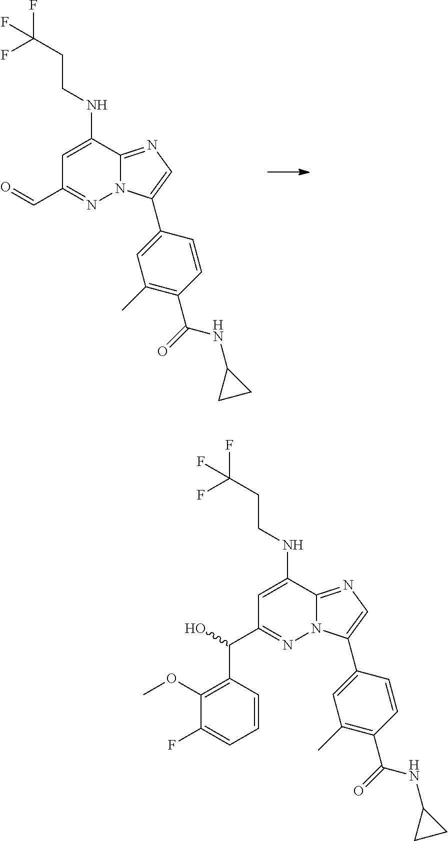

500 mg (1.16 mmol) N-cyclopropyl-4-{6-formyl-8-[(3,3,3-trifluoropropyl)amino]imidazo[1,2-b]pyridazin-3-yl}-2-methylbenzamide which was prepared according to intermediate example 1c were transformed in analogy to intermediate example 1b using bromo(3-fluoro-2-methoxyphenyl)magnesium to give after working up and purification 519 mg (80%) of the title compound.

### Compound A4

### (RS)—N-Cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide

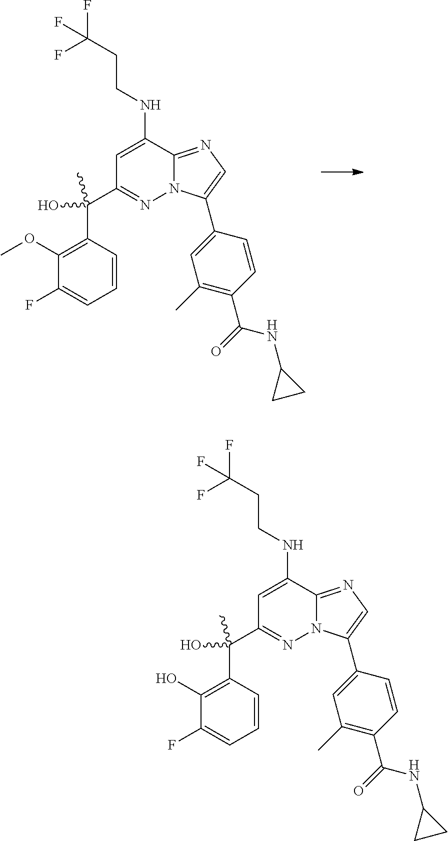

23.5 mg (41 μmol) (RS)—N-cyclopropyl-4-{6-[1-(3-fluoro-2-methoxyphenyl)-1-hydroxyethyl]-8-[(3,3,3-trifluoropropyl)amino]imidazo[1,2-b]pyridazin-3-yl}-2-methylbenzamide which was prepared according to intermediate example 4a were transformed in analogy to example 3 to give after working up and purification 9.5 mg (39%) of the title compound.

¹H-NMR (DMSO-d₆): δ=0.50 (2H), 0.65 (2H), 1.91 (3H), 2.32 (3H), 2.52-2.65 (2H), 2.80 (1H), 3.49 (2H), 6.22 (1H), 6.69 (1H), 7.00 (1H), 7.18 (1H), 7.28 (1H), 7.35 (1H), 7.93 (1H), 7.96-8.02 (2H), 8.25 (1H), 8.59 (1H) ppm.

### Intermediate Example 4a

### (RS)—N-Cyclopropyl-4-{6-[1-(3-fluoro-2-methoxyphenyl)-1-hydroxyethyl]-8-[(3,3,3-trifluoropropyl)amino]imidazo[1,2-b]pyridazin-3-yl}-2-methylbenzamide

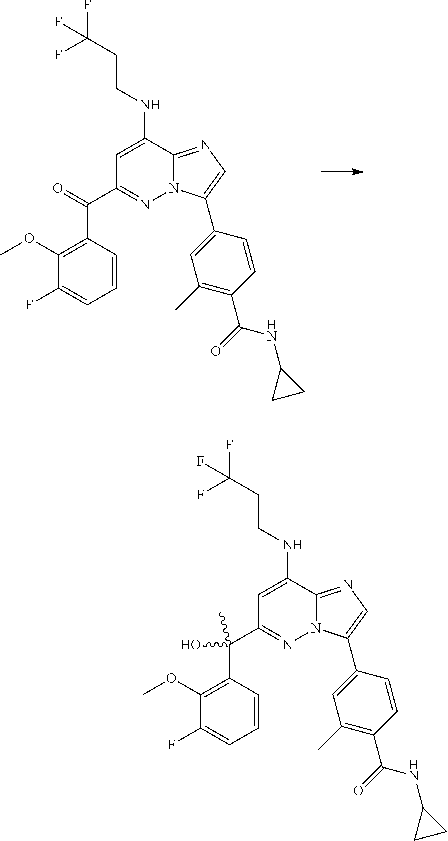

To a solution of 50 mg (90 μmol) N-cyclopropyl-4-{6-(3-fluoro-2-methoxybenzoyl)-8-[(3,3,3-trifluoropropyl)amino]imidazo[1,2-b]pyridazin-3-yl}-2-methylbenzamide which was prepared according to intermediate example 4b in 2.5 mL tetrahydrofuran at −78° C. were added 225 μL methyllithium (2.5M in diethyl ether). The mixture was stirred at −50° C. for 30 minutes, poured into water and extracted with ethyl acetate. The organic layer was washed with saturated aqueous ammonium chloride solution and dried over sodium sulfate. After filtration and removal of the solvent, the residue was purified by chromatography to give 29 mg (56%) of the title compound.

### Intermediate Example 4b

### N-Cyclopropyl-4-{6-(3-fluoro-2-methoxybenzoyl)-8-[(3,3,3-trifluoropropyl)amino]imidazo[1,2-b]pyridazin-3-yl}-2-methylbenzamide

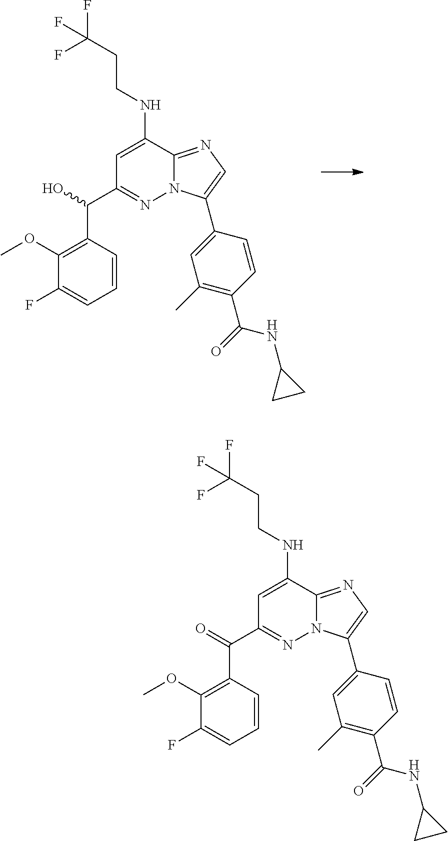 [SMILES: COc1c(F)cccc1C(O)c1cc(NCCC(F)(F)F)c2ncc(-c3ccc(C(=O)NC4CC4)c(C)c3)n2n1 ; COc1c(F)cccc1C(=O)c1cc(NCCC(F)(F)F)c2ncc(-c3ccc(C(=O)NC4CC4)c(C)c3)n2n1]

442 mg (793 μmol) (RS)—N-cyclopropyl-4-{6-[(3-fluoro-2-methoxyphenyl)(hydroxy) methyl]-8-[(3,3,3-trifluoropropyl)amino]imidazo[1,2-b]pyridazin-3-yl}-2-methylbenzamide which was prepared according to intermediate example 3a were transformed in analogy to intermediate example 1a to give after working up and purification 317 mg (72%) of the title compound.

### Compound A5

### (RS)—N-Cyclopropyl-4-{6-[fluoro(3-fluorophenyl)methyl]-8-[(3,3,3-trifluoropropyl)amino]imidazo[1,2-b]pyridazin-3-yl}-2-methylbenzamide

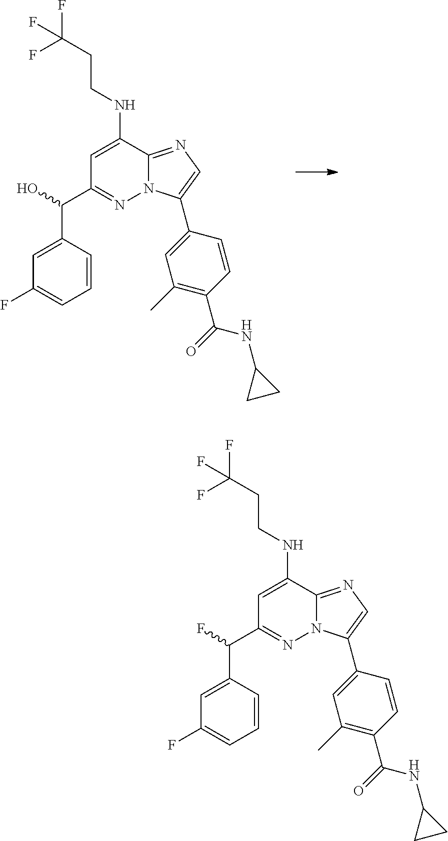 [SMILES: Cc1cc(-c2cnc3c(NCCC(F)(F)F)cc(C(O)c4cccc(F)c4)nn23)ccc1C(=O)NC1CC1 ; Cc1cc(-c2cnc3c(NCCC(F)(F)F)cc(C(F)c4cccc(F)c4)nn23)ccc1C(=O)NC1CC1]

50 mg (95 μmol) (RS)—N-cyclopropyl-4-{6-[(3-fluorophenyl)(hydroxy)methyl]-8-[(3,3,3-trifluoropropyl)amino]imidazo[1,2-b]pyridazin-3-yl}-2-methylbenzamide which was prepared according to intermediate example 5a were transformed in analogy to example 2 to give after working up and purification 4.3 mg (7%) of the title compound.

¹H-NMR (DMSO-d₆): δ=0.50 (2H), 0.65 (2H), 2.31 (3H), 2.50 (1H), 2.61-2.74 (2H), 2.80 (1H), 3.63 (2H), 6.41 (1H), 6.68 (1H), 7.22 (1H), 7.29 (1H), 7.33-7.39 (1H), 7.46 (1H), 7.75 (1H), 7.87-7.90 (2H), 8.02 (1H), 8.26 (1H) ppm.

### Intermediate Example 5a

### (RS)—N-Cyclopropyl-4-{6-[(3-fluorophenyl) (hydroxy)methyl]-8-[(3,3,3-trifluoropropyl)amino]imidazo[1,2-b]pyridazin-3-yl}-2-methylbenzamide

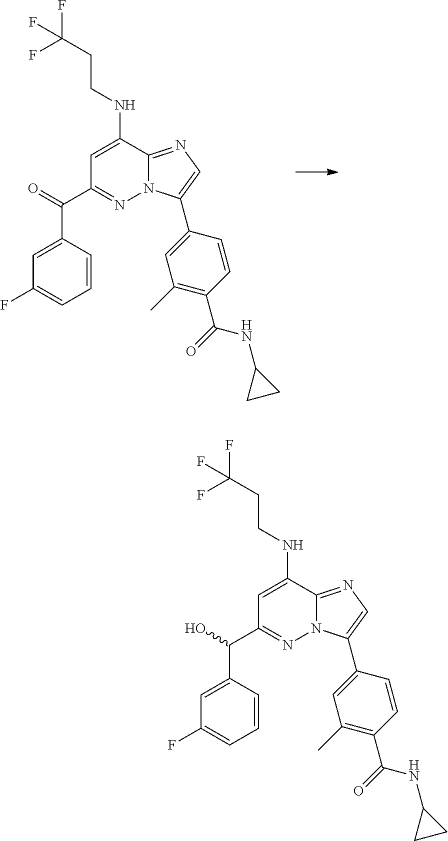 [SMILES: Cc1cc(-c2cnc3c(NCCC(F)(F)F)cc(C(=O)c4cccc(F)c4)nn23)ccc1C(=O)NC1CC1 ; Cc1cc(-c2cnc3c(NCCC(F)(F)F)cc(C(O)c4cccc(F)c4)nn23)ccc1C(=O)NC1CC1]

To a solution of 210 mg (400 μmol) N-cyclopropyl-4-{6-(3-fluorobenzoyl)-8-[(3,3,3-trifluoropropyl)amino]imidazo[1,2-b]pyridazin-3-yl}-2-methylbenzamide which was prepared according to comparative example 3b in 5 mL dichloromethane were added at 3° C. 151 mg sodium borohydride and stirring was continued for 1 hour and at 23° C. for 1 hour. Water was added and the organic layer was washed with water and dried over sodium sulfate. After filtration and removal of the solvent, 209 mg (96%) of the title compound were obtained that was used without further purification.

### Compound A6

### N-Cyclopropyl-4-{6-[difluoro(3-fluorophenyl)methyl]-8-[(3,3,3-trifluoropropyl)amino]imidazo[1, 2-b]pyridazin-3-yl}-2-methylbenzamide

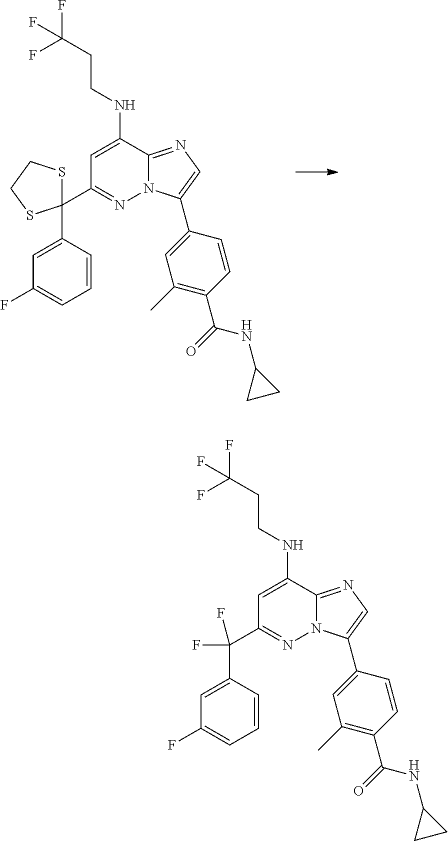 [SMILES: Cc1cc(-c2cnc3c(NCCC(F)(F)F)cc(C4(c5cccc(F)c5)SCCS4)nn23)ccc1C(=O)NC1CC1 ; Cc1cc(-c2cnc3c(NCCC(F)(F)F)cc(C(F)(F)c4cccc(F)c4)nn23)ccc1C(=O)NC1CC1]

28 mg (46 μmol) N-cyclopropyl-4-{6-[2-(3-fluorophenyl)-1,3-dithiolan-2-yl]-8-[(3,3,3-trifluoropropyl)amino]imidazo[1,2-b]pyridazin-3-yl}-2-methylbenzamide which was prepared according to comparative example 3a were transformed in analogy to the preparation described for compound A2 to give after working up and purification 7.9 mg (31%) of the title compound.

¹H-NMR (DMSO-d₆): δ=0.49 (2H), 0.65 (2H), 2.24 (3H), 2.61-2.84 (3H), 3.70 (2H), 6.61 (1H), 7.23 (1H), 7.41 (1H), 7.46-7.53 (2H), 7.57 (1H), 7.74 (1H), 7.75 (1H), 7.97 (1H), 8.09 (1H), 8.24 (1H) ppm.

### Compound A7

### N-Cyclopropyl-4-{6-(3-methoxybenzoyl)-8-[(3,3,3-trifluoropropyl)amino]imidazo[1,2-b]pyridazin-3-yl}-2-methylbenzamide

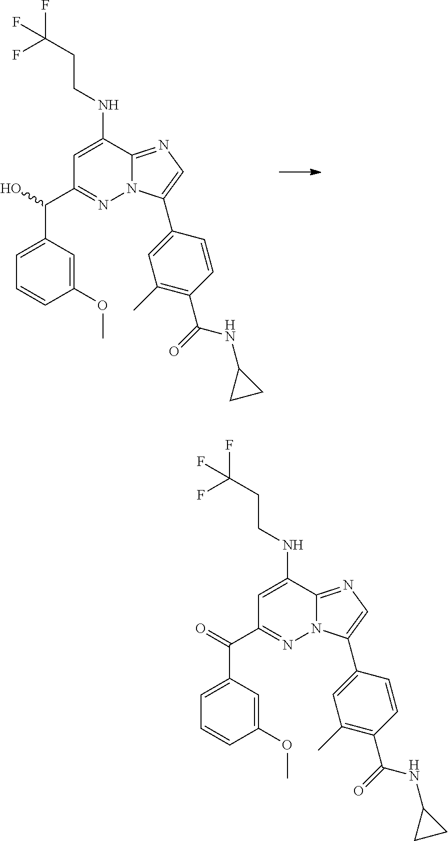

52 mg (96 μmol) (RS)—N-cyclopropyl-4-{6-[hydroxy(3-methoxyphenyl)methyl]-8-[(3,3,3-trifluoropropyl)amino]imidazo[1,2-b]pyridazin-3-yl}-2-methylbenzamide which was prepared according to intermediate example 7a were transformed in analogy to intermediate example 1a to give after working up and purification 34 mg (62%) of the title compound.

¹H-NMR (DMSO-d₆): δ=0.47 (2H), 0.64 (2H), 2.19 (3H), 2.62-2.82 (3H), 3.69 (2H), 3.76 (3H), 6.71 (1H), 7.23 (1H), 7.27 (1H), 7.48 (1H), 7.54 (1H), 7.61 (1H), 7.84 (1H), 7.89 (1H), 7.96 (1H), 8.15 (1H), 8.22 (1H) ppm.

### Intermediate Example 7a

### (RS)—N-cyclopropyl-4-{6-[hydroxy(3-methoxyphenyl)methyl]-8-[(3,3,3-trifluoropropyl)amino]imidazo[1,2-b]pyridazin-3-yl}-2-methylbenzamide

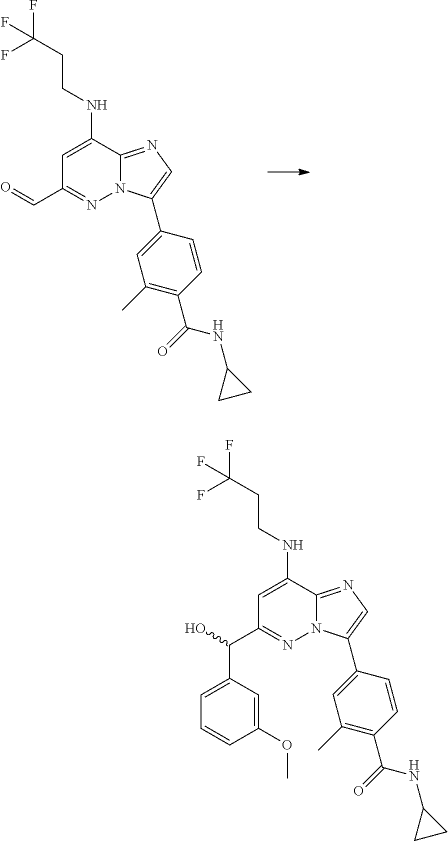

110 mg (255 μmol) N-cyclopropyl-4-{6-formyl-8-[(3,3,3-trifluoropropyl)amino]imidazo[1,2-b]pyridazin-3-yl}-2-methylbenzamide which was prepared according to intermediate example 1c were transformed in analogy to intermediate example 1b using bromo(3-methoxyphenyl)magnesium to give after working up and purification 79 mg (55%) of the title compound.

### Compound A8

### N-Cyclopropyl-4-{6-[1-(3-methoxyphenyl)vinyl]-8-[(3,3,3-trifluoropropyl)amino]imidazo[1, 2-b]pyridazin-3-yl}-2-methylbenzamide

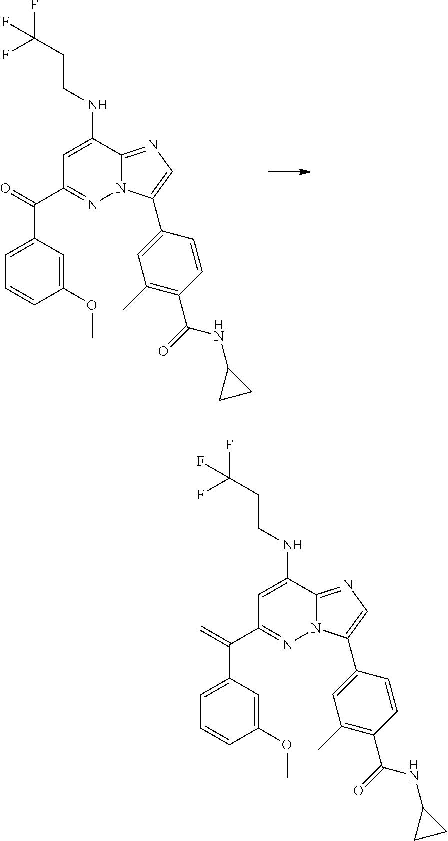

175 mg (326 μmol) N-cyclopropyl-4-{6-(3-methoxybenzoyl)-8-[(3,3,3-trifluoropropyl)amino]imidazo[1,2-b]pyridazin-3-yl}-2-methylbenzamide which was prepared according to the preparation method described for compound A7 were transformed in analogy to the preparation method described for compound A1 to give after working up and purification 96.3 mg (55%) of the title compound.

¹H-NMR (DMSO-d₆): δ=0.48 (2H), 0.64 (2H), 2.18 (3H), 2.60-2.72 (2H), 2.78 (1H), 3.60 (2H), 3.71 (3H), 5.75 (1H), 5.98 (1H), 6.34 (1H), 6.92-6.99 (3H), 7.21 (1H), 7.29 (1H), 7.53 (1H), 7.84 (1H), 7.89 (1H), 8.01 (1H), 8.21 (1H) ppm.

### Compound A9

### N-Cyclopropyl-4-{6-(4-methoxybenzoyl)-8-[(3,3,3-trifluoropropyl)amino]imidazo[1, 2-b]pyridazin-3-yl}-2-methylbenzamide

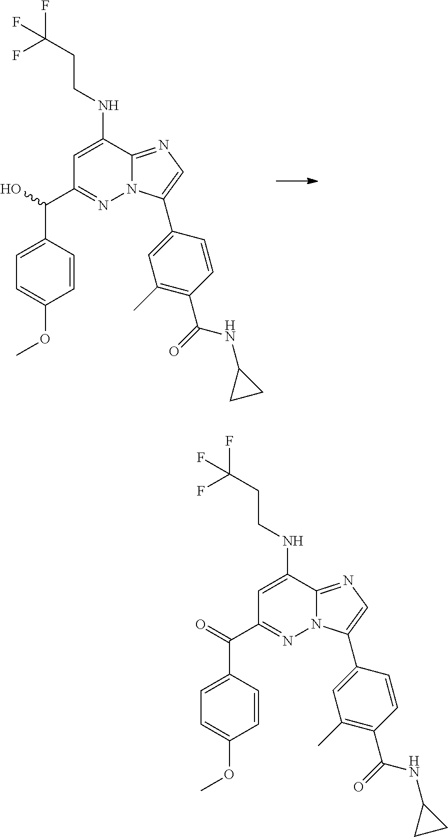

400 mg (741 μmol) (RS)—N-cyclopropyl-4-{6-[hydroxy(4-methoxyphenyl)methyl]-8-[(3,3,3-trifluoropropyl)amino]imidazo[1,2-b]pyridazin-3-yl}-2-methylbenzamide which was prepared according to intermediate example 9a were transformed in analogy to intermediate example 1a to give after working up and purification 264 mg (66%) of the title compound.

¹H-NMR (DMSO-d₆): δ=0.51 (2H), 0.66 (2H), 2.34 (3H), 2.56-2.72 (2H), 2.81 (1H), 3.56 (2H), 3.68 (3H), 6.31 (1H), 6.86 (2H), 7.33 (1H), 7.38 (2H), 7.49 (1H), 7.93-8.02 (3H), 8.29 (1H) ppm.

### Intermediate Example 9a

### (RS)—N-Cyclopropyl-4-{6-[hydroxy(4-methoxyphenyl)methyl]-8-[(3,3,3-trifluoropropyl)amino]imidazo[1,2-b]pyridazin-3-yl}-2-methylbenzamide

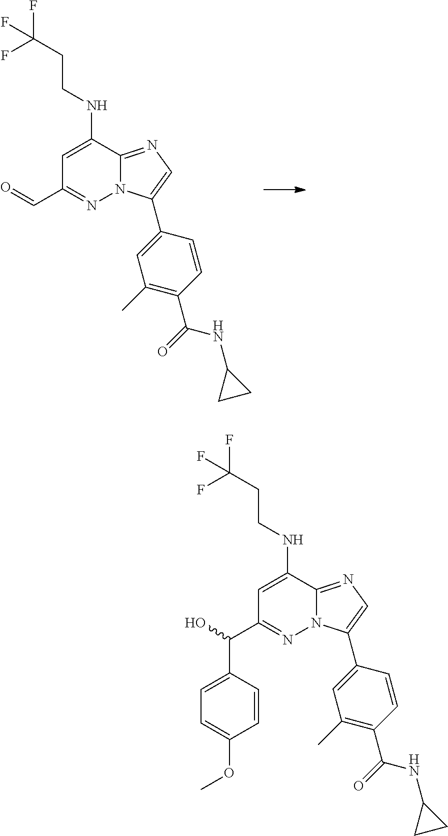

500 mg (1.16 mmol) N-cyclopropyl-4-{6-formyl-8-[(3,3,3-trifluoropropyl)amino]imidazo[1,2-b]pyridazin-3-yl}-2-methylbenzamide which was prepared according to intermediate example 1c were transformed in analogy to intermediate example 1b using bromo(4-methoxyphenyl)magnesium to give after working up and purification 501 mg (72%) of the title compound.

### Compound A10

### N-Cyclopropyl-4-{6-[1-(4-methoxyphenyl)vinyl]-8-[(3,3,3-trifluoropropyl)amino]imidazo[1,2-b]pyridazin-3-yl}-2-methylbenzamide

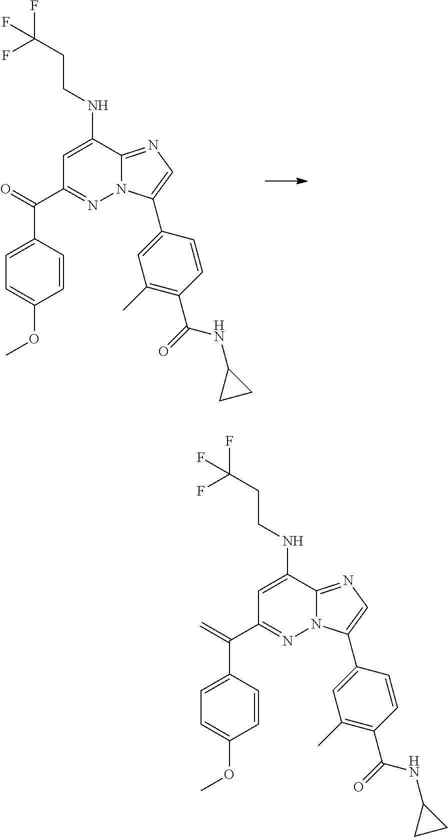

73 mg (136 μmol) N-cyclopropyl-4-{6-(4-methoxybenzoyl)-8-[(3,3,3-trifluoropropyl)amino]imidazo[1,2-b]pyridazin-3-yl}-2-methylbenzamide which was prepared according to the preparation method described for compound A9 were transformed in analogy to the preparation method described for compound A1 to give after working up and purification 36.1 mg (45%) of the title compound.

¹H-NMR (DMSO-d₆): δ=0.47 (2H), 0.63 (2H), 2.17 (3H), 2.60-2.72 (2H), 2.78 (1H), 3.60 (2H), 3.76 (3H), 5.66 (1H), 5.85 (1H), 6.32 (1H), 6.93 (2H), 7.22 (1H), 7.35 (2H), 7.52 (1H), 7.84 (1H), 7.89 (1H), 8.00 (1H), 8.21 (1H) ppm.

### Compound A11

### (RS)—N-Cyclopropyl-4-{6-[(2, 5-difluorophenyl)(hydroxy)methyl]-8-[(3,3,3-trifluoropropyl)amino]imidazo[1, 2-b]pyridazin-3-yl}-2-methylbenzamide

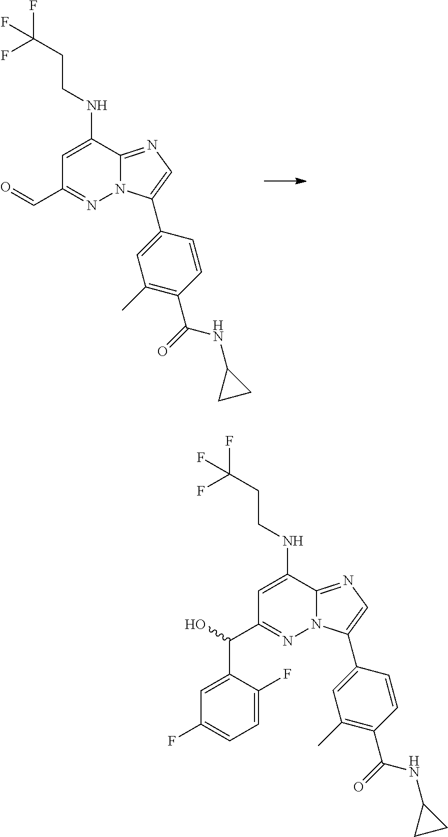

400 mg (927 μmol) N-cyclopropyl-4-{6-formyl-8-[(3,3,3-trifluoropropyl)amino]imidazo[1,2-b]pyridazin-3-yl}-2-methylbenzamide which was prepared according to intermediate example 1c were transformed in analogy to intermediate example 1b using bromo(2,5-difluorophenyl)magnesium to give after working up and purification 326 mg (64%) of the title compound.

¹H-NMR (DMSO-d₆): δ=0.49 (2H), 0.65 (2H), 2.27 (3H), 2.58-2.74 (2H), 2.79 (1H), 3.60 (2H), 5.94 (1H), 6.41 (1H), 6.54 (1H), 7.12-7.27 (3H), 7.44 (1H), 7.57 (1H), 7.82 (1H), 7.88 (1H), 7.99 (1H), 8.26 (1H) ppm.

### Compound A12

### N-Cyclopropyl-4-{6-(2, 5-difluorobenzoyl)-8-[(3,3,3-trifluoropropyl)amino]imidazo[1, 2-b]pyridazin-3-yl}-2-methylbenzamide

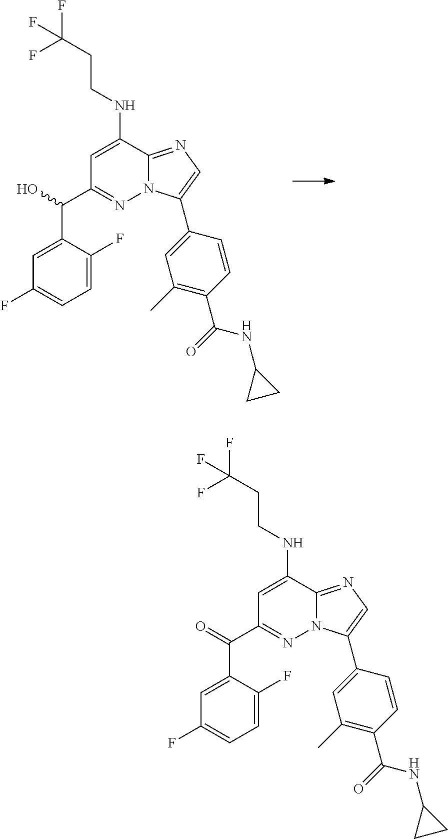

300 mg (550 μmol) (RS)—N-cyclopropyl-4-{6-[(2,5-difluorophenyl)(hydroxy)methyl]-8-[(3,3,3-trifluoropropyl)amino]imidazo[1,2-b]pyridazin-3-yl}-2-methylbenzamide which was prepared according to the method described for compound A11 were transformed in analogy to intermediate example 1a to give after working up and purification 82 mg (27%) of the title compound.

¹H-NMR (DMSO-d₆): δ=0.48 (2H), 0.64 (2H), 2.14 (3H), 2.63-2.82 (3H), 3.71 (2H), 6.79 (1H), 7.18 (1H), 7.46 (1H), 7.55 (1H), 7.65 (1H), 7.73 (1H), 7.81 (1H), 7.99 (1H), 8.18 (1H), 8.23 (1H) ppm.

### Compound A13

### N-Cyclopropyl-4-{6-(3-fluoro-4-methoxybenzoyl)-8-[(3,3,3-trifluoropropyl)amino]imidazo[1,2-b]pyridazin-3-yl}-2-methylbenzamide

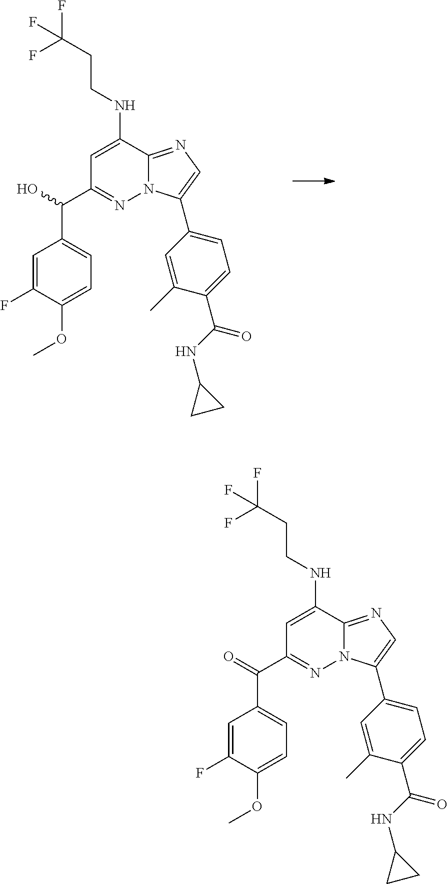 [SMILES: COc1ccc(C(O)c2cc(NCCC(F)(F)F)c3ncc(-c4ccc(C(=O)NC5CC5)c(C)c4)n3n2)cc1F ; COc1ccc(C(=O)c2cc(NCCC(F)(F)F)c3ncc(-c4ccc(C(=O)NC5CC5)c(C)c4)n3n2)cc1F]

262 mg (470 μmol) N-cyclopropyl-4-{6-[(3-fluoro-4-methoxyphenyl)(hydroxy)methyl]-8-[(3,3,3-trifluoropropyl)amino]imidazo[1,2-b]pyridazin-3-yl}-2-methylbenzamide which was prepared according to intermediate example 1b were transformed in analogy to intermediate example 1a to give after working up and purification 210 mg (80%) of the title compound.

¹H-NMR (DMSO-d₆): δ=0.48 (2H), 0.64 (2H), 2.24 (3H), 2.64-2.75 (2H), 2.78 (1H), 3.68 (2H), 3.94 (3H), 6.67 (1H), 7.28 (1H), 7.34 (1H), 7.85 (1H), 7.89 (1H), 7.93-7.99 (3H), 8.13 (1H), 8.24 (1H) ppm.

### Compound A14

### N-Cyclopropyl-4-{6-(2,3-difluorobenzoyl)-8-[(3,3,3-trifluoropropyl)amino]imidazo[1,2-b]pyridazin-3-yl}-2-methylbenzamide

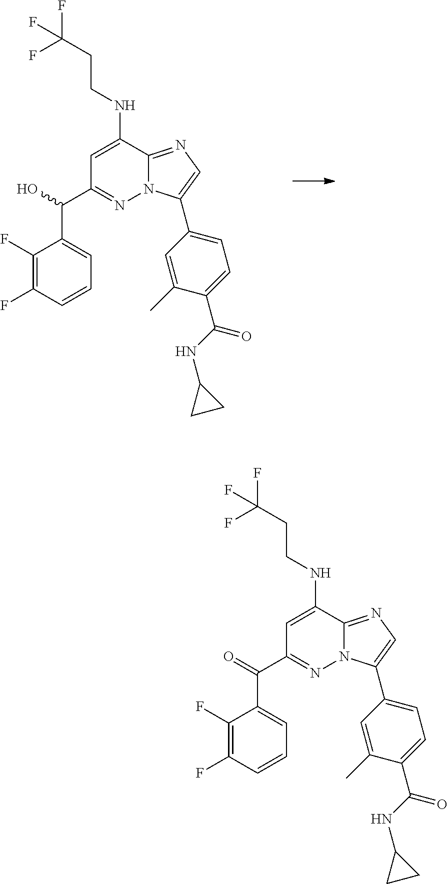 [SMILES: Cc1cc(-c2cnc3c(NCCC(F)(F)F)cc(C(O)c4cccc(F)c4F)nn23)ccc1C(=O)NC1CC1 ; Cc1cc(-c2cnc3c(NCCC(F)(F)F)cc(C(=O)c4cccc(F)c4F)nn23)ccc1C(=O)NC1CC1]

296 mg (543 μmol) N-cyclopropyl-4-{6-[(2,3-difluorophenyl)(hydroxy)methyl]-8-[(3,3,3-trifluoropropyl)amino]imidazo[1,2-b]pyridazin-3-yl}-2-methylbenzamide which was prepared according to intermediate example 14a were transformed in analogy to intermediate example 1a to give after working up and purification 165 mg (56%) of the title compound.

¹H-NMR (DMSO-d₆): δ=0.47 (2H), 0.64 (2H), 2.13 (3H), 2.63-2.82 (3H), 3.71 (2H), 6.80 (1H), 7.17 (1H), 7.40 (1H), 7.55 (1H), 7.68-7.84 (3H), 8.04 (1H), 8.19 (1H), 8.26 (1H) ppm.

### Intermediate Example 14a

### N-Cyclopropyl-4-{6-[(2,3-difluorophenyl) (hydroxy)methyl]-8-[(3,3,3-trifluoropropyl)amino]imidazo[1,2-b]pyridazin-3-yl}-2-methylbenzamide

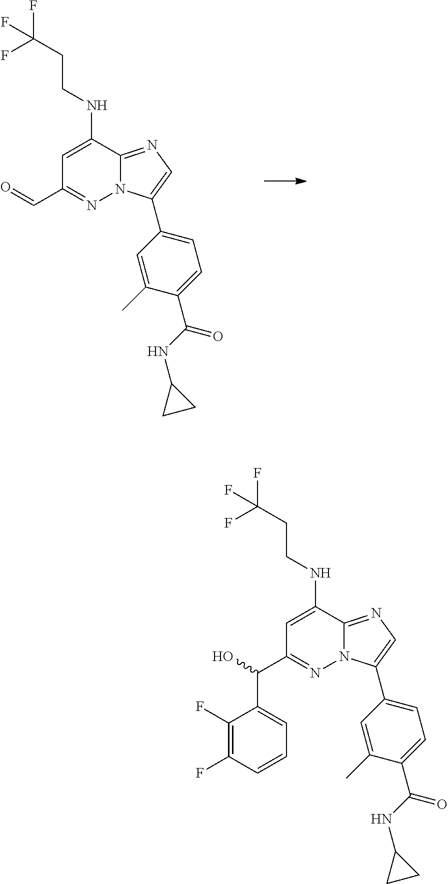 [SMILES: Cc1cc(-c2cnc3c(NCCC(F)(F)F)cc(C=O)nn23)ccc1C(=O)NC1CC1 ; Cc1cc(-c2cnc3c(NCCC(F)(F)F)cc(C(O)c4cccc(F)c4F)nn23)ccc1C(=O)NC1CC1]

400 mg (927 μmol) N-cyclopropyl-4-{6-formyl-8-[(3,3,3-trifluoropropyl)amino]imidazo[1,2-b]pyridazin-3-yl}-2-methylbenzamide which was prepared according to intermediate example 1c were transformed in analogy to intermediate example 1b using bromo(2,3-difluorophenyl)magnesium to give after working up and purification 326 mg (64%) of the title compound.

### Compound A15

### N-Cyclopropyl-4-{6-[1-(2,3-difluorophenyl)vinyl]-8-[(3, 3,3-trifluoropropyl)amino]imidazo[1, 2-b]pyridazin-3-yl}-2-methylbenzamide

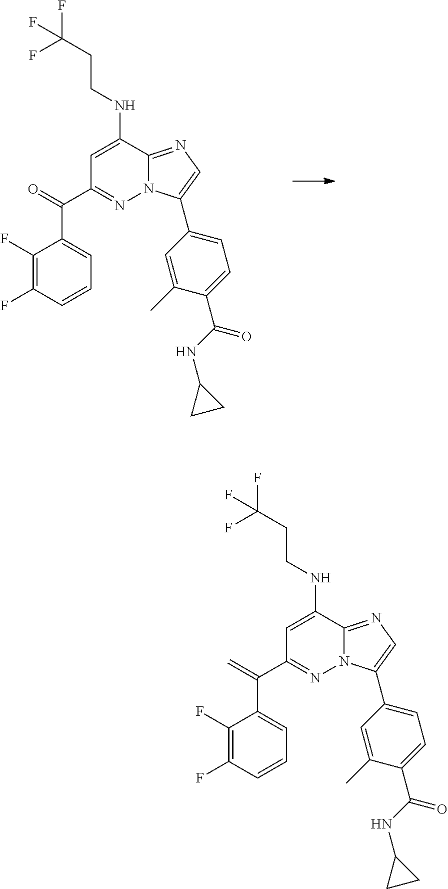 [SMILES: Cc1cc(-c2cnc3c(NCCC(F)(F)F)cc(C(=O)c4cccc(F)c4F)nn23)ccc1C(=O)NC1CC1 ; C=C(c1cc(NCCC(F)(F)F)c2ncc(-c3ccc(C(=O)NC4CC4)c(C)c3)n2n1)c1cccc(F)c1F]

75 mg (138 μmol) N-Cyclopropyl-4-{6-(2,3-difluorobenzoyl)-8-[(3,3,3-trifluoropropyl)amino]imidazo[1,2-b]pyridazin-3-yl}-2-methylbenzamide which was prepared according to the preparation method described for compound A14 were transformed in analogy to the preparation method described for compound A1 to give after working up and purification 67.2 mg (83%) of the title compound.

¹H-NMR (DMSO-d₆): δ=0.50 (2H), 0.66 (2H), 2.14 (3H), 2.68-2.83 (3H), 3.69 (2H), 5.81 (1H), 6.47 (1H), 6.65 (1H), 7.14 (1H), 7.22-7.32 (2H), 7.51 (1H), 7.58 (1H), 7.69 (1H), 7.75 (1H), 8.03 (1H), 8.24 (1H) ppm.

### Compound A16

### N-Cyclopropyl-4-{6-[difluoro(4-methoxyphenyl)methyl]-8-[(3,3,3-trifluoropropyl)amino]imidazo[1,2-b]pyridazin-3-yl}-2-methylbenzamide

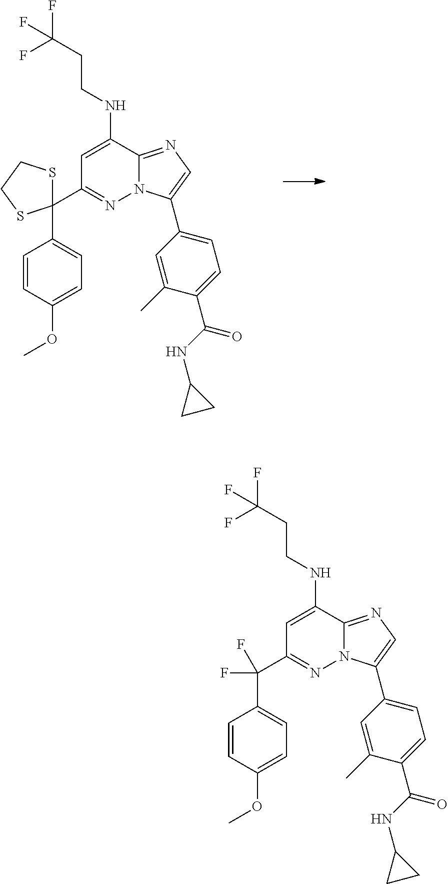 [SMILES: COc1ccc(C2(c3cc(NCCC(F)(F)F)c4ncc(-c5ccc(C(=O)NC6CC6)c(C)c5)n4n3)SCCS2)cc1 ; COc1ccc(C(F)(F)c2cc(NCCC(F)(F)F)c3ncc(-c4ccc(C(=O)NC5CC5)c(C)c4)n3n2)cc1]

30 mg (49 μmol) N-cyclopropyl-4-{6-[2-(4-methoxyphenyl)-1,3-dithiolan-2-yl]-8-[(3,3,3-trifluoropropyl)amino]imidazo[1,2-b]pyridazin-3-yl}-2-methylbenzamide which was prepared according to comparative example 5a were transformed in analogy to compound A2 to give after working up and purification 6.0 mg (21%) of the title compound.

¹H-NMR (DMSO-d₆): δ=0.49 (2H), 0.65 (2H), 2.23 (3H), 2.61-2.84 (3H), 3.67 (2H), 3.77 (3H), 6.55 (1H), 7.04 (2H), 7.24 (1H), 7.55 (2H), 7.78 (2H), 7.94 (1H), 8.09 (1H), 8.26 (1H) ppm.

### Compound A17

### N-Cyclopropyl-4-{6-[1-(2,3-difluorophenyl)cyclopropyl]-8-[(3, 3,3-trifluoropropyl)amino]imidazo[1, 2-b]pyridazin-3-yl}-2-methylbenzamide

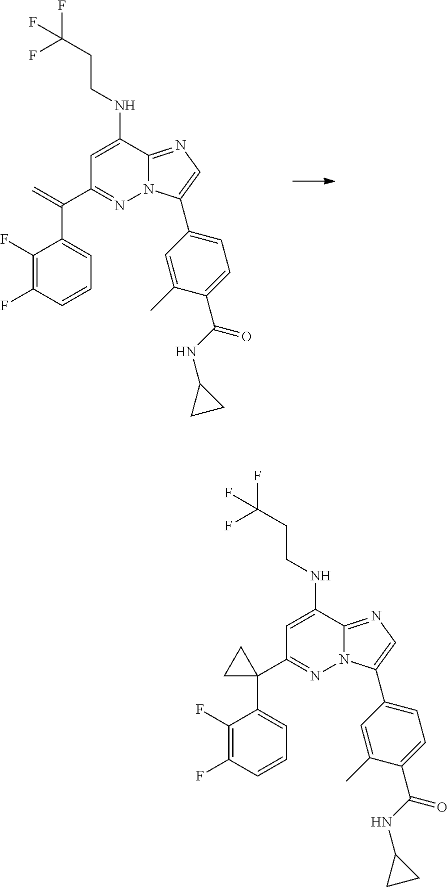 [SMILES: C=C(c1cc(NCCC(F)(F)F)c2ncc(-c3ccc(C(=O)NC4CC4)c(C)c3)n2n1)c1cccc(F)c1F ; Cc1cc(-c2cnc3c(NCCC(F)(F)F)cc(C4(c5cccc(F)c5F)CC4)nn23)ccc1C(=O)NC1CC1]

A mixture comprising 68.3 mg [iodo(dimethyl)oxido-lambda⁶-sulfanyl]methane, 12.3 mg sodium hydride (60%) and 0.82 mL dimethyl sulfoxide was stirred at 60° C. for 1.5 hours. A solution of 21 mg (39 μmol) N-cyclopropyl-4-{6-[1-(2,3-difluorophenyl)vinyl]-8-[(3,3,3-trifluoropropyl)amino]imidazo[1,2-b]pyridazin-3-yl}-2-methylbenzamide which was prepared according to example 15 in 0.43 mL dimethyl sulfoxide was added and stirring continued at 130° C. under microwave irradiation for 1.5 hours. Water was added and the mixture extracted with ethyl acetate. The organic layer was washed with brine and dried over sodium sulfate. After filtration and removal of the solvent the residue was purified by chromatography to give 9.8 mg (41%) of the title compound.

¹H-NMR (DMSO-d₆): δ=0.49 (2H), 0.65 (2H), 1.36 (2H), 1.65 (2H), 2.24 (3H), 2.50-2.67 (2H), 2.80 (1H), 3.52 (2H), 5.73 (1H), 7.16 (1H), 7.22 (1H), 7.30 (1H), 7.34-7.49 (2H), 7.74 (1H), 7.79 (1H), 7.96 (1H), 8.26 (1H) ppm.

### Compound A18

### N-Cyclopropyl-4-{6-[(2,3-difluorophenyl)(difluoro)methyl]-8-[(3,3,3-trifluoropropyl)amino]imidazo[1,2-b]pyridazin-3-yl}-2-methylbenzamide

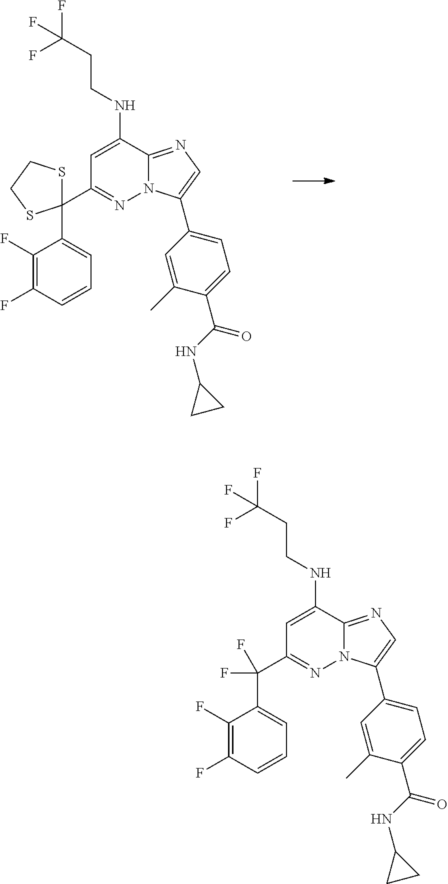 [SMILES: Cc1cc(-c2cnc3c(NCCC(F)(F)F)cc(C4(c5cccc(F)c5F)SCCS4)nn23)ccc1C(=O)NC1CC1 ; Cc1cc(-c2cnc3c(NCCC(F)(F)F)cc(C(F)(F)c4cccc(F)c4F)nn23)ccc1C(=O)NC1CC1]

21 mg (34 μmol) N-cyclopropyl-4-{6-[2-(2,3-difluorophenyl)-1,3-dithiolan-2-yl]-8-[(3,3,3-trifluoropropyl)amino]imidazo[1,2-b]pyridazin-3-yl}-2-methylbenzamide which was prepared according to intermediate example 18a were transformed in analogy to compound A2 to give after working up and purification 7.8 mg (41%) of the title compound.

¹H-NMR (DMSO-d₆): δ=0.48 (2H), 0.64 (2H), 2.15 (3H), 2.62-2.82 (3H), 3.71 (2H), 6.66 (1H), 7.14 (1H), 7.43 (1H), 7.56 (1H), 7.61 (1H), 7.65 (1H), 7.75 (1H), 8.07 (1H), 8.11 (1H), 8.26 (1H) ppm.

### Intermediate Example 18a

### N-Cyclopropyl-4-{6-[2-(2,3-difluorophenyl)-1,3-dithiolan-2-yl]-8-[(3,3,3-trifluoropropyl)amino]imidazo[1,2-b]pyridazin-3-yl}-2-methylbenzamide

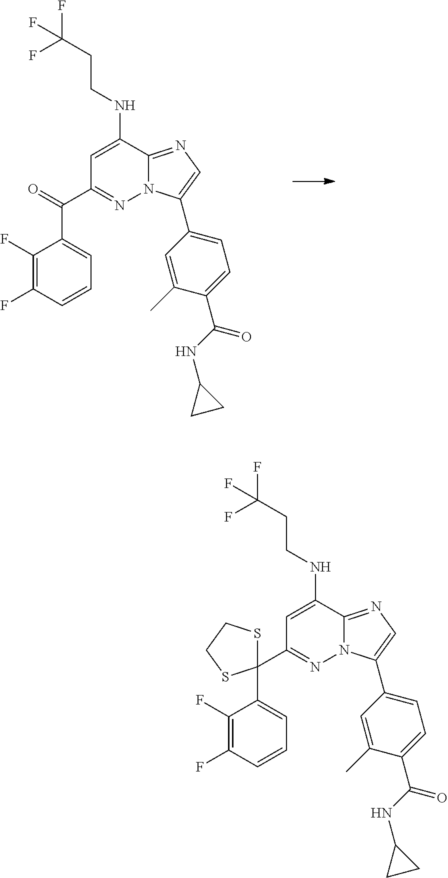 [SMILES: Cc1cc(-c2cnc3c(NCCC(F)(F)F)cc(C(=O)c4cccc(F)c4F)nn23)ccc1C(=O)NC1CC1 ; Cc1cc(-c2cnc3c(NCCC(F)(F)F)cc(C4(c5cccc(F)c5F)SCCS4)nn23)ccc1C(=O)NC1CC1]

50 mg (92 μmol) N-cyclopropyl-4-{6-(2,3-difluorobenzoyl)-8-[(3,3,3-trifluoropropyl)amino]imidazo[1,2-b]pyridazin-3-yl}-2-methylbenzamide which was prepared according to the preparation method described for compound A14 were transformed in analogy to comparative example 2b to give after working up and purification 23.6 mg (41%) of the title compound.

### Compound A19

### N-Cyclopropyl-4-{6-[1-(2, 5-difluorophenyl)vinyl]-8-[(3,3,3-trifluoropropyl)amino]imidazo[1,2-b]pyridazin-3-yl}-2-methylbenzamide

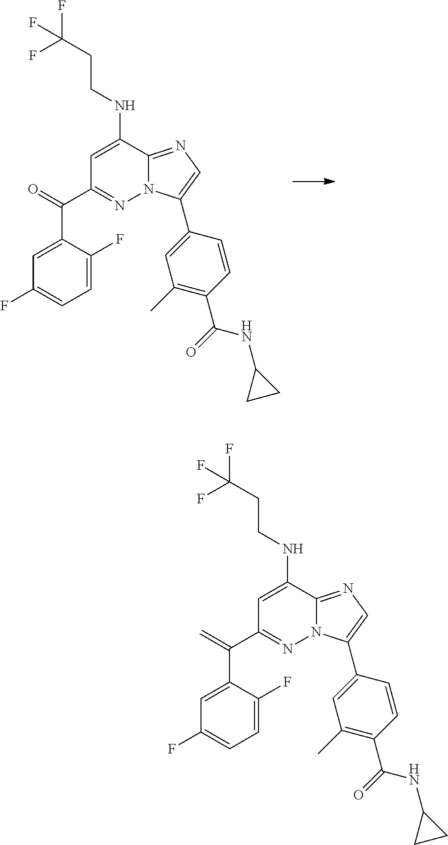 [SMILES: Cc1cc(-c2cnc3c(NCCC(F)(F)F)cc(C(=O)c4cc(F)ccc4F)nn23)ccc1C(=O)NC1CC1 ; C=C(c1cc(NCCC(F)(F)F)c2ncc(-c3ccc(C(=O)NC4CC4)c(C)c3)n2n1)c1cc(F)ccc1F]

150 mg (276 μmol) N-cyclopropyl-4-{6-(2,5-difluorobenzoyl)-8-[(3,3,3-trifluoropropyl)amino]imidazo[1,2-b]pyridazin-3-yl}-2-methylbenzamide which was prepared according to the preparation method described for compound A12 were transformed in analogy to compound A1 to give after working up and purification 96.2 mg (61%) of the title compound.

¹H-NMR (DMSO-d₆): δ=0.47 (2H), 0.64 (2H), 2.12 (3H), 2.62-2.81 (3H), 3.66 (2H), 5.78 (1H), 6.42 (1H), 6.61 (1H), 7.12 (1H), 7.23-7.35 (3H), 7.56 (1H), 7.67 (1H), 7.75 (1H), 8.01 (1H), 8.23 (1H) ppm.

### Compound A20

### N-Cyclopropyl-4-{6-[1-(2, 5-difluorophenyl)cyclopropyl]-8-[(3, 3,3-trifluoropropyl)amino]imidazo[1, 2-b]pyridazin-3-yl}-2-methylbenzamide

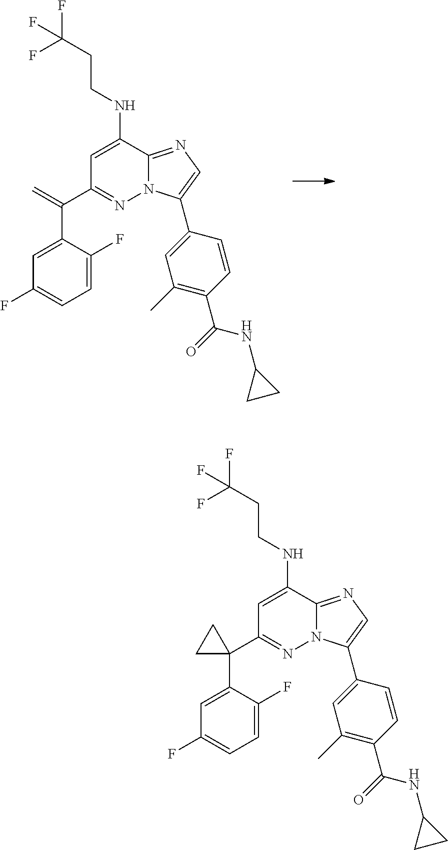 [SMILES: C=C(c1cc(NCCC(F)(F)F)c2ncc(-c3ccc(C(=O)NC4CC4)c(C)c3)n2n1)c1cc(F)ccc1F ; Cc1cc(-c2cnc3c(NCCC(F)(F)F)cc(C4(c5cc(F)ccc5F)CC4)nn23)ccc1C(=O)NC1CC1]

30 mg (55 μmol) N-cyclopropyl-4-{6-[1-(2,5-difluorophenyl)vinyl]-8-[(3,3,3-trifluoropropyl)amino]imidazo[1,2-b]pyridazin-3-yl}-2-methylbenzamide which was prepared according to the preparation method described for compound A19 were transformed in analogy to compound A17 to give after working up and purification 9.9 mg (31%) of the title compound.

¹H-NMR (DMSO-d₆): δ=0.50 (2H), 0.65 (2H), 1.36 (2H), 1.63 (2H), 2.26 (3H), 2.52-2.64 (2H), 2.80 (1H), 3.52 (2H), 5.75 (1H), 7.16-7.26 (3H), 7.35 (1H), 7.40 (1H) 7.75 (1H), 7.81 (1H), 7.95 (1H), 8.23 (1H) ppm.

### Compound A21

### N-Cyclopropyl-4-{6-[(2, 5-difluorophenyl)(difluoro)methyl]-8-[(3,3,3-trifluoropropyl)amino]imidazo[1, 2-b]pyridazin-3-yl}-2-methylbenzamide

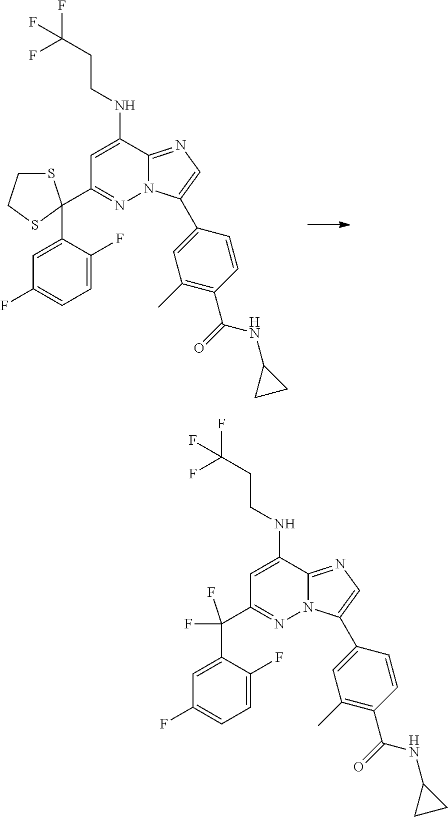

48 mg (77 μmol) N-cyclopropyl-4-{6-[2-(2,5-difluorophenyl)-1,3-dithiolan-2-yl]-8-[(3,3,3-trifluoropropyl)amino]imidazo[1,2-b]pyridazin-3-yl}-2-methylbenzamide which was prepared according to intermediate example 21a were transformed in analogy to comparative example 2b to give after working up and purification 17.3 mg (38%) of the title compound.

¹H-NMR (DMSO-d₆): δ=0.48 (2H), 0.65 (2H), 2.16 (3H), 2.64-2.82 (3H), 3.71 (2H), 6.65 (1H), 7.16 (1H), 7.46 (1H), 7.56 (1H), 7.60-7.65 (2H), 7.67 (1H), 8.03 (1H), 8.11 (1H), 8.23 (1H) ppm.

### Intermediate Example 21a

### N-Cyclopropyl-4-{6-[2-(2, 5-difluorophenyl)-1,3-dithiolan-2-yl]-8-[(3,3,3-trifluoropropyl)amino]imidazo[1,2-b]pyridazin-3-yl}-2-methylbenzamide

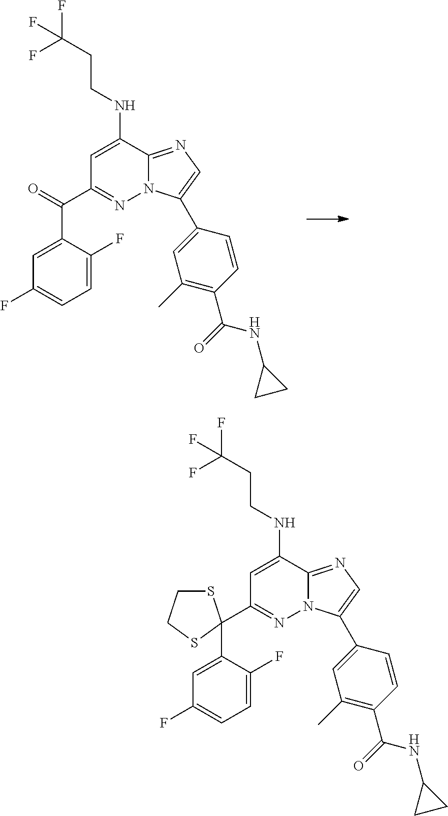

100 mg (184 μmol) N-cyclopropyl-4-{6-(2,5-difluorobenzoyl)-8-[(3,3,3-trifluoropropyl)amino]imidazo[1,2-b]pyridazin-3-yl}-2-methylbenzamide which was prepared according to the preparation method described for compound A12 were transformed in analogy to comparative example 2b to give after working up and purification 54 mg (47%) of the title compound.

### Compound A22

### N-cyclopropyl-4-{6-[1-(5-fluoro-2-hydroxyphenyl)ethenyl]-8-[(3,3,3-trifluoropropyl)amino]imidazo[1, 2-b]pyridazin-3-yl}-2-methylbenzamide

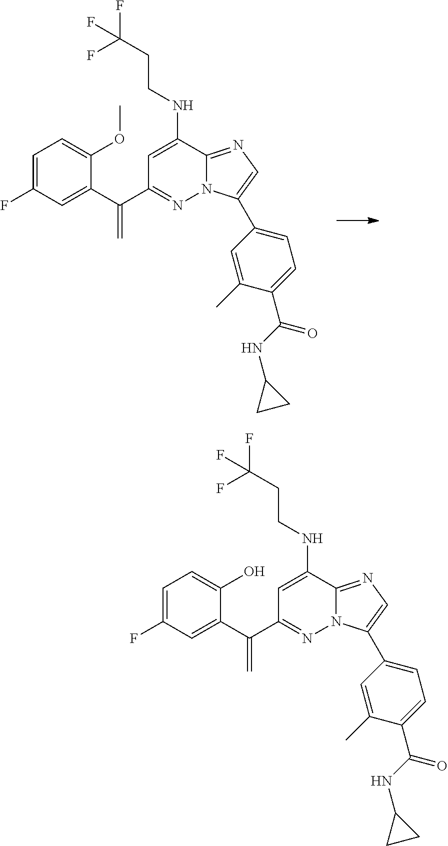

A mixture of 27.0 mg (49 μmol) N-cyclopropyl-4-{6-[1-(5-fluoro-2-methoxyphenyl)ethenyl]-8-[(3,3,3-trifluoropropyl)amino]imidazo[1,2-b]pyridazin-3-yl}-2-methylbenzamide which was prepared according to intermediate example 22a, 85.5 mg tribromoborane and 2000 μL DCM was stirred under ice cooling for 30 min and gave, after working-up and purification, 5.7 mg (22%) of the title compound.

¹H-NMR (DMSO-d₆): δ=0.44-0.52 (2H), 0.64 (2H), 2.15 (3H), 2.59-2.73 (2H), 2.78 (1H), 3.61 (2H), 5.59 (1H), 6.17 (1H), 6.41 (1H), 6.79 (1H), 7.00 (2H), 7.15 (1H), 7.41 (1H), 7.71-7.78 (1H), 7.84 (1H), 7.98 (1H), 8.20 (1H), 9.21 (1H) ppm.

### Intermediate Example 22a

### N-cyclopropyl-4-{6-[1-(5-fluoro-2-methoxyphenyl)ethenyl]-8-[(3,3,3-trifluoropropyl)amino]imidazo[1,2-b]pyridazin-3-yl}-2-methylbenzamide

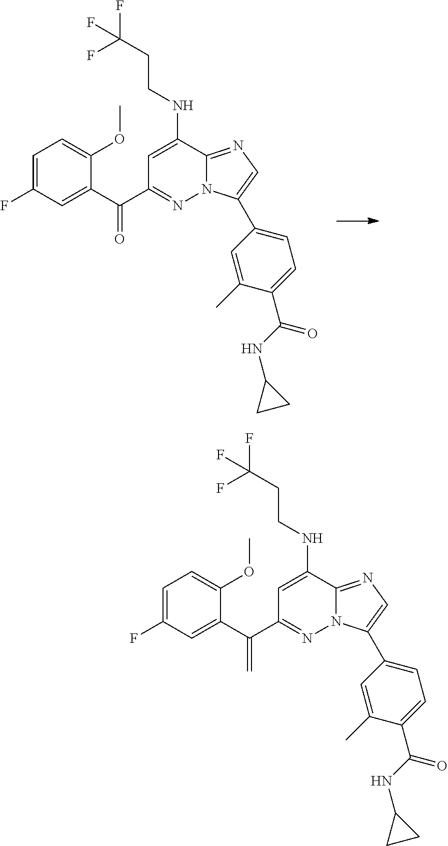

1740 mg (1.16 mmol) N-cyclopropyl-4-{6-(5-fluoro-2-methoxybenzoyl)-8-[(3,3,3-trifluoropropyl)amino]imidazo[1,2-b]pyridazin-3-yl}-2-methylbenzamide which was prepared according to intermediate example 22b were transformed in analogy to compound A1 to give after working up 1270 mg (73%) of the title compound.

¹H-NMR (DMSO-d₆): δ=0.43-0.52 (2H), 0.59-0.69 (2H), 2.14 (3H), 2.57-2.73 (2H), 2.73-2.84 (1H), 3.50 (3H), 3.56-3.67 (2H), 5.59 (1H), 6.25 (1H), 6.45 (1H), 6.98-7.16 (3H), 7.19 (1H), 7.46 (1H), 7.71 (1H), 7.78 (1H), 8.00 (1H), 8.23 (1H) ppm.

### Intermediate Example 22b

### N-cyclopropyl-4-{6-(5-fluoro-2-methoxybenzoyl)-8-[(3,3,3-trifluoropropyl)amino]imidazo[1,2-b]pyridazin-3-yl}-2-methylbenzamide

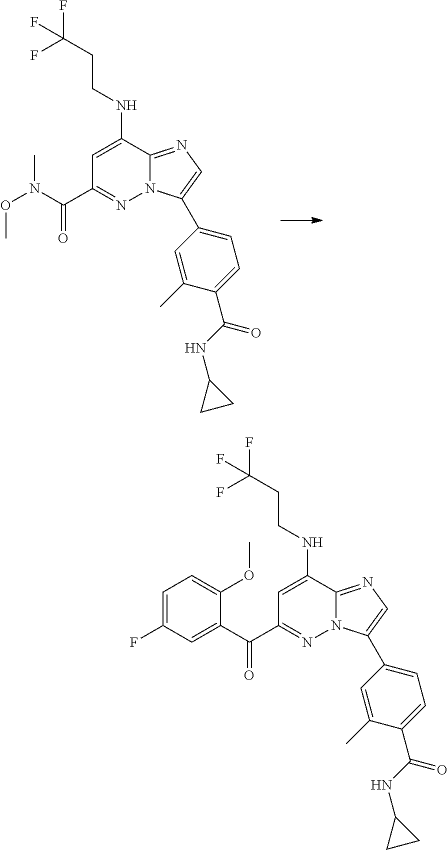

2000 mg (4.078 mmol) 3-[4-(cyclopropylcarbamoyl)-3-methylphenyl]-N-methoxy-N-methyl-8-[(3,3,3-trifluoropropyl)amino]imidazo[1,2-b]pyridazine-6-carboxamide which was prepared according to comparative example 3c were transformed in analogy to comparative example 3b using bromo(5-fluoro-2-methoxyphenyl)magnesium to give after working up 1740 mg (77%) of the title compound.

¹H-NMR (DMSO-d₆): δ=0.43-0.51 (2H), 0.60-0.69 (2H), 2.13 (3H), 2.62-2.83 (4H), 3.60 (3H), 3.70 (2H), 6.75 (1H), 7.15 (1H), 7.21 (1H), 7.33-7.46 (2H), 7.72 (1H), 7.80 (1H), 7.92 (1H), 8.18 (1H), 8.25 (1H) ppm.

### Compound A23

### N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide

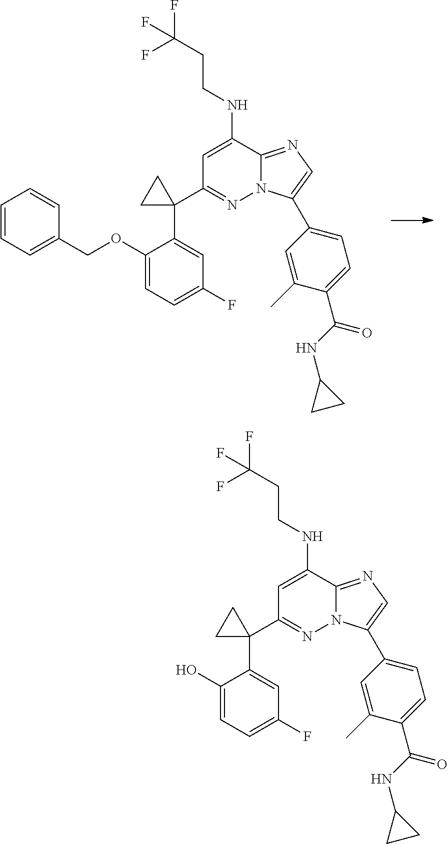 [SMILES: Cc1cc(-c2cnc3c(NCCC(F)(F)F)cc(C4(c5cc(F)ccc5OCc5ccccc5)CC4)nn23)ccc1C(=O)NC1CC1 ; Cc1cc(-c2cnc3c(NCCC(F)(F)F)cc(C4(c5cc(F)ccc5O)CC4)nn23)ccc1C(=O)NC1CC1]

A mixture of 584 mg (907 μmol) 4-(6-{1-[2-(benzyloxy)-5-fluorophenyl]cyclopropyl}-8-[(3,3,3-trifluoropropyl)amino]imidazo[1,2-b]pyridazin-3-yl)-N-cyclopropyl-2-methylbenzamide which was prepared according to intermediate example 23a and 50 mg Pd/C in 50 mL ethanol 7HOAC 8:2 was stirred at rt under a hydrogen atmosphere at 1 atm for 8 days and gave, after working-up 68 mg (14%) of the title compound.

¹H-NMR (DMSO-d₆): δ=0.47-0.58 (2H), 0.63-0.72 (2H), 1.21-1.29 (2H), 1.55-1.63 (2H), 2.51-2.65 (3H), 2.82 (1H), 3.46 (2H), 5.75 (1H), 6.81 (1H), 6.93-7.03 (1H), 7.11 (1H), 7.27 (1H), 7.37 (1H), 7.85-8.01 (3H), 8.30 (1H), 9.33 (1H) ppm.

### Intermediate Example 23a

### 4-(6-{1-[2-(benzyloxy)-5-fluorophenyl]cyclopropyl}-8-[(3,3,3-trifluoropropyl)amino]imidazo[1,2-b]pyridazin-3-yl)-N-cyclopropyl-2-methylbenzamide

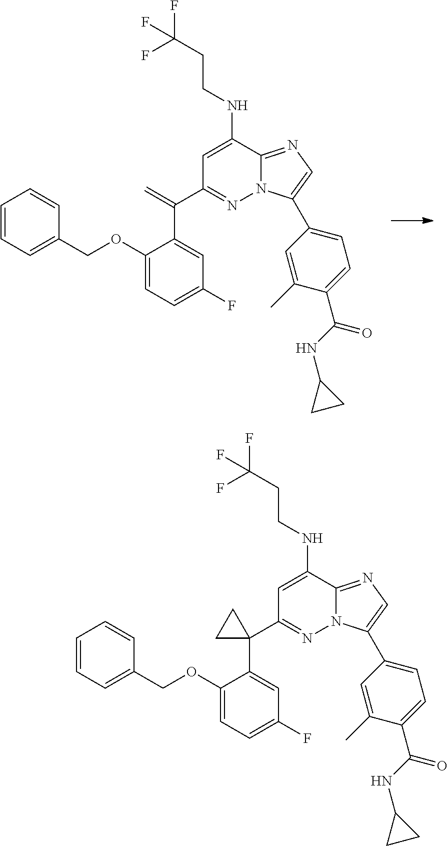 [SMILES: C=C(c1cc(NCCC(F)(F)F)c2ncc(-c3ccc(C(=O)NC4CC4)c(C)c3)n2n1)c1cc(F)ccc1OCc1ccccc1 ; Cc1cc(-c2cnc3c(NCCC(F)(F)F)cc(C4(c5cc(F)ccc5OCc5ccccc5)CC4)nn23)ccc1C(=O)NC1CC1]

2060 mg crude (3.27 mmol) 4-(6-{1-[2-(benzyloxy)-5-fluorophenyl]ethenyl}-8-[(3,3,3-trifluoropropyl)amino]imidazo[1,2-b]pyridazin-3-yl)-N-cyclopropyl-2-methylbenzamide which was prepared according to intermediate example 23b were transformed in analogy to compound A17 to give after working up and purification 692 mg (33%) of the title compound.

¹H-NMR (DMSO-d₆): δ=0.49-0.57 (2H), 0.64-0.73 (2H), 1.26-1.33 (2H), 1.58-1.66 (2H), 2.31 (3H), 2.42-2.55 (2H), 2.83 (1H), 3.41 (2H), 4.98 (2H), 5.72 (1H), 6.97-7.04 (2H), 7.05-7.11 (4H), 7.14 (1H), 7.25-7.30 (2H), 7.32 (1H), 7.83-7.88 (1H), 7.92 (1H), 7.96 (1H), 8.26 (1H) ppm.

### Intermediate Example 23b

### 4-(6-{1-[2-(benzyloxy)-5-fluorophenyl]but-3-en-1-yl}-8-[(3,3,3-trifluoropropyl)amino]imidazo[1,2-b]pyridazin-3-yl)-N-cyclopropyl-2-methylbenzamide

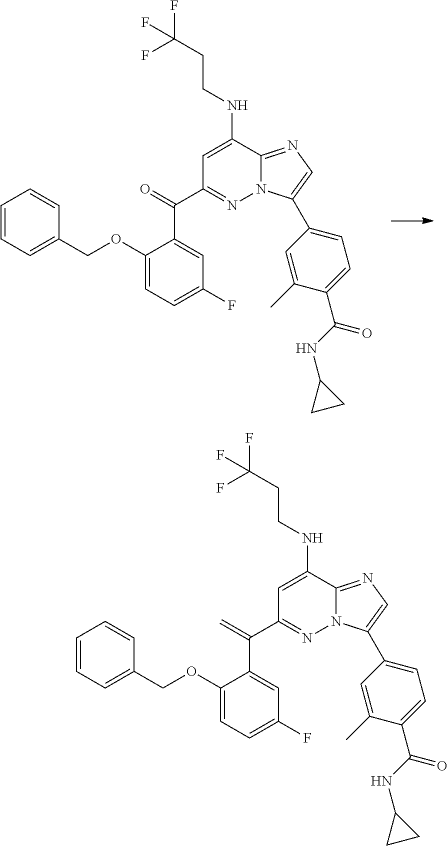 [SMILES: Cc1cc(-c2cnc3c(NCCC(F)(F)F)cc(C(=O)c4cc(F)ccc4OCc4ccccc4)nn23)ccc1C(=O)NC1CC1 ; C=C(c1cc(NCCC(F)(F)F)c2ncc(-c3ccc(C(=O)NC4CC4)c(C)c3)n2n1)c1cc(F)ccc1OCc1ccccc1]

14.06 g (22.26 mmol) 4-{6-[2-(benzyloxy)-5-fluorobenzoyl]-8-[(3,3,3-trifluoropropyl)amino]imidazo[1,2-b]pyridazin-3-yl}-N-cyclopropyl-2-methylbenzamide which was prepared according to intermediate example 23c were transformed in analogy to compound A1 to give after working up 21.83 g (150%) of the crude title compound which was used without further purification in the next step.

¹H-NMR (DMSO-d₆): δ=0.46-0.54 (2H), 0.62-0.70 (2H), 2.14 (3H), 2.52-2.68 (2H), 2.74-2.87 (1H), 3.59 (2H), 4.84 (2H), 5.65 (1H), 6.16 (1H), 6.39 (1H), 6.77 (2H), 6.93 (2H), 7.02-7.29 (5H), 7.43 (1H), 7.74 (1H), 7.82 (1H), 8.03 (1H), 8.21 (1H) ppm.

### Intermediate Example 23c

### 4-{6-[2-(benzyloxy)-5-fluorobenzoyl]-8-[(3,3,3-trifluoropropyl)amino]imidazo[1,2-b]pyridazin-3-yl}-N-cyclopropyl-2-methylbenzamide

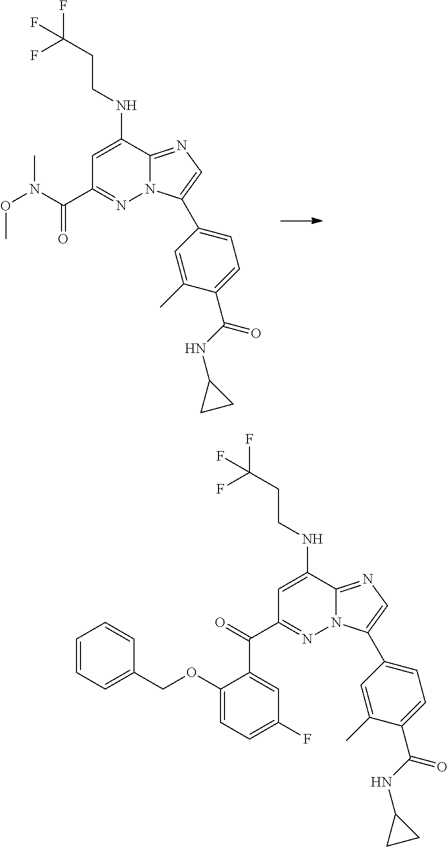

15.96 g (32.54 mmol) 3-[4-(cyclopropylcarbamoyl)-3-methylphenyl]-N-methoxy-N-methyl-8-[(3,3,3-trifluoropropyl)amino]imidazo[1,2-b]pyridazine-6-carboxamide which was prepared according to comparative example 3c in 300 mL THF were transformed in analogy to comparative example 3b using a freshly prepared solution of [2-(benzyloxy)-5-fluorophenyl](bromo)magnesium (231 mmol in 200 mL THF) to give after working up 13.26 g (64.5%) of the title compound.

¹H-NMR (DMSO-d₆): δ=0.47-0.56 (2H), 0.62-0.71 (2H), 2.12 (3H), 2.67 (2H), 2.80 (1H), 3.68 (2H), 4.96 (2H), 6.68 (1H), 6.83 (2H), 6.97 (2H), 7.07 (1H), 7.18 (1H), 7.32 (1H), 7.40-7.50 (2H), 7.73 (1H), 7.81 (1H), 7.88 (1H), 8.20 (1H), 8.24 (1H) ppm.

### Intermediate Example 23d

### [2-(benzyloxy)-5-fluorophenyl](bromo)magnesium

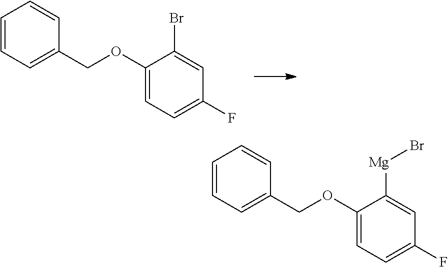

To stirred suspension of 5.62 g (231 mmol) magnesium in 100 mL THF were added at rt under an argon atmosphere one crystal of iodine and dropwise 40 mL of a solution of 64.95 g (231 mmol) 1-(benzyloxy)-2-bromo-4-fluorobenzene in 100 mL THF. The mixture was heated to 60° C. until decolorization and the remaining solution of 1-(benzyloxy)-2-bromo-4-fluorobenzene was added dropwise while keeping the temperature at 50° C.

After cooling to rt, the Grignard solution was directly used for intermediate example 23c.

### Compound A24

### N-cyclopropyl-4-{6-[1-(3-fluorophenyl)ethenyl]-8-[(3, 3,3-trifluoropropyl)amino]imidazo[1, 2-b]pyridazin-3-yl}-2-methylbenzamide

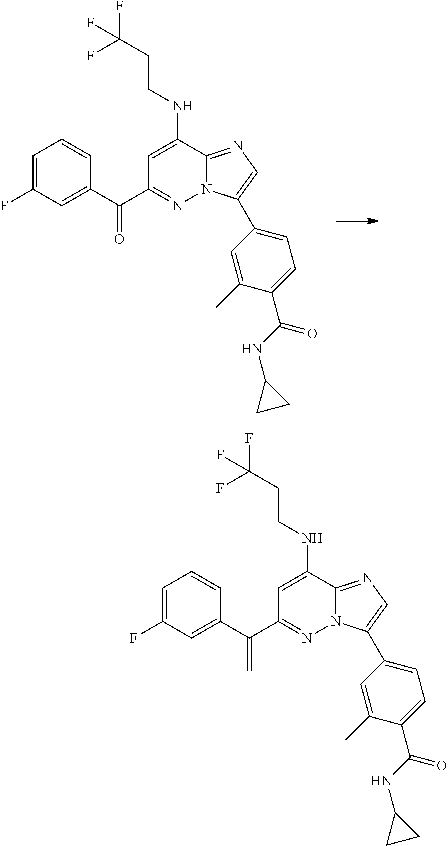

80.0 mg (152 μmol) N-cyclopropyl-4-{6-(3-fluorobenzoyl)-8-[(3,3,3-trifluoropropyl)amino]imidazo[1,2-b]pyridazin-3-yl}-2-methylbenzamide which was prepared according to comparative example 3b were transformed in analogy to the preparation method described for compound A1 to give after working up and purification 27 mg (33.7%) of the title compound.

¹H-NMR (DMSO-d₆): δ=0.44-0.51 (2H), 0.60-0.66 (2H), 2.16 (3H), 2.59-2.72 (2H), 2.77 (1H), 3.62 (2H), 5.81 (1H), 6.06 (1H), 6.41 (1H), 7.15-7.23 (2H), 7.23-7.30 (2H), 7.38-7.47 (1H), 7.55 (1H), 7.77-7.83 (1H), 7.85 (1H), 8.01 (1H), 8.20 (1H) ppm.

### Compound A25

### N-cyclopropyl-4-{6-[1-(3-fluorophenyl)cyclopropyl]-8-[(3, 3,3-trifluoropropyl)amino]imidazo[1, 2-b]pyridazin-3-yl}-2-methylbenzamide

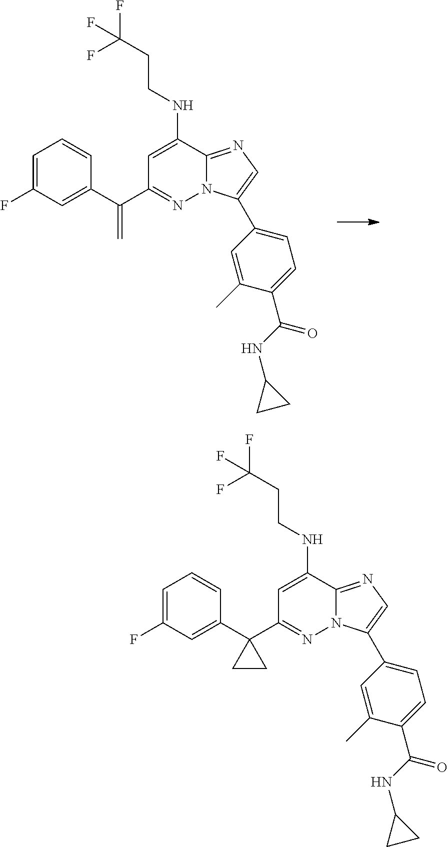

27.0 mg (52 μmol) N-cyclopropyl-4-{6-[1-(3-fluorophenyl)ethenyl]-8-[(3,3,3-trifluoropropyl)amino]imidazo[1,2-b]pyridazin-3-yl}-2-methylbenzamide which was prepared according to the preparation method described for compound A23 were transformed in analogy to compound A1 to give after working up and purification 9 mg (32%) of the title compound.

¹H-NMR (DMSO-d₆): δ=0.47-0.53 (2H), 0.61-0.69 (2H), 1.32-1.38 (2H), 1.54-1.59 (2H), 2.32 (3H), 2.57 (2H), 2.80 (1H), 3.51 (2H), 5.99 (1H), 7.03-7.11 (1H), 7.14-7.23 (2H), 7.27-7.40 (2H), 7.51 (1H), 7.84-7.91 (2H), 8.08 (1H), 8.26 (1H) ppm.

### Compound A26

### N-cyclopropyl-4-{6-(3-fluoro-4-methoxyphenoxy)-8-[(oxetan-3-ylmethyl)amino]imidazo[1, 2-b]pyridazin-3-yl}-2-methylbenzamide

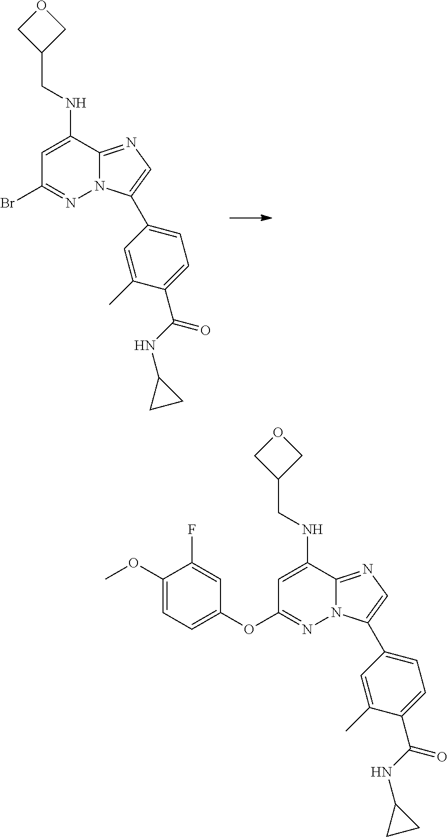 [SMILES: Cc1cc(-c2cnc3c(NCC4COC4)cc(Br)nn23)ccc1C(=O)NC1CC1 ; COc1ccc(Oc2cc(NCC3COC3)c3ncc(-c4ccc(C(=O)NC5CC5)c(C)c4)n3n2)cc1F]

A solution of 128 mg (900 μmol) of 3-fluoro-4-methoxyphenol in 2 mL of dimethylsulfoxide was treated with 36 mg (900 μmol) of sodium hydride and stirred at room temperature for 1 hour. Then 68 mg (150 μmol) of 4-{6-bromo-8-[(oxetan-3-ylmethyl)amino]imidazo[1,2-b]pyridazin-3-yl}-N-cyclopropyl-2-methylbenzamide, which was prepared according to intermediate example 26a, was added and the mixture was heated for 1 h at 130° C. and overnight at 120° C. to give after HPLC purification 17 mg (20%) of the title compound.

¹H-NMR (DMSO-d6): δ=0.43-0.50 (2H), 0.59-0.68 (2H), 2.11 (3H), 2.77 (1H), 3.35 (1H), 3.61 (2H), 3.83 (3H), 4.34 (2H), 4.63 (2H), 6.09 (1H), 7.02-7.09 (1H), 7.13-7.25 (2H), 7.30 (1H), 7.61-7.68 (1H), 7.77 (1H), 7.83 (1H), 7.91 (1H), 8.22 (1H) ppm.

### Intermediate Example 26a

### 4-{6-bromo-8-[(oxetan-3-ylmethyl)amino]imidazo[1,2-b]pyridazin-3-yl}-N-cyclopropyl-2-methylbenzamide

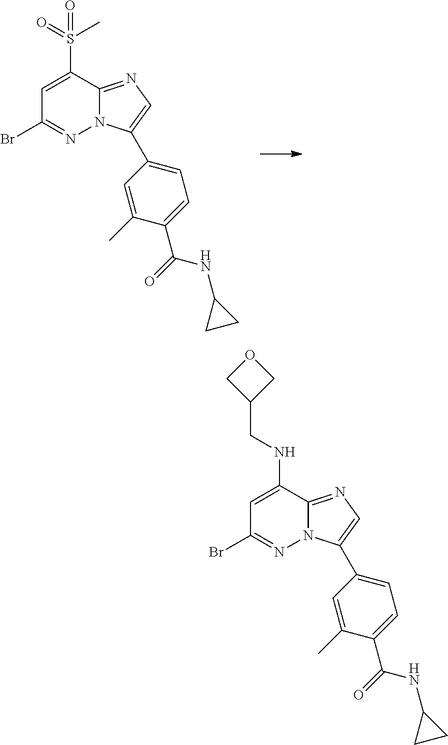

To a suspension of 3000 mg (6677 μmol) 4-[6-bromo-8-(methylsulfonyl)imidazo[1,2-b]pyridazin-3-yl]-N-cyclopropyl-2-methylbenzamide, which was prepared according to intermediate example 26b in 150 mL of THF were added 873 mg (1002 μmol) 1-(oxetan-3-yl)methanamine and 2589 mg (2003 μmol) DIPEA and the mixture was heated for 72 hours at 60° C. After further addition of 100 mg 1-(oxetan-3-yl)methanamine and heating for 8 h at 60° C., the solvent was removed in vaccuo and the residue was taken up in ethyl acetate and washed with water. The precipitate formed was filtered off to yield 0.99 g (33%) of the title compound. The remaining aqueous phase was reextracted with DCM, and the combined organic phases were evaporated. The residue was triturated with THF at 75° C. to yield another 1.65 g (53%) of the title compound.

¹H-NMR (DMSO-d6): δ=0.46-0.53 (2H), 0.61-0.69 (2H), 2.35 (3H), 2.75-2.85 (1H), 3.25 (1H), 3.54-3.69 (2H), 4.31 (2H), 4.62 (2H), 6.45 (1H), 7.36 (1H), 7.84 (1H), 7.90 (1H), 7.94 (1H), 8.12 (1H), 8.30 (1H) ppm.

### Intermediate Example 26b

### 4-[6-bromo-8-(methylsulfonyl)imidazo[1,2-b]pyridazin-3-yl]-N-cyclopropyl-2-methylbenzamide

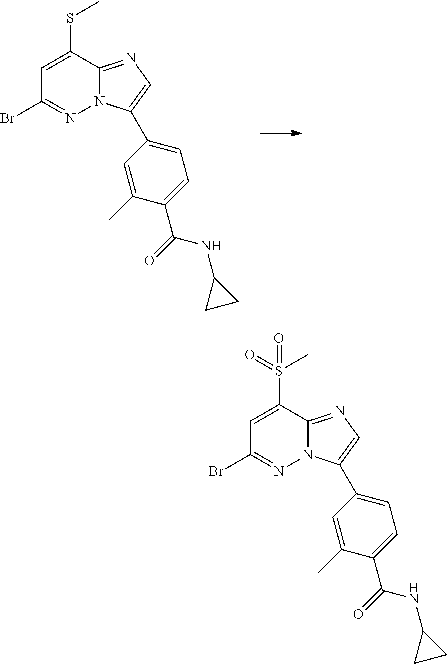 [SMILES: CSc1cc(Br)nn2c(-c3ccc(C(=O)NC4CC4)c(C)c3)cnc12 ; Cc1cc(-c2cnc3c(S(C)(=O)=O)cc(Br)nn23)ccc1C(=O)NC1CC1]

To a solution of 12.5 g (28.75 mmol) 4-[6-bromo-8-(methylsulfanyl)imidazo[1,2-b]pyridazin-3-yl]-N-cyclopropyl-2-methylbenzamide, which was prepared according to intermediate example 26c in 400 mL of DMF were added 53.03 g (86.26 mmol) potassium hydrogen sulfate sulfate (hydroperoxysulfonyl)oxidanide (5:1:1:2) and the mixture was stirred overnight at rt. to give, after aqueous work-up, 8.6 g (60%) of the title compound (impurity is 4-[6-bromo-8-(methylsulfinyl)imidazo[1,2-b]pyridazin-3-yl]-N-cyclopropyl-2-methylbenzamide) UPLC-MS: RT=0.98 min; m/z (ES+) 450.3 [MH+]; required MW=449.3.

### Intermediate Example 26c

### 4-[6-bromo-8-(methylsulfanyl)imidazo[1,2-b]pyridazin-3-yl]-N-cyclopropyl-2-methylbenzamide

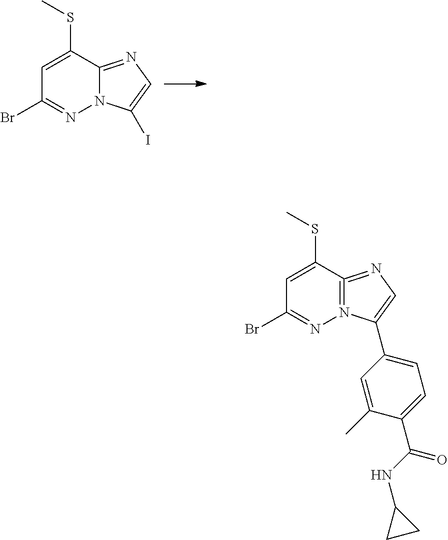

A mixture comprising 55.69 g (150 mmol) 6-bromo-3-iodo-8-(methylsulfanyl)imidazo[1,2-b]pyridazine which was prepared according to intermediate example 26d, 68 g (225 mmol) N-cyclopropyl-2-methyl-4-(4,4,5,5-tetramethyl-1,3,2-dioxaborolan-2-yl)benzamide, which was prepared according to intermediate example 26g, 11 g (15 mmol) (1,1,-bis(diphenylphosphino)ferrocene)-dichloropalladium (11), 450 mL aqueous 1M potassium carbonate solution and 632 mL tetrahydrofuran was stirred at 60° C. for 12 hours to give, after aqueous workup, 130 g crude product. The residue was triturated with DCM to yield 15.15 g (24%) of the title compound. The filtrate was purified by chromatography to give another 2.65 g (3%) of the title compound.

¹H-NMR (DMSO-d6): δ=0.47-0.54 (2H), 0.62-0.71 (2H), 2.36 (3H), 2.63 (3H), 2.77-2.85 (1H), 7.17 (1H), 7.40 (1H), 7.85 (1H), 7.90 (1H), 8.12 (1H), 8.31 (1H) ppm.

### Intermediate Example 26d

### 6-bromo-3-iodo-8-(methylsulfanyl)imidazo[1,2-b]pyridazine

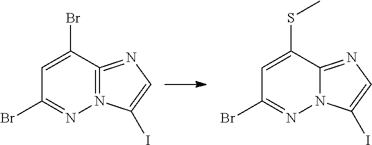

To a solution of 174 g (432 mmol) 6,8-dibromo-3-iodoimidazo[1,2-b]pyridazine which was prepared according to intermediate example 26e in 3.8 L dioxane were added 30.28 g (432 mmol) sodium methanethiolate and the mixture was stirred at 60° C. for 5 days. Further 25 g sodium methanethiolate were added and the mixture was stirred at 80° C. for 2 h. After cooling, the solution was poured on 4 L water Water and the aqueous phase was extracted with ethyl acetate. The organic phase was washed with water, dried over sodium sulphate, filtered and evaporated to give 102 g (64%) of the title compound.

¹H-NMR (DMSO-d6): δ=6.79 (1H), 7.67 (1H) ppm.

### Intermediate Example 26e

### 6,8-dibromo-3-iodoimidazo[1,2-b]pyridazine

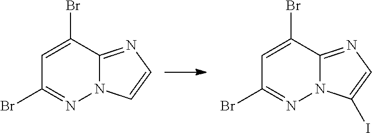 [SMILES: Brc1cc(Br)c2nccn2n1 ; Brc1cc(Br)c2ncc(I)n2n1]

To a mixture comprising 156 g (563 mmol) 6,8-dibromoimidazo[1,2-b]pyridazine which was prepared according to intermediate example 26f, 190 g (1637 mmol) N-iodosuccinimide and 1.3 L chloroform were added 5.5 mL HCL conc and the suspension was heated at 70° C. overnight. The precipitate was filtered off and triturated with diisopropylether to give 119 g (52%) of the title compound.

¹H-NMR (DMSO-d6): δ=7.92 (1H), 8.00 (1H) ppm.

### Intermediate Example 26f

### 6,8-dibromoimidazo[1,2-b]pyridazine

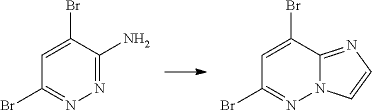 [SMILES: Nc1nnc(Br)cc1Br ; Brc1cc(Br)c2nccn2n1]

A mixture comprising 235 g (931 mmol) 4,6-dibromopyridazin-3-amine which was prepared according to intermediate example 26g, 421 mL (2792 mmol) 2-bromo-1,1-diethoxyethane, 2.93 L water and 227 mL THF was heated at 125° C. for h and at rt overnight. The solution was neutralized by addition of solid NaHCO₃, the precipitate was filtered off, washed with water and dried to give 156 g (80%) of the title compound as a brownish solid.

¹H-NMR (DMSO-d6): δ=7.81 (1H), 8.40 (1H) ppm.

### Intermediate Example 26g

### 6,8-dibromoimidazo[1,2-b]pyridazine

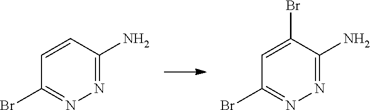 [SMILES: Nc1ccc(Br)nn1 ; Nc1nnc(Br)cc1Br]

To a mixture comprising 285 g (1638 mmol) 6-bromopyridazine-3-amine which was prepared according to intermediate example 26h, 275 g (3276 mmol) NaHCO₃ and 2815 mL MeOH was dropwise added 85 mL (1638 mmol) bromine at rt and it was stirred at rt overnight. After further addition of 34 mL (655 mmol) bromine and 55 g (655 mmol) NaHCO₃, the mixture was stirred overnight again.

The solvent was reduced to about 1000 mL and the mixture was poured on 5 L of water. The precipitate was filtered off, washed with water and dried give 411 g (99%) of the title compound.

¹H-NMR (CDCl₃): δ=6.14 (1H), 9.92 (2H) ppm.

### Intermediate Example 26h

### 6-bromopyridazin-3-amine

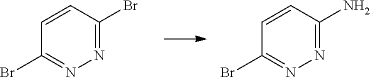 [SMILES: Brc1ccc(Br)nn1 ; Nc1ccc(Br)nn1]

A solution of 250 g (1.05 mol) 3,6-dibromopyridazine in 1.2 L 25% aqueous ammonia was heated to 100° C. at 11.7 bar overnight in an autoclave. After cooling, the precipitate was filtered off, washed with water and dried to give 137 g (75%) of the title compound.

¹H-NMR (DMSO-d6): δ=6.58 (1H), 6.69 (2H), 7.41 (1H) ppm.

### Intermediate Example 26i

### N-cyclopropyl-2-methyl-4-(4,4,5,5-tetramethyl-1,3,2-dioxaborolan-2-yl)benzamide

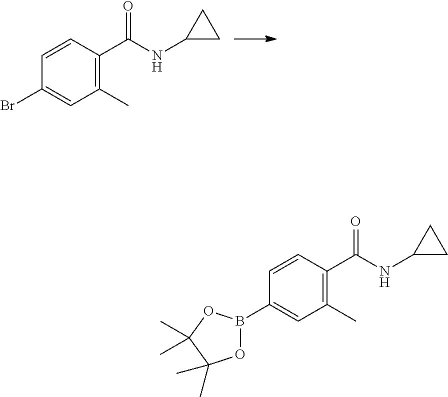 [SMILES: Cc1cc(Br)ccc1C(=O)NC1CC1 ; Cc1cc(B2OC(C)(C)C(C)(C)O2)ccc1C(=O)NC1CC1]

To a solution of 260 g (1.02 mol) 4-bromo-N-cyclopropyl-2-methylbenzamide which was prepared according to intermediate example 26j in 2 L dioxane at 23° C. were added 390 g bis-(pinacolato)-diboron, 19.5 g 2-dicyclohexylphosphino-2′,4′,6′-triisopropylbiphenyl, 150 .g potassium acetate and 9.37 g tris-(dibenzylidenaceton)-dipalladium(0) and the mixture was refluxed for 6 h. After cooling to 23° C., water and ethyl acetate were added and the mixture stirred for 15 min. The organic phase was washed with water, dried over sodium sulfate, filtered and evaporated. The residue was purified by chromatography to give 308 g (56%) of the title compound.

¹H-NMR (300 MHz, CDCl₃): δ=0.59 (2H), 0.85 (2H), 1.33 (6H), 2.41 (3H), 2.87 (1H), 5.94 (1H), 7.28 (1H), 7.60 (1H), 7.63 (1H) ppm.

### Intermediate Example 26j

### 4-Bromo-N-cyclopropyl-2-methylbenzamide

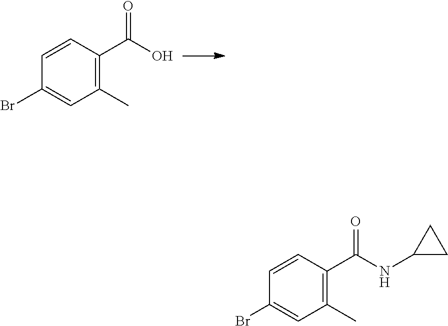

To a stirred solution of 300 g (1.4 mol) 4-bromo-2-methylbenzoic acid in 8.4 L dichloromethane at 23° C. were added 79.6 g cyclopropanamine and 320.9 g EDC. After stirring overnight, the solution was washed with water and the aqueous phase was extracted with dichloromethane. The combined organic phases were dried over sodium sulfate, filtered and evaporated. The remaining solid was triturated with diisopropyl ether, filtered, washed and dried in vaccuo to yield 260 g (73%) of the title compound.

### Compound A27

### N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide

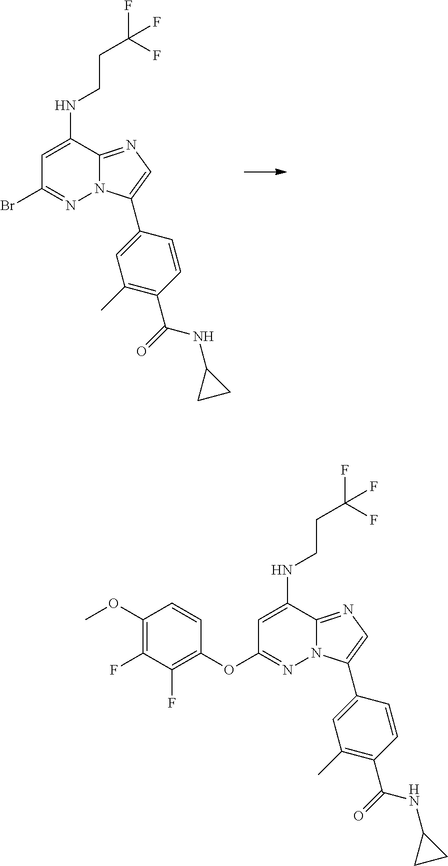 [SMILES: Cc1cc(-c2cnc3c(NCCC(F)(F)F)cc(Br)nn23)ccc1C(=O)NC1CC1 ; COc1ccc(Oc2cc(NCCC(F)(F)F)c3ncc(-c4ccc(C(=O)NC5CC5)c(C)c4)n3n2)c(F)c1F]

A solution of 31.9 g (199 mmol) 2,3-difluoro-4-methoxyphenol in 450 mL of dimethylsulfoxide was treated with 7.96 g (199 mmol) of sodium hydride and stirred at room temperature for 1 hour. Then 16 g (33.2 mmol) of 4-{6-bromo-8-[(3,3,3-trifluoropropyl)amino]imidazo[1,2-b]pyridazin-3-yl}-N-cyclopropyl-2-methylbenzamide, which was prepared according to intermediate example 27a, was added and the mixture was heated overnight at 130° C. After cooling, 300 mL ethyl acetate were added and the organic phase is washed with water. After evaporation of the organic phase, the residue was triturated with 200 mL ethanol to give 12.05 g (65%) of the title compound.

¹H-NMR (DMSO-d6): δ=0.47-0.53 (2H), 0.62-0.70 (2H), 2.11 (3H), 2.72 (2H), 2.80 (1H), 3.64 (2H), 3.92 (3H), 6.22 (1H), 7.12 (1H), 7.18 (1H), 7.27 (1H), 7.63 (1H), 7.72 (1H), 7.75-7.81 (1H), 7.97 (1H), 8.24 (1H) ppm.

### Intermediate Example 27a

### 4-{6-Bromo-8-[(3,3,3-trifluoropropyl)amino]imidazo[1,2-b]pyridazin-3-yl}-N-cyclopropyl-2-methylbenzamide

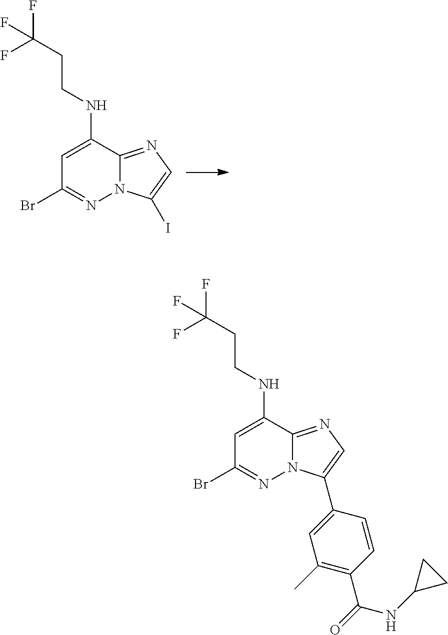 [SMILES: FC(F)(F)CCNc1cc(Br)nn2c(I)cnc12 ; Cc1cc(-c2cnc3c(NCCC(F)(F)F)cc(Br)nn23)ccc1C(=O)NC1CC1]

A mixture comprising 127 g (292 mmol) 6-bromo-3-iodo-N-(3,3,3-trifluoropropyl)imidazo[1,2-b]pyridazin-8-amine which was prepared according to intermediate example 27b, 95.93 g (438 mmol) [4-(cyclopropylcarbamoyl)-3-methylphenyl]boronic acid which was prepared according to intermediate example 27c, 23.8 g (29 mmol) (1,1,-bis(diphenylphosphino)ferrocene)-dichloropalladium (11), 438 mL aqueous 1M potassium carbonate solution and 973 mL tetrahydrofuran was stirred at 80° C. for 8 hours and further 6 days at 60° C. Ethyl acetate was added to the separated organic phase and the mixture was washed with water. After filtration over ALLOX, the organic phase was evaporated and the residue was triturated with 200 mL ethanol to give 71.2 g (51%) of the title compound.

¹H-NMR (DMSO-d6): δ=0.48-0.58 (2H), 0.63-0.73 (2H), 2.38 (3H), 2.68 (2H), 2.83 (1H), 3.61 (2H), 6.49 (1H), 7.40 (1H), 7.87 (1H), 7.93 (1H), 7.96-8.04 (2H), 8.32 (1H) ppm.

### Intermediate Example 27b

### 6-Bromo-3-iodo-N-(3,3,3-trifluoropropyl)imidazo[1,2-b]pyridazin-8-amine

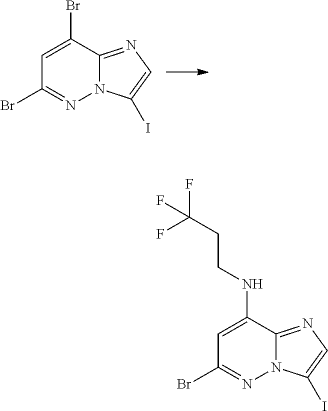 [SMILES: Brc1cc(Br)c2ncc(I)n2n1 ; FC(F)(F)CCNc1cc(Br)nn2c(I)cnc12]

To a solution of 119 g 295 mmol) 6,8-dibromo-3-iodoimidazo[1,2-b]pyridazine which was prepared according to intermediate example 26e in 800 mL THF were added 66.8 g (590.8 mmol) 3,3,3-trifluoropropan-1-amine and the mixture was stirred at 80° c. for 2 h and at 50° C. overnight. The solution was evaporated, 600 mL ethyl acetate were added and the mixture was washed with water. The organic phase was dried and evaporated to give 127 g (99%) of the title compound.

¹H-NMR (DMSO-d6): δ=2.63 (2H), 3.55 (2H), 6.43 (1H), 7.59 (1H), 7.89-7.98 (1H) ppm.

### Intermediate Example 27c

### [4-(cyclopropylcarbamoyl)-3-methylphenyl]boronic acid

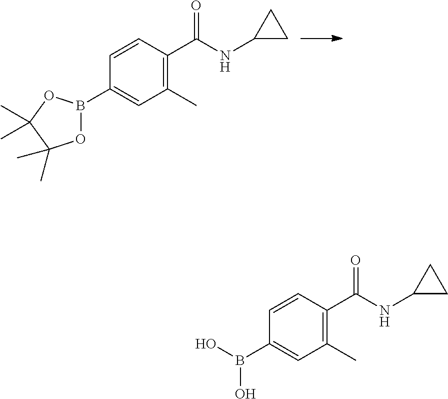 [SMILES: Cc1cc(B2OC(C)(C)C(C)(C)O2)ccc1C(=O)NC1CC1 ; Cc1cc(B(O)O)ccc1C(=O)NC1CC1]

To a solution of N-cyclopropyl-2-methyl-4-(4,4,5,5-tetramethyl-1,3,2-dioxaborolan-2-yl)benzamide (20.2 g, 67.13 mol) which was prepared according to intermediate example 26i in acetone (300 mL) at rt was added sodium periodate (43.1 g, 201.40 mol) and ammonium acetate (134.26 mol, 134 mL 1M aqueous solution) and the mixture was stirred for 3 h. More water was added (120 mL), and the mixture was stirred at 40° C. for 2 h more. After addition of 4 N HCL (32 mL), the organic phase was removed in vaccuo and the reminder was extracted with ethyl actate. The organic phase was washed with sat. sodium chloride solution, filtered through a Whatman filter and evaporated. The residue was redissolved in toluene and evaporated (two times) to yield 14.59 g (94.3%) [4-(cyclopropylcarbamoyl)-3-methylphenyl]boronic acid: ¹H-NMR (300 MHz, d₆-DMSO): 6=8.21 (1H), 8.04 (2H), 7.56 (2H), 7.17 (1H), 2.77 (1H), 2.25 (3H), 0.62 (2H), 0.47 (2H) ppm.

### Compound A28

### N-cyclopropyl-4-{6-(2,3-difluoro-4-methoxyphenoxy)-8-[(tetrahydro-2H-pyran-4-ylmethyl)amino]imidazo[1, 2-b]pyridazin-3-yl}-2-methylbenzamide

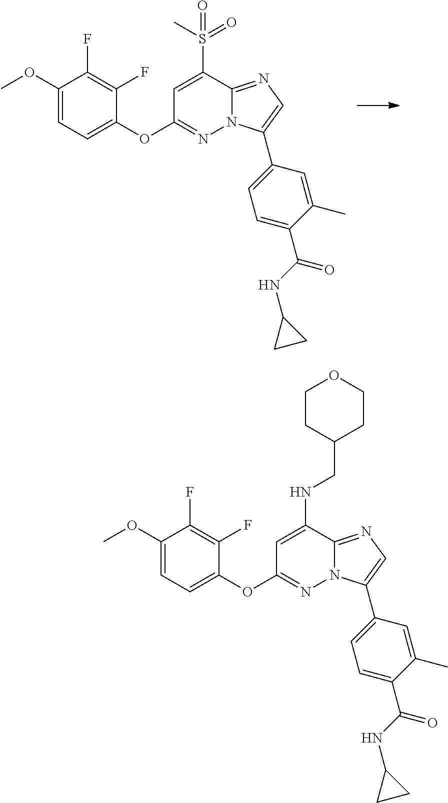

To a solution of 52 mg (0.1 mmol) N-cyclopropyl-4-[6-(2,3-difluoro-4-methoxyphenoxy)-8-(methylsulfonyl)imidazo[1,2-b]pyridazin-3-yl]-2-methylbenzamide which was prepared according to intermediate example 28a in NMP (2 mL) at rt was 1-(tetrahydro-2H-pyran-4-yl)methanamine (35 mg, 0.3 mmol) and DIPEA (0.3 mmol, 51 μL) and the mixture was stirred at 110° C. for 72 h to give after HPLC purification 26.9 mg (47%) of the title compound ¹H-NMR (DMSO-d6): δ=0.43-0.50 (2H), 0.58-0.69 (2H), 1.22 (2H), 1.62 (2H), 1.86-2.02 (1H), 2.04-2.11 (3H), 2.77 (1H), 3.19-3.30 (4H), 3.82 (2H), 3.89 (3H), 6.16 (1H), 7.03-7.13 (1H), 7.15 (1H), 7.20-7.30 (1H), 7.57-7.63 (1H), 7.69 (1H), 7.80 (1H), 7.93 (1H), 8.24 (1H) ppm.

### Intermediate Example 28a

### N-cyclopropyl-4-[6-(2,3-difluoro-4-methoxyphenoxy)-8-(methylsulfonyl)imidazo[1,2-b]pyridazin-3-yl]-2-methylbenzamide

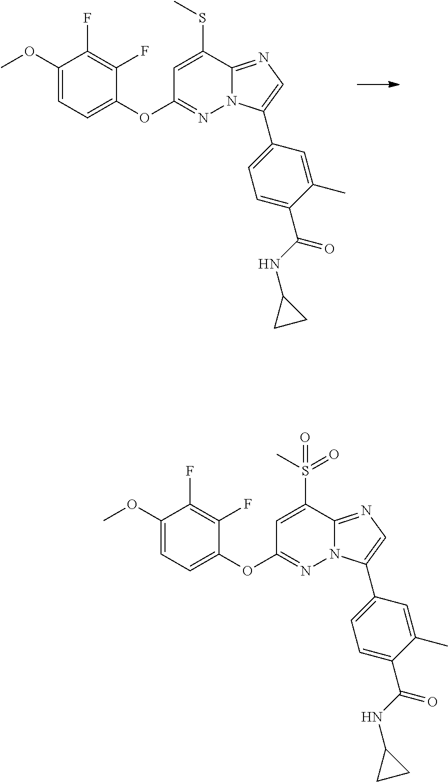

986 mg (1986 μmol) N-cyclopropyl-4-[6-(2,3-difluoro-4-methoxyphenoxy)-8-(methylsulfanyl)imidazo[1,2-b]pyridazin-3-yl]-2-methylbenzamide which was prepared according to intermediate example 28b were transformed in analogy to intermediate example 26b to give after working up 1040 mg (99%) of the title compound.

¹H-NMR (DMSO-d6): δ=0.44-0.51 (2H), 0.61-0.69 (2H), 2.11 (3H), 2.73-2.84 (1H), 3.66 (3H), 3.91 (3H), 7.13-7.21 (1H), 7.23 (1H), 7.33-7.42 (1H), 7.64-7.70 (3H), 8.30 (1H), 8.40 (1H) ppm.

### Intermediate Example 28b

### N-cyclopropyl-4-[6-(2,3-difluoro-4-methoxyphenoxy)-8-(methylsulfanyl)imidazo[1,2-b]pyridazin-3-yl]-2-methylbenzamide

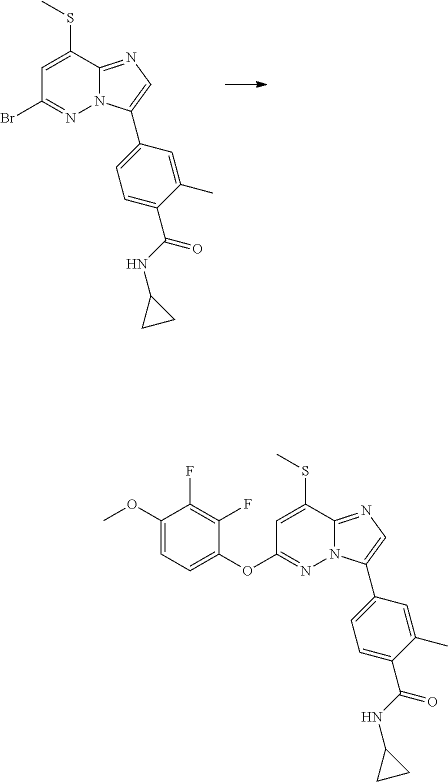

3.1 g (7428 μmol) 4-[6-bromo-8-(methylsulfanyl)imidazo[1,2-b]pyridazin-3-yl]-N-cyclopropyl-2-methylbenzamide which was prepared according to intermediate example 26b were transformed in analogy to compound A27 using 2,3-difluoro-4-methoxyphenol to give after working up and purification 1010 mg (27%) of the title compound.

¹H-NMR (DMSO-d6): δ=0.44-0.50 (2H), 0.59-0.70 (2H), 2.09 (3H), 2.67 (3H), 2.73-2.83 (1H), 3.90 (3H), 7.06 (1H), 7.14 (1H), 7.19 (1H), 7.31 (1H), 7.58-7.65 (1H), 7.67 (1H), 8.09 (1H), 8.26 (1H) ppm.

### Proliferation Assay

Cultivated tumour cells (MCF7, hormone dependent human mammary carcinoma cells, ATCC HTB22; NCI-H460, human non-small cell lung carcinoma cells, ATCC HTB-177; DU 145, hormone-independent human prostate carcinoma cells, ATCC HTB-81; HeLa-MaTu, human cervical carcinoma cells, EPO-GmbH, Berlin; HeLa-MaTu-ADR, multidrug-resistant human cervical carcinoma cells, EPO-GmbH, Berlin; HeLa human cervical tumour cells, ATCC CCL-2; B16F10 mouse melanoma cells, ATCC CRL-6475) were plated at a density of 5000 cells/well (MCF7, DU145, HeLa-MaTu-ADR), 3000 cells/well (NCI-H460, HeLa-MaTu, HeLa), or 1000 cells/well (B16F10) in a 96-well multititer plate in 200 μL of their respective growth medium supplemented 10% fetal calf serum.

After 24 hours, the cells of one plate (zero-point plate) were stained with crystal violet (see below), while the medium of the other plates was replaced by fresh culture medium (200 μl), to which the test substances were added in various concentrations (0 μM, as well as in the range of 0.01-30 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were stained by adding 100 μl/measuring point of a 0.1% crystal violet solution (pH 3.0). After three washing cycles of the stained cells with water, the plates were dried at room temperature. The dye was dissolved by adding 100 μl/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μm) cells (=100%). The IC50 values were determined by means of a 4 parameter fit using the company's own software.

### Mps-1 Kinase Assay

The human kinase Mps-1 phosphorylates a biotinylated substrate peptide. Detection of the phosphorylated product is achieved by time-resolved fluorescence resonance energy transfer (TR-FRET) from Europium-labelled anti-phospho-Serine/Threonine antibody as donor to streptavidin Labelled with cross-linked allophycocyanin (SA-XLent) as acceptor. Compounds are tested for their inhibition of the kinase activity.

N-terminally GST-tagged human full length recombinant Mps-1 kinase (purchased from Invitrogen, Karslruhe, Germany, cat. no PV4071) was used. As substrate for the kinase reaction a biotinylated peptide of the amino-acid sequence PWDPDDADITEILG (C-terminus in amide form, purchased from Biosynthan GmbH, Berlin) was used.

For the assay 50 nL of a 100-fold concentrated solution of the test compound in DMSO was pipetted into a black low volume 384well microtiter plate (Greiner Bio-One, Frickenhausen, Germany), 2 μl of a solution of Mps-1 in assay buffer [0.1 mM sodium-ortho-vanadate, 10 mM MgCl₂, 2 mM DTT, 25 mM Hepes pH 7.7, 0.05% BSA, 0.001% Pluronic F-127] were added and the mixture was incubated for 15 min at 22° C. to allow pre-binding of the test compounds to Mps-1 before the start of the kinase reaction. Then the kinase reaction was started by the addition of 3 μl of a solution of 16.7 adenosine-tri-phosphate (ATP, 16.7 μM=>final conc. in the 5 μl assay volume is 10 μM) and peptide substrate (1.67 μM=>final conc. in the 5 μl assay volume is 1 μM) in assay buffer and the resulting mixture was incubated for a reaction time of 60 min at 22° C. The concentration of Mps-1 in the assay was adjusted to the activity of the enzyme lot and was chosen appropriate to have the assay in the linear range, typical enzyme concentrations were in the range of about 1 nM (final conc. in the 5 μl assay volume). The reaction was stopped by the addition of 3 μl of a solution of HTRF detection reagents (100 mM Hepes pH 7.4, 0.1% BSA, 40 mM EDTA, 140 nM Streptavidin-XLent [#61GSTXLB, Fa. Cis Biointernational, Marcoule, France], 1.5 nM anti-phospho(Ser/Thr)-Europium-antibody [#AD0180, PerkinElmer LAS, Rodgau-Jügesheim, Germany].

The resulting mixture was incubated 1 h at 22° C. to allow the binding of the phosphorylated peptide to the anti-phospho(Ser/Thr)-Europium-antibody. Subsequently the amount of phosphorylated substrate was evaluated by measurement of the resonance energy transfer from the Europium-labelled anti-phospho(Ser/Thr) antibody to the Streptavidin-XLent. Therefore, the fluorescence emissions at 620 nm and 665 nm after excitation at 350 nm was measured in a Viewlux TR-FRET reader (PerkinElmer LAS, Rodgau-Jügesheim, Germany). The “blank-corrected normalized ratio” (a Viewlux specific readout, similar to the traditional ratio of the emissions at 665 nm and at 622 nm, in which blank and Eu-donor crosstalk are subtracted from the 665 nm signal before the ratio is calculated) was taken as the measure for the amount of phosphorylated substrate. The data were normalised (enzyme reaction without inhibitor=0% inhibition, all other assay components but no enzyme=100% inhibition). Test compounds were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100fold conc. stock solutions by serial 1:3 dilutions) in duplicate values for each concentration and IC₅₀ values were calculated by a 4 parameter fit using an in-house software.

TABLE 1

| | Mps-1 |
|---|---|
| Example | IC₅₀ [nM] |
| A1 | 0.4 |
| A2 | 0.6 |
| A3 | 0.2 |
| A4 | 0.3 |
| A5 | 0.7 |
| A6 | 0.7 |
| A7 | 0.5 |
| A8 | 0.8 |
| A9 | 1.2 |
| A10 | 0.5 |
| A11 | 0.3 |
| A12 | 0.6 |
| A13 | 0.4 |
| A14 | 0.6 |
| A15 | 0.4 |
| A16 | 0.6 |
| A17 | 0.7 |
| A18 | 1.5 |
| A19 | 0.6 |
| A20 | 0.8 |
| A21 | 0.5 |
| A22 | 0.4 |
| A23 | 0.4 |
| A24 | 0.3 |
| A25 | 0.9 |

### Spindle Assembly Checkpoint Assay

The spindle assembly checkpoint assures the proper segregation of chromosomes during mitosis. Upon entry into mitosis, chromosomes begin to condensate which is accompanied by the phosphorylation of histone H3 on serine 10. Dephosphorylation of histone H3 on serine 10 begins in anaphase and ends at early telophase. Accordingly, phosphorylation of histone H3 on serine 10 can be utilized as a marker of cells in mitosis. Nocodazole is a microtubule destabilizing substance. Thus, nocodazole interferes with microtubule dynamics and mobilises the spindle assembly checkpoint. The cells arrest in mitosis at G2/M transition and exhibit phosphorylated histone H3 on serine 10. An inhibition of the spindle assembly checkpoint by Mps-1 inhibitors overrides the mitotic blockage in the presence of nocodazole, and the cells complete mitosis prematurely. This alteration is detected by the decrease of cells with phosphorylation of histone H3 on serine 10. This decline is used as a marker to determine the capability of compounds of the present invention to induce a mitotic breakthrough.

Cultivated cells of the human cervical tumour cell line HeLa (ATCC CCL-2) were plated at a density of 2500 cells/well in a 384-well microtiter plate in 20 μl Dulbeco's Medium (w/o phenol red, w/o sodium pyruvate, w 1000 mg/ml glucose, w pyridoxine) supplemented with 1% (v/v) glutamine, 1% (v/v) penicillin, 1% (v/v) streptomycin and 10% (v/v) fetal calf serum. After incubation overnight at 37° C., 10 μl/well nocodazole at a final concentration of 0.1 μg/ml were added to cells. After 24 h incubation, cells were arrested at G2/M phase of the cell cycle progression. Test compounds solubilised in dimethyl sulfoxide (DMSO) were added at various concentrations (0 μM, as well as in the range of 0.005 μM-10 μM; the final concentration of the solvent DMSO was 0.5% (v/v)). Cells were incubated for 4 h at 37° C. in the presence of test compounds. Thereafter, cells were fixed in 4% (v/v) paraformaldehyde in phosphate buffered saline (PBS) at 4° C. overnight then permeabilised in 0.1% (v/v) Triton X™ 100 in PBS at room temperature for 20 min and blocked in 0.5% (v/v) bovine serum albumin (BSA) in PBS at room temperature for 15 min. After washing with PBS, 20 μl/well antibody solution (anti-phospho-histone H3 clone 3H10, FITC; Upstate, Cat#16-222; 1:200 dilution) was added to cells, which were incubated for 2 h at room temperature. Afterwards, cells were washed with PBS and 20 μl/well HOECHST 33342 dye solution (5 μg/ml) was added to cells and cells were incubated 12 min at room temperature in the dark. Cells were washed twice with PBS then covered with PBS and stored at 4° C. until analysis. Images were acquired with a Perkin Elmer OPERA™ High-Content Analysis reader. Images were analyzed with image analysis software MetaXpress™ from Molecular devices utilizing the Cell Cycle application module. In this assay both labels HOECHST 33342 and phosphorylated Histone H3 on serine 10 were measured. HOECHST 33342 labels DNA and is used to count cell number. The staining of phosphorylated Histone H3 on serine 10 determines the number of mitotic cells. Inhibition of Mps-1 decreases the number of mitotic cells in the presence of nocodazole indicating an inappropriate mitotic progression. The raw assay data were further analysed by four parameter logistic regression analysis to determine the IC₅₀ value for each tested compound.

Investigation of In Vitro Metabolic Stability in Rat Hepatocytes (Including Calculation of Hepatic In Vivo Blood Clearance (CL))

Hepatocytes from Han Wistar rats were isolated via a 2-step perfusion method. After perfusion, the liver was carefully removed from the rat: the liver capsule was opened and the hepatocytes were gently shaken out into a Petri dish with ice-cold WME. The resulting cell suspension was filtered through sterile gaze in 50 ml falcon tubes and centrifuged at 50×g for 3 min at room temperature.

The cell pellet was resuspended in 30 ml WME and centrifuged through a Percoll® gradient for 2 times at 100×g. The hepatocytes were washed again with Williams' medium E (WME) and resuspended in medium containing 5% FCS. Cell viability was determined by trypan blue exclusion.

For the metabolic stability assay liver cells were distributed in WME containing 5% FCS to glass vials at a density of 1.0×10⁶ vital cells/m. The test compound was added to a final concentration of 1 μM. During incubation, the hepatocyte suspensions were continuously shaken and aliquots were taken at 2, 8, 16, 30, 45 and 90 min, to which equal volumes of cold methanol were immediately added. Samples were frozen at −20° C. over night, after subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, amount of liver cells in vivo and in vitro. The hepatic in vivo blood clearance (CL) and the maximal oral bioavailability (Fₘₐₓ) was calculated. The following parameter values were used: Liver blood flow—4.2 L/h/kg rat; specific liver weight—32 g/kg rat body weight; liver cells in vivo—1.1×10⁸ cells/g liver, liver cells in vitro—0.5×106/ml.

Determination of Inhibitory Potential on Human CYP3A4

The potential of the test compound to act as a competitive inhibitor of CYP3A4 was evaluated in in vitro assays, using human liver microsomes and the reference substrate midazolam. The test compound was solved in acetonitrile. Human liver microsomal preparation (pool of HLM) was applied for the assay.

A stock solution of the test compound was added to phosphate buffer containing EDTA, NADP, glucose 6-phosphate, and glucose 6-phosphate dehydrogenase. This mixture was sequentially diluted on a Genesis Workstation (Tecan, Crailsheim, FRG). After pre-warming, reaction was initiated by addition of a mixture of probe substrate (midazolam). Finally, the incubation mixtures contained human liver microsomes at protein concentration of 60 μg/mL, NADPH-regenerating system (1 mM NADP, 5.0 mM glucose 6-phosphate, glucose 6-phosphate dehydrogenase (1.5 U/mL), 1.0 mM EDTA, the test compound at 6 different concentrations, 2.5 μM midazolam as probe substrate, and phosphate buffer (50 mM, pH 7.4) in a total volume of 200 μL. Incubations were performed on a Genesis Workstation (Tecan, Crailsheim, FRG) in 96-well plates (Microtiter plate, 96-well plate) at 37° C. Stock solution of probe substrate was prepared in water (midazolam 10 mM). Ketoconazole was used as positive control of a direct-acting inhibitor. The reference samples (substrate, but no inhibitor) were incubated in parallel in sextuple and contained the same amount of solvent as the test incubations. Reactions were stopped by addition of 100 μL acetonitrile containing the internal standard. Precipitated proteins were removed by centrifugation of the well plate, supernatants were analyzed by LC-MS/MS.

The CYP3A4-mediated metabolic activity in the presence of the test compounds was expressed as percentages of the corresponding reference value. A sigmoid-shaped curve was fitted to the data to calculate the enzyme inhibition parameter IC50 using a nonlinear least-squares regression analysis of the plot of percent control activity versus concentration of the test inhibitor. Observing less than 50% inhibition, the data were not extrapolated; hence, IC50 were reported as being greater than the highest concentration of the test compound applied.

Compounds A26, A27, and A28 are characterized by: activity in Spindle Assembly Checkpoint Assay <1.0 nM, activity in Proliferation Assay with HeLa cells <25 nM, in vitro metabolic stability in rat hepatocytes Fmax≧39%, and inhibition of liver enzyme CYP3A4≧5 μM.

Tables 2, 3, 4, 5 and 6 compare the in vitro metabolic stability in rat hepatocytes expressed as hepatic in vivo blood clearance (CL) and the maximal oral bioavailability (Fₘₐₓ) for three sets of compounds.

TABLE 2

| | 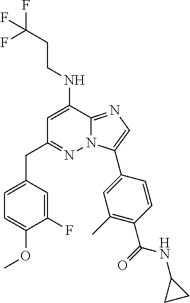 [SMILES: COc1ccc(Cc2cc(NCCC(F)(F)F)c3ncc(-c4ccc(C(=O)NC5CC5)c(C)c4)n3n2)cc1F] | 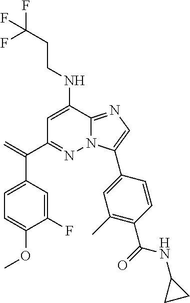 [SMILES: C=C(c1ccc(OC)c(F)c1)c1cc(NCCC(F)(F)F)c2ncc(-c3ccc(C(=O)NC4CC4)c(C)c3)n2n1] | 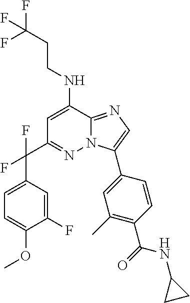 [SMILES: COc1ccc(C(F)(F)c2cc(NCCC(F)(F)F)c3ncc(-c4ccc(C(=O)NC5CC5)c(C)c4)n3n2)cc1F] | 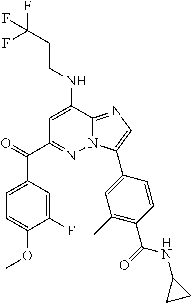 [SMILES: COc1ccc(C(=O)c2cc(NCCC(F)(F)F)c3ncc(-c4ccc(C(=O)NC5CC5)c(C)c4)n3n2)cc1F] |
|---|---|---|---|---|
| Example | Comparative Example 1 | Compound A1 | Compound A2 | Compound A13 |
| Fₘₐₓ [%] | 29 | 39 | 64 | 75 |
| CL [L/h/kg] | 3.0 | 2.6 | 1.5 | 1.1 |

TABLE 3

| | 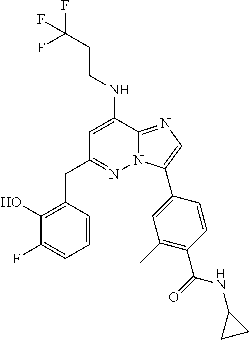 [SMILES: Cc1cc(-c2cnc3c(NCCC(F)(F)F)cc(Cc4cccc(F)c4O)nn23)ccc1C(=O)NC1CC1] | 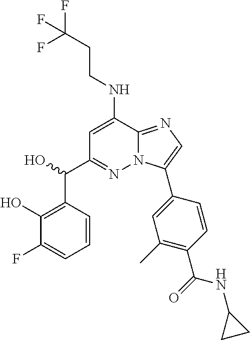 [SMILES: Cc1cc(-c2cnc3c(NCCC(F)(F)F)cc(C(O)c4cccc(F)c4O)nn23)ccc1C(=O)NC1CC1] | 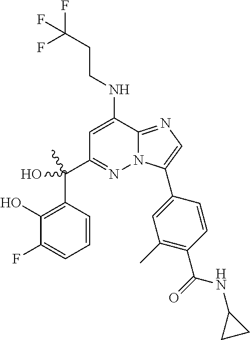 |
|---|---|---|---|
| Example | Comparative Example 2 | Compound A3 | Compound A4 |
| Fₘₐₓ [%] | 3 | 18 | 36 |
| CL [L/h/kg] | 4.1 | 3.4 | 2.7 |

TABLE 4

| | 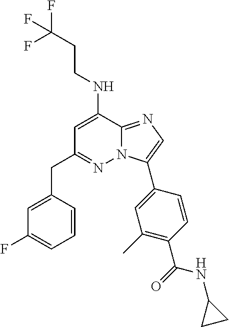 [SMILES: Cc1cc(-c2cnc3c(NCCC(F)(F)F)cc(Cc4cccc(F)c4)nn23)ccc1C(=O)NC1CC1] | 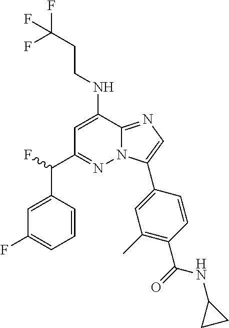 [SMILES: Cc1cc(-c2cnc3c(NCCC(F)(F)F)cc(C(F)c4cccc(F)c4)nn23)ccc1C(=O)NC1CC1] | 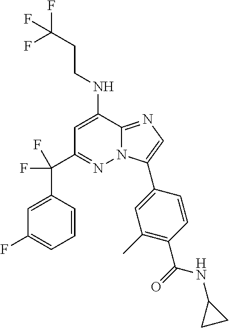 [SMILES: Cc1cc(-c2cnc3c(NCCC(F)(F)F)cc(C(F)(F)c4cccc(F)c4)nn23)ccc1C(=O)NC1CC1] |
|---|---|---|---|
| Example | Comparative Example 3 | Compound A5 | Compound A6 |
| Fₘₐₓ [%] | 43 | 49 | 70 |
| CL [L/h/kg] | 2.4 | 2.1 | 1.3 |
| | | 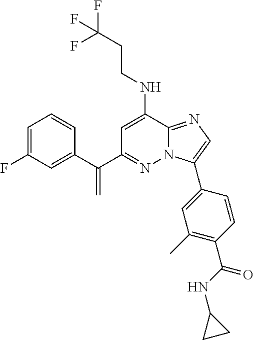 [SMILES: C=C(c1cccc(F)c1)c1cc(NCCC(F)(F)F)c2ncc(-c3ccc(C(=O)NC4CC4)c(C)c3)n2n1] | 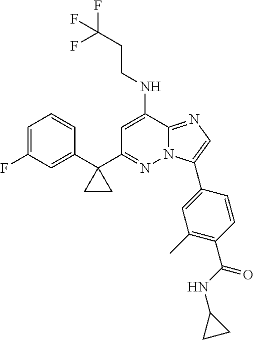 [SMILES: Cc1cc(-c2cnc3c(NCCC(F)(F)F)cc(C4(c5cccc(F)c5)CC4)nn23)ccc1C(=O)NC1CC1] |
| | Example | Compound A24 | Compound A25 |
| | Fₘₐₓ [%] | 48 | 52 |
| | CL [L/h/kg] | 2.2 | 2.0 |

TABLE 5

| | 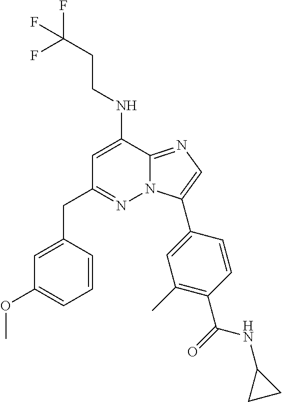 [SMILES: COc1cccc(Cc2cc(NCCC(F)(F)F)c3ncc(-c4ccc(C(=O)NC5CC5)c(C)c4)n3n2)c1] | 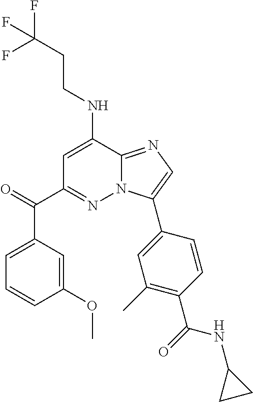 [SMILES: COc1cccc(C(=O)c2cc(NCCC(F)(F)F)c3ncc(-c4ccc(C(=O)NC5CC5)c(C)c4)n3n2)c1] | 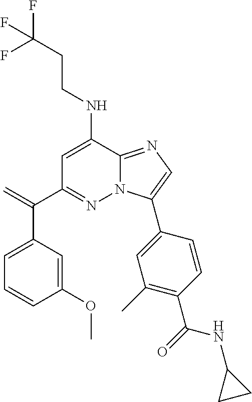 [SMILES: C=C(c1cccc(OC)c1)c1cc(NCCC(F)(F)F)c2ncc(-c3ccc(C(=O)NC4CC4)c(C)c3)n2n1] |
|---|---|---|---|
| Example | Comparative Example 4 | Compound A7 | Compound A8 |
| Fₘₐₓ [%] | 18 | 26 | 57 |
| CL [L/h/kg] | 3.4 | 3.1 | 1.8 |

TABLE 6

| | 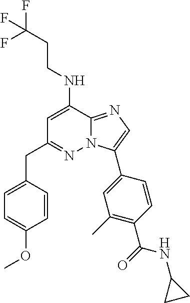 [SMILES: COc1ccc(Cc2cc(NCCC(F)(F)F)c3ncc(-c4ccc(C(=O)NC5CC5)c(C)c4)n3n2)cc1] | 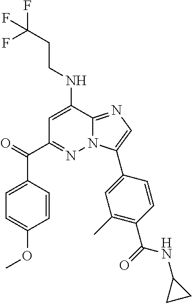 [SMILES: COc1ccc(C(=O)c2cc(NCCC(F)(F)F)c3ncc(-c4ccc(C(=O)NC5CC5)c(C)c4)n3n2)cc1] | 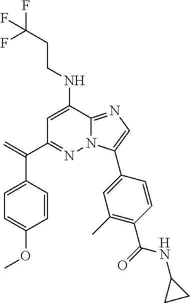 [SMILES: C=C(c1ccc(OC)cc1)c1cc(NCCC(F)(F)F)c2ncc(-c3ccc(C(=O)NC4CC4)c(C)c3)n2n1] | 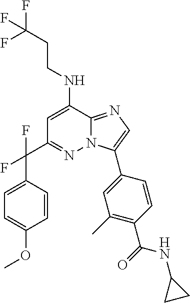 [SMILES: COc1ccc(C(F)(F)c2cc(NCCC(F)(F)F)c3ncc(-c4ccc(C(=O)NC5CC5)c(C)c4)n3n2)cc1] |
|---|---|---|---|---|
| Example | Comparative Example 5 | Compound A9 | Compound A10 | Compound A16 |
| Fₘₐₓ [%] | 35 | 71 | 48 | 56 |
| CL [L/h/kg] | 2.7 | 1.2 | 2.2 | 1.9 |

Table 7 lists hepatic in vivo blood clearance and the maximal oral bioavailability of additional compounds.

TABLE 7

| Example | Fₘₐₓ [%] | CL [L/h/kg] |
|---|---|---|
| A11 | 45 | 2.3 |
| A12 | 67 | 1.4 |
| A14 | 51 | 2.1 |
| A15 | 53 | 2.0 |
| A17 | 50 | 2.1 |
| A18 | 76 | 1.0 |
| A22 | 28 | 3.0 |
| A23 | 40 | 2.5 |

In Vivo Anti-Tumor Efficacy of Mps-1 Kinase Inhibitors of the Present Invention in Combination with Paclitaxel in A2780cis Human Ovarian Cancer Model in Nude Mice

The effect of combination treatment of an Mps-1 kinase inhibitor of the present invention and paclitaxel was studied in the adaptive cisplatin-resistant and paclitaxel intrinsically resistant A2780cis xenograft model. Compound A27 was applied orally upon sub-optimal doses (40% and 60% of MTD) in combination in the twice daily intermittent (2 days on/5 days off) dosing schedule. Paclitaxel was applied intravenously once per week upon its respective MTD (maximum tolerated dose), although the dose was reduced from day 19 after tumor inoculation to 75% of MTD, due to unexpected high response to paclitaxel. Animal body weight and tumor size were determined three times weekly. Treatment for all groups started at a tumor size of 29 mm², at day 6 after tumor cell inoculation. For combination treatment Compound A27 and paclitaxel were applied at the same day within a time frame of 4 hours. Animals of control and Compound A27 monotherapy groups were treated for a duration of 15 days. Animals of paclitaxel monotherapy and paclitaxel/Compound A27 combination treatment groups were treated for a duration of 33 days. At the end of the study after one final treatment plasma and tumors were sampled for PK analysis and final tumor weight was determined.

Compound A27 achieved no or weak monotherapy efficacy after 15 treatment days upon suboptimal (40% and 60% MTD) dosing with 2 mg/kg or 3 mg/kg twice daily for 2 days on/5 days off p.o., achieving T/C_{weight} of 0.98 or 0.88 and relative T/Cₐᵣₑₐ of 1.00 or 0.90, respectively (Table 8).

Both, paclitaxel monotherapy upon its MTD/75% MTD (20/15 mg/kg) applied for 1 day on/6 days off i.v. (intravenous(ly)) and paclitaxel combination therapy with Compound A27, dosed upon 40% and 60% of its MTD with 2 mg/kg and 3 mg/kg twice daily p.o. (per os, by mouth, oral(ly)) for 2 days on/5 days off, achieved comparable statistically significant reduction of tumor size compared to vehicle treated control group after 15 treatment days, showing relative T/Cₐᵣₑₐ between 0.04 and 0.07. Paclitaxel monotherapy and paclitaxel/Compound A27 combination groups were treated for another 18 days. After 33 treatment days statistically significant improvement of paclitaxel monotherapy efficacy was achieved in the paclitaxel/Compound A27 3 mg/kg twice daily p.o. 2 days on/5 days off combination treatment group. Although progressive disease was observed in both paclitaxel monotherapy and paclitaxel/Compound A27 combination treatment groups, a clear tumor growth delay upon combination treatment compared to paclitaxel monotherapy was observed in A2780cis ovarian tumors (Table 8).

In the 3 mg/kg twice daily intermittent mono-treated group transient body weight loss occurred. In the 3 mg/kg twice daily intermittent paclitaxel combination treatment group toxicity occurred on days 10 and 12 (death 2 of 10). Overall, tolerability of treatments was acceptable (Table 8).

In summary, this study demonstrates cooperativity of Mps-1 kinase inhibitor Compound A27 and paclitaxel in the paclitaxel intrinsically resistant ovarian carcinoma model A2780cis, achieving significant tumor growth delay compared to paclitaxel monotherapy.

TABLE 8
Mps-1 kinase inhibitor anti-tumor efficacy in combination with
paclitaxel in A2780cis xenografts in nude mice.
A2780cis human ovarian cancer xenograft model

| | | | | Max. | |
|---|---|---|---|---|---|
| | | | | Body | |
| | | | | weight | |
| | Dose and | T/C^{a} | T/C^{a} | loss^{b} | Fatal |
| Compound | Schedule | area | weight | (%) | Tox |
| Vehicle PEG400/ | 10 ml/kg | 1.00 | 1.00 | — | 0/10 |
| Ethanol/ | 2QD | | | | |
| Solutol 70:5:25 + | 2on/5off | | | | |
| Cremophor | p.o. + | | | | |
| 5%/Ethanol 5%/ | 10 ml/kg | | | | |
| Saline 90% | QD | | | | |
| | 1on/6off i.v. | | | | |
| Compound A27 | 2 mg/kg 2QD | 1.00 | 0.98 | −5.2 | 0/10 |
| | 2on/5off p.o. | | | | |
| Compound A27 | 3 mg/kg 2QD | 0.90 | 0.88 | −11.1 | 0/10 |
| | 2on/5off p.o. | | | | |
| Paclitaxel | 20/15 (d19) mg/kg | 0.06^{#} | — | −0.7 | 0/10 |
| | QD 2on/6off i.v. | | | | |
| Compound A27 + | 2 mg/kg 2QD | 0.04^{#} | — | −4.1 | 0/10 |
| Paclitaxel | 2on/5off | | | | |
| | p.o. + | | | | |
| | 20/15 (d19) mg/kg | | | | |
| | QD 1on/6off i.v. | | | | |
| Compound A27 + | 3 mg/kg 2QD | 0.07^{#,##} | — | −8.6 | 2/10 |
| Paclitaxel | 2on/5off | | | | |
| | p.o. + | | | | |
| | 20/15 (d19) mg/kg | | | | |
| | QD 1on/6off i.v. | | | | |

^{#}P < 0.05 (compared to vehicle group at day of vehicle group termination)
^{##}P < 0.05 (compared to Paclitaxel group at end of the study)
^{a}) T/C = Treatment/Control ratio, Calculated from relative mean tumor area at control dosing stop [(tumor area of treatment group at dosing stop)-(tumor area of treatment group at day before first treatment)] or mean final tumor weight.
^{b}) Body Weight Loss: the maximum mean body weight loss expressed as a percent of the starting weight of the animal. Weight loss greater than 20% is considered toxic.
QD = once daily
2QD = 2 times per day
PEG 400 = polyethylene glycol having an average molecular weight of 400
Cremophor = polyethoxylated castor oil

In Vivo Anti-Tumor Efficacy of Mps-1 Kinase Inhibitors of the Present Invention in Combination with Paclitaxel in NCI-H1299 Human NSCLC Model in Nude Mice

The effect of combination treatment of an Mps-1 kinase inhibitor of the present invention and paclitaxel was studied in the NCI-H1299 taxane intrinsically resistant human lung carcinoma (NSCLC) xenograft model in nude mice. Compound A27 was applied orally upon sub-optimal doses (40% and 60% of MTD) in combination in the twice daily intermittent (2 days on/5 days off) dosing schedule. Paclitaxel was applied intravenously once per week upon its respective MTD, although the dose was reduced from day 24 after tumor inoculation to 75% of MTD, due to unexpected high response to paclitaxel. Substances were formulated in optimal vehicles to achieve solutions. Animal body weight and tumor size were determined three times weekly. Treatment for all groups started at a tumor size of 30 mm², at day 8 after tumor cell inoculation. For combination treatment Compound A27 and paclitaxel were applied at the same day within a time frame of 4 hours. Animals of control and Compound A27 monotherapy groups were treated for a duration of 22 days. Animals of paclitaxel monotherapy and paclitaxel/Compound A27 combination treatment groups were treated for a duration of 36 days. At the end of the study after one final treatment plasma and tumors were sampled for PK analysis and final tumor weight was determined.

Compound A27 achieved no or weak monotherapy efficacy after 22 treatment days upon suboptimal (40% and 60% MTD) dosing with 2 mg/kg or 3 mg/kg twice daily for 2 days on/5 days off p.o., achieving T/C_{weight} of 1.18 or 0.74 and relative T/Cₐᵣₑₐ of 0.99 or 0.75, respectively (Table 9).

Both, paclitaxel monotherapy upon its MTD/75% MTD (20/15 mg/kg) applied for 1 day on/6 days off i.v. and paclitaxel combination therapy with Compound A27, dosed upon 40% and 60% of its MTD with 2 mg/kg and 3 mg/kg twice daily p.o. for 2 days on/5 days off, achieved statistically significant reduction of tumor size compared to vehicle treated control group after 22 treatment days, showing for paclitaxel alone relative T/Cₐᵣₑₐ 0.13 and for paclitaxel combination with Compound A27 upon 2 mg/kg or 3 mg/kg twice daily p.o. for 2 days on/5 days off relative T/Cₐᵣₑₐ 0.01 and −0.03, respectively. Paclitaxel monotherapy and paclitaxel/Compound A27 combination groups were treated for another 14 days. After 36 treatment days statistically significant improvement of paclitaxel monotherapy efficacy was achieved in the paclitaxel/Compound A27 3 mg/kg twice daily p.o. 2 days on/5 days off combination treatment group. Progressive disease was observed in paclitaxel monotherapy group, whereas paclitaxel/Compound A27 combination treatment groups showed clear signs of disease stabilization by induction of tumor growth stagnation, especially in the paclitaxel/Compound A27 3 mg/kg twice daily p.o. 2 days on/5 days group, in NCI-H1299 NSCLC tumors (Table 9).

In the 2 mg/kg and the 3 mg/kg twice daily intermittent paclitaxel combination treatment groups toxicity occurred on day 14 (death 2 of 10) and days 11, 14 and 15 (death 3 of 10), respectively. Overall, tolerability of treatments was acceptable (Table 9).

In summary, this study demonstrates cooperativity of Mps-1 kinase inhibitor Compound A27 and paclitaxel in the paclitaxel intrinsically resistant NSCLC model NCI-H1299, achieving significant improvement of paclitaxel monotherapy efficacy inducing disease stabilization.

TABLE 9
Mps-1 kinase inhibitor anti-tumor efficacy in combination with
paclitaxel in NCI-H1299 xenografts in nude mice.
NCI-H1299 human NSCLC xenograft model

| | | | | Max. Body | |
|---|---|---|---|---|---|
| | Dose and | T/C^{a} | T/C^{a} | weight loss^{b} | Fatal |
| Compound | Schedule | area | weight | (%) | Tox |
| Vehicle PEG400/ | 10 ml/kg | 1.00 | 1.00 | −0.6 | 0/10 |
| Ethanol/ | 2QD | | | | |
| Solutol 70:5:25 + | 2on/5off | | | | |
| Cremophor | p.o. + | | | | |
| 5%/Ethanol 5%/ | 10 ml/kg | | | | |
| Saline 90% | QD | | | | |
| | 1on/6off i.v. | | | | |
| Compound A27 | 2 mg/kg 2QD | 0.98 | 1.18 | −5.5 | 0/10 |
| | 2on/5off p.o. | | | | |
| Compound A27 | 3 mg/kg 2QD | 0.64 | 0.74 | −15.5 | 0/10 |
| | 2on/5off p.o. | | | | |
| Paclitaxel | 20/15 (d19) | 0.13^{#} | — | −1.9 | 0/10 |
| | mg/kg QD | | | | |
| | 2on/6off i.v. | | | | |
| Compound A27 + | 2 mg/kg 2QD | 0.01^{#} | — | −5.0 | 2/10 |
| Paclitaxel | 2on/5off | | | | |
| | p.o. + | | | | |
| | 20/15 (d19) | | | | |
| | mg/kg QD | | | | |
| | 1on/6off i.v. | | | | |
| Compound A27 + | 3 mg/kg 2QD | — | — | −9.9 | 3/10 |
| Paclitaxel | 2on/5off | 0.03^{#,##} | | | |
| | p.o. + | | | | |
| | 20/15 (d19) | | | | |
| | mg/kg QD | | | | |
| | 1on/6off i.v. | | | | |

^{#}P < 0.05 (compared to vehicle group at day of vehicle group termination)
^{##}P < 0.05 (compared to Paclitaxel group at end of the study)
^{a}) T/C = Treatment/Control ratio, Calculated from relative mean tumor area at control dosing stop [(tumor area of treatment group at dosing stop)-(tumor area of treatment group at day before first treatment)] or mean final tumor weight.
^{b}) Body Weight Loss: the maximum mean body weight loss expressed as a percent of the starting weight of the animal. Weight loss greater than 20% is considered toxic.

In Vivo Anti-Tumor Efficacy of an Mps-1 Kinase Inhibitor in Combination with Docetaxel in NCI-H1299 Human NSCLC Model in Nude Mice

The effect of combination treatment of an Mps-1 kinase inhibitor with docetaxel, another SAC activating, microtubule-destabilizing, anti-mitotic agent, used as standard of care in NSCLC patients, was studied in the NCI-H1299 taxane intrinsically resistant human lung carcinoma (NSCLC) xenograft model in nude mice.

The Mps-1 kinase inhibitor was applied orally upon sub-optimal doses (80% of MTD) in the optimized twice daily intermittent (2 days on/5 days off) dosing schedule in combination with docetaxel. Docetaxel was applied intravenously once per week upon its respective MTD. Substances were formulated in optimal vehicles to achieve solutions. Animal body weight and tumor size were determined two times weekly. Treatment for all groups started at a tumor size of 28 mm², at day 10 after tumor cell inoculation. For combination treatment the Mps-1 kinase inhibitor and docetaxel were applied at the same day within a time frame of 4 hours. Animals of control and Mps-inhibitor monotherapy group were treated for a duration of 20 days. Animals of docetaxel monotherapy and docetaxel/Mps-1 kinase inhibitor combination treatment groups were treated for a duration of 42 days. At the end of the study after one final treatment plasma and tumors were sampled for PK analysis and final tumor weight was determined.

In Vivo Anti-Tumor Efficacy of an Mps-1 Kinase Inhibitor in Combination with Paclitaxel in MDA-MB 231 Human Triple-Negative Breast Cancer Model in Nude Mice

The effect of combination treatment of an Mps-1 kinase inhibitor and paclitaxel was studied in the MDA-MB 231 human triple-negative (no expression of Her2/neu, progesterone receptor, estrogen receptor) xenograft model in nude mice.

The Mps-1 kinase inhibitor was applied orally upon sub-optimal doses (40% of MTD) in the optimized twice daily intermittent (2 days on/5 days off) dosing schedule in combination with paclitaxel. Paclitaxel was applied intravenously once per week upon its respective MTD. Substances were formulated in optimal vehicles to achieve solutions. Animal body weight and tumor size were determined three times weekly. Treatment for all groups started at a tumor size of 27 mm², at day 24 after tumor cell inoculation. For combination treatment, the Mps-1 kinase inhibitor and paclitaxel were applied at the same day within a time frame of 4 hours. Animals of control and monotherapy group (Mps-1 kinase inhibitor only) were treated for a duration of 28 days. Animals of paclitaxel monotherapy and paclitaxel/Mps-1 kinase inhibitor treatment groups were treated for a duration of 50 days. At the end of the study after one final treatment plasma and tumors were sampled for PK analysis and final tumor weight was determined.

Combination of an Mps-1 Kinase Inhibitor with Paclitaxel in the MKN1 Human Gastric Cancer Model

The effect of Compound A27 with paclitaxel combination treatment can be studied in the taxane-sensitive MKN1 human gastric carcinoma model in nude mice. Compound A27 can be administered p.o. in the 2QD intermittent (2 days on/5 days off) dosing schedule up to the respective MTD in single-agent treatment and at a dose of 40% of the single-agent MTD in combination.

Paclitaxel can be administered i.v. QW (once per week (meaning the 1 day on/6 days off treatment schedule)) at its respective MTD. Treatment for all groups can start on day 7 after tumor cell inoculation. For combination treatment, Compound A27 and paclitaxel can be administered on the same day within a time of 4 hours. Animals of the control and Compound A27 single-agent treatment groups can be treated for 40 days. Animals of the paclitaxel single-agent and Compound A27 with paclitaxel combination treatment groups can be treated for 78 days. Vehicle-treated control and Compound A27 single-agent treatment groups have to be terminated before reaching maximum tumor area due to MKN1 tumor-associated cachexia, inducing critical body weight loss and toxicity.

In summary, this study will demonstrate cooperativity of an Mps-1 kinase inhibitor and paclitaxel in the paclitaxel-sensitive gastric carcinoma model MKN1.

Combination of an Mps-1 Kinase Inhibitor with Vincristine in the MiaPaCa2 Human Pancreatic Tumor Model

Efficacy and tolerability of Mps-1 kinase inhibitor Compound A27 in combination with Paclitaxel can be evaluated in the MiaPaCa2 human pancreatic tumor model xenografted onto nude mice.

MiaPaCa2 cells obtained from cell culture can be implanted s.c. into the inguinal region of female nude mice. Treatment can be started when the tumors are 30-40 mm² in size. Tumor area can be determined by caliper measurements twice weekly. Treatment groups can be:
1) vehicle, PEG400/Ethanol/Solutol (70:5:25), bid 2on/5off p.o.
2) Compound A27, 0.45 mg/kg bid 2on/5off p.o.
3) Compound A27, 0.6 mg/kg bid 2on/5off p.o.
4) Paclitaxel, 24.0 mg/kg i.v. od 1on/6off
5) Compound A27, 0.45 mg/kg bid 2on/5off p.o.+Paclitaxel, 24.0 mg/kg i.v. od 1on/6off
6) Compound A27, 0.6 mg/kg bid 2on/5off p.o.+Paclitaxel, 24.0 mg/kg i.v. od 1on/6off

In summary, significant improvement of tumor growth inhibition by combination of Paclitaxel at MTD with low dose of Compound A27 at good tolerability compared to Paclitaxel monotherapy in the Taxane semi-sensitive pancreatic tumor model MiaPaCa2 will be demonstrated.

Combination of an Mps-1 Kinase Inhibitor with Vincristine in the Human Glioblastoma Model U87 MG

The dose dependent tumor-inhibiting effects of Compound A27 alone or in combination with vincristine can be investigated in the human glioblastoma model U87 MG, xenografted in nude mice.

The study is designed to determine the response of this glioblastoma model to the treatment with the investigational Compound A27 and vincristine, both alone at a fixed dose, and vincristine in combination with two different doses, both lower than that of Compound A27 used in the monotherapy schedule. The size of the glioblastoma can be used as read out parameter for response.

The cell culture derived human xenograft U87 MG can be initiated by transplantation of the tumor cells into the left hemisphere of the mouse brain. Treatment can be done in three cycles between day 3 and day 19. Mice can be sacrificed at day 24, the brain isolated and shock frozen in 2 methyl-butane. Tumor size can be determined as measure for tumor growth inhibition from cryo-slizes after staining.

In summary, the combination of Compound A27 with vincristine will result in a significant inhibition of tumor growth in the human U87-MG mouse xenograft, which will be better than the treatment with the single drugs. The treatment can be accompanied with severe gastrointestinal toxicity which is most likely caused by intolerance against the vehicle.

1. A combination comprising:
a compound A which is selected from:
N-cyclopropyl-4-{6-[1-(3-fluoro-4-methoxyphenyl)ethenyl]-8-[(3,3,3-trifluoropropyl)amino]imidazo[1,2-b]pyridazin-3-yl}-2-methylbenzamide,
N-cyclopropyl-4-{6-[difluoro(3-fluoro-4-methoxyphenyl)methyl]-8-[(3,3,3-trifluoropropyl)amino]imidazo[1,2-b]pyridazin-3-yl}-2-methylbenzamide,
(RS)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(R)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(S)—N-cyclopropyl-4-{6-[(3-fluoro-2-hydroxyphenyl)(hydroxy)methyl]-8-[(3,3,3-trifluoropropyl)amino]imidazo[1,2-b]pyridazin-3-yl}-2-methylbenzamide,
(RS)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(R)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(S)—N-cyclopropyl-4-{6-[1-(3-fluoro-2-hydroxyphenyl)-1-hydroxyethyl]-8-[(3,3,3-trifluoropropyl)amino]imidazo[1,2-b]pyridazin-3-yl}-2-methylbenzamide,
(RS)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
(R)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
(S)—N-cyclopropyl-4-{6-[fluoro(3-fluorophenyl)methyl]-8-[(3,3,3-trifluoropropyl)amino]imidazo[1,2-b]pyridazin-3-yl}-2-methylbenzamide,
N-cyclopropyl-4-{6-[difluoro(3-fluorophenyl)methyl]-8-[(3,3,3-trifluoropropyl)amino]imidazo[1,2-b]pyridazin-3-yl}-2-methylbenzamide,
N-cyclopropyl-4-{6-(3-methoxybenzoyl)-8-[(3, 3,3-trifluoropropyl)amino]imidazo[1,2-b]pyridazin-3-yl}-2-methylbenzamide,
N-cyclopropyl-4-{6-[1-(3-methoxyphenyl)vinyl]-8-[(3,3,3-trifluoropropyl)amino]imidazo[1,2-b]pyridazin-3-yl}-2-methylbenzamide,
N-cyclopropyl-4-{6-(4-methoxybenzoyl)-8-[(3,3,3-trifluoropropyl)amino]imidazo[1,2-b]pyridazin-3-yl}-2-methylbenzamide,
N-cyclopropyl-4-{6-[1-(4-methoxyphenyl)vinyl]-8-[(3, 3,3-trifluoropropyl)amino]imidazo[1, 2-b]pyridazin-3-yl}-2-methylbenzamide,
N-cyclopropyl-4-{6-[(2, 5-difluorophenyl)(hydroxy)methyl]-8-[(3,3,3-trifluoropropyl)amino]imidazo[1,2-b]pyridazin-3-yl}-2-methylbenzamide,
N-cyclopropyl-4-{6-(2, 5-difluorobenzoyl)-8-[(3,3,3-trifluoropropyl)amino]imidazo[1,2-b]pyridazin-3-yl}-2-methylbenzamide,
N-cyclopropyl-4-{6-(3-fluoro-4-methoxybenzoyl)-8-[(3,3,3-trifluoropropyl)amino]imidazo[1,2-b]pyridazin-3-yl}-2-methylbenzamide,
N-cyclopropyl-4-{6-(2,3-difluorobenzoyl)-8-[(3,3,3-trifluoropropyl)amino]imidazo[1,2-b]pyridazin-3-yl}-2-methylbenzamide,
N-cyclopropyl-4-{6-[1-(2,3-difluorophenyl)vinyl]-8-[(3,3,3-trifluoropropyl)amino]imidazo[1,2-b]pyridazin-3-yl}-2-methylbenzamide,
N-cyclopropyl-4-{6-[difluoro(4-methoxyphenyl)methyl]-8-[(3,3,3-trifluoropropyl)amino]imidazo[1,2-b]pyridazin-3-yl}-2-methylbenzamide,
N-cyclopropyl-4-{6-[1-(2,3-difluorophenyl)cyclopropyl]-8-[(3,3,3-trifluoropropyl)amino]imidazo[1,2-b]pyridazin-3-yl}-2-methylbenzamide,
N-cyclopropyl-4-{6-[(2,3-difluorophenyl)(difluoro)methyl]-8-[(3,3,3-trifluoropropyl)amino]imidazo[1,2-b]pyridazin-3-yl}-2-methylbenzamide,
N-cyclopropyl-4-{6-[1-(2, 5-difluorophenyl)vinyl]-8-[(3,3,3-trifluoropropyl)amino]imidazo[1,2-b]pyridazin-3-yl}-2-methylbenzamide,
N-cyclopropyl-4-{6-[1-(2, 5-difluorophenyl)cyclopropyl]-8-[(3,3,3-trifluoropropyl)amino]imidazo[1,2-b]pyridazin-3-yl}-2-methylbenzamide,
N-cyclopropyl-4-{6-[(2,5-difluorophenyl)(difluoro)methyl]-8-[(3,3,3-trifluoropropyl)amino]imidazo[1,2-b]pyridazin-3-yl}-2-methylbenzamide,
N-cyclopropyl-4-{6-[1-(5-fluoro-2-hydroxyphenyl)ethenyl]-8-[(3,3,3-trifluoropropyl)amino]imidazo[1,2-b]pyridazin-3-yl}-2-methylbenzamide,
N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide,
N-cyclopropyl-4-{6-(3-fluoro-4-methoxyphenoxy)-8-[(oxetan-3-ylmethyl)amino]imidazo[1,2-b]pyridazin-3-yl}-2-methylbenzamide,
N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide, and
N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide,
or an N-oxide, a hydrate, a solvate, or a salt thereof;
and
one or more mitotic inhibitors.

2. The combination according to claim 1, wherein the mitotic inhibitor is a vinca alkaloid.

3. The combination according to claim 1, wherein the mitotic inhibitor is a taxane.

4. The combination according to claim 1, wherein the mitotic inhibitor is selected from docetaxel and paclitaxel.

5. The combination according to claim 1, wherein the compound A is selected from:
N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide,
N-cyclopropyl-4-{6-(3-fluoro-4-methoxyphenoxy)-8-[(oxetan-3-ylmethyl)amino]imidazo[1,2-b]pyridazin-3-yl}-2-methylbenzamide,
N-cyclopropyl-4-{6-(2,3-difluoro-4-methoxyphenoxy)-8-[(3,3,3-trifluoropropyl)amino]imidazo[1, 2-b]pyridazin-3-yl}-2-methylbenzamide, and
N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide,
or an N-oxide, a hydrate, a solvate, or a salt thereof, or a mixture of same.

6. The combination according to claim 1, wherein the compound A is N-cyclopropyl-4-{6-[1-(5-fluoro-2-hydroxyphenyl)cyclopropyl]-8-[(3,3,3-trifluoropropyl)amino]imidazo[1,2-b]pyridazin-3-yl}-2-methylbenzamide, or an N-oxide, a hydrate, a solvate, or a salt thereof.

7. The combination according to claim 1, wherein the compound A is N-cyclopropyl-4-{6-(3-fluoro-4-methoxyphenoxy)-8-[(oxetan-3-ylmethyl)amino]imidazo[1,2-b]pyridazin-3-yl}-2-methylbenzamide,
or an N-oxide, a hydrate, a solvate, or a salt thereof.

8. The combination according to claim 1, wherein the compound A is N-cyclopropyl-4-{6-(2,3-difluoro-4-methoxyphenoxy)-8-[(3,3,3-trifluoropropyl)amino]imidazo[1,2-b]pyridazin-3-yl}-2-methylbenzamide,
or an N-oxide, a hydrate, a solvate, or a salt thereof.

9. The combination according to claim 1, wherein the compound A is N-cyclopropyl-4-{6-(2,3-difluoro-4-methoxyphenoxy)-8-[(tetrahydro-2H-pyran-4-ylmethyl)amino]imidazo[1,2-b]pyridazin-3-yl}-2-methylbenzamide, or an N-oxide, a hydrate, a solvate, or a salt thereof.

10. The combination according to claim 1, further comprising cisplatin.

11. (canceled)

12. (canceled)

13. A method of treatment of pancreatic cancer, glioblastoma, ovarian cancer, non-small cell lung carcinoma, breast cancer gastric cancer in a subject in need thereof, comprising administering to said subject a therapeutically effective amount of a combination according to claim 1.

14. A kit comprising a combination of:
component A: one or more compounds A, as defined in claim 1;
and
component B: one or more mitotic inhibitors;
and, optionally, one or more further pharmaceutical agents C; in which optionally all or either of said components A and B are in the form of a pharmaceutical formulation which is ready for use to be administered simultaneously, concurrently, separately or sequentially.

15. The kit according to claim 14, wherein the mitotic inhibitor is selected from docetaxel, paclitaxel and the optional pharmaceutical agent C is cisplatin.

16. The combination according to claim 2, wherein the vinca alkaloid is selected from vinblastine, vincristine, vindesine, vinorelbine, desoxyvincaminol, vincaminol, vinburnine, vincamajine, vineridine, and vinburnine.

17. The combination according to claim 3, wherein the taxane is selected from docetaxel, paclitaxel, and their analogues.

18. The kit according to claim 14, wherein the mitotic inhibitor is selected from docetaxel, paclitaxel, vinblastine, vincristine, vindesine, and vinorelbine.


SEQUENCE LISTING (1 sequence)

SEQ ID NO: 1 - protein, 14 aa, Artificial Sequence
  biotinylated peptide of the amino-acid sequence PWDPDDADITEILG
PWDPDDADITEILG